(19) United States
(12) Patent Application Publication
(10) Pub. No.: US 2025/0136643 A1
(43) Pub. Date: May 1, 2025

(54) HEPATITIS C VIRUS E1/E2 HETERODIMERS AND METHODS OF PRODUCING SAME

(71) Applicant: The Governors of the University of Alberta, Edmonton (CA)

(72) Inventors: Michael Houghton, Danville, CA (US); Darren Hockman, Edmonton (CA); John L. Law, Edmonton (CA); Chao Chen, Edmonton (CA); Michael Logan, Edmonton (CA)

(21) Appl. No.: 18/975,849

(22) Filed: Dec. 10, 2024

Related U.S. Application Data
(63) Continuation of application No. 17/509,808, filed on Oct. 25, 2021, now Pat. No. 12,202,859.
Application No. 17/509,808 is a continuation of application No. 15/763,370, filed on Mar. 26, 2018, now Pat. No. 11,186,615.
(60) Provisional application No. 62/239,157, filed on Oct. 8, 2015.
(60) Provisional application No. 62/337,212, filed on May 16, 2016.

(51) Int. Cl. C07K 14/005; A61K 39/00; A61K 39/12; A61K 39/29; A61P 31/14; C07K 1/22; C07K 19/00; C12N 7/00; C12N 15/62; C12P 21/02

(52) U.S. Cl. CPC C07K 14/005; A61K 39/12; A61K 39/29; A61P 31/14; C07K 1/22; C07K 19/00; C12N 7/00; C12N 15/62; C12P 21/02; A61K 2039/54; A61K 2039/55505; A61K 2039/55572; A61K 2039/575; C07K 2319/30; C07K 2319/50; C12N 2770/24222; C12N 2770/24234; C12N 2770/28122

(57) ABSTRACT

The present disclosure provides affinity tagged heterodimeric polypeptides comprising a hepatitis C virus (HCV) E1 polypeptide and an HCV E2 polypeptide, where one or both of the E1 and E2 polypeptides comprises an affinity tag. The present disclosure provides a method of producing an affinity tagged E1/E2 heterodimer of the present disclosure. The present disclosure provides methods of producing untagged HCV E1/E2 heterodimers. The present disclosure provides HCV E1/E2 heterodimers, compositions comprising same, and methods of inducing an immune response to HCV.

### CROSS-REFERENCE

This application is a continuation of U.S. application Ser. No. 17/509,808, filed Oct. 25, 2021, which is a continuation of U.S. application Ser. No. 15/763,370, filed Mar. 26, 2018, issued as U.S. Pat. No. 11,186,615, which application is a 371 of international Application No. PCT/IB2016/056000, filed Oct. 7, 2016, which claims the benefit of U.S. Provisional Patent Application No. 62/239,157, filed Oct. 8, 2015, and U.S. Provisional Patent Application No. 62/337,212, filed May 16, 2016, which applications are incorporated herein by reference in their entirety.

### INCORPORATION BY REFERENCE OF SEQUENCE LISTING PROVIDED AS A SEQUENCE LISTING XML FILE

A Sequence Listing is provided herewith as a Sequence Listing XML, “UALB-030CON2_SEQ_LIST.xml”, created on Dec. 9, 2024 and having a size of 170,914 bytes. The contents of the Sequence Listing XML are incorporated herein by reference in their entirety.

### INTRODUCTION

Hepatitis C virus (HCV) is a blood-borne pathogen that is estimated to infect 150-200 million people worldwide. Infection by HCV may be non-symptomatic, and can be cleared by patients, sometimes without medical intervention. However, the majority of patients develop a chronic HCV infection, which may lead to liver inflammation, scarring, and even to liver failure or liver cancer. In the United States alone, over 3 million people have a chronic infection.

The HCV virion contains a positive-sense single stranded RNA genome of about 9.5 kb. The genome encodes a single polyprotein of 3,010 to 3,030 amino acids. The structural proteins comprise a core protein forming the viral nucleocapsid and two envelope glycoproteins, E1 and E2.

There is a need in the art for compositions and methods for inducing immune responses to HCV.

### SUMMARY

The present disclosure provides a method to facilitate large-scale purification of the hepatitis C virus (HCV) E1E2 glycoprotein heterodimer using affinity purification tags, for example the human fragment crystallizable region (Fc region) of immunoglobulins (human or from other species) fused to the N-terminus of E2. Following affinity purification of the E1E2 heterodimer complex (for example, using Protein A or Protein G or Protein L affinity columns), the Fc domain can be removed from the E1E2 complex by digestion with a suitable protease at a proteolytic cleavage site placed downstream of the Fc domain. Ordinarily, the large scale purification of E1E2 is challenging which limits its use as a HCV vaccine in humans. The present disclosure addresses this challenge. The present disclosure also provides various affinity purification-tagged heterodimeric polypeptides comprising an HCV E1 polypeptide and an HCV E2 polypeptide, where one or both of the E1 and E2 polypeptides comprises an affinity purification tag. The present disclosure provides a method of producing an affinity tagged E1/E2 heterodimer of the present disclosure. The present disclosure provides methods of producing untagged HCV E1/E2 heterodimers. The present disclosure also provides HCV E1/E2 heterodimers, compositions comprising the HCV E1/E2 heterodimers, and methods of inducing an immune response to HCV; where the HCV E1/E2 heterodimers comprise a variant HCV E1 polypeptide or a variant HCV E2 polypeptide, the variant E1 or E2 polypeptide comprising from 1 to 6 amino acids from a proteolytically cleavable linker.

The present disclosure provides an affinity tagged heterodimeric polypeptide comprising: a) a hepatitis C virus (HCV) E1 polypeptide; and b) an HCV E2 polypeptide, wherein at least one of the HCV E1 and HCV E2 polypeptides is a fusion polypeptide comprising an affinity tag polypeptide. In some cases, the affinity tagged heterodimeric polypeptide comprises a proteolytically cleavable linker interposed between the affinity tag polypeptide and the HCV E1 or HCV E2 polypeptide. In some cases, a) the proteolytically cleavable linker comprises the sequence LEVLFQGP (SEQ ID NO:1), wherein cleavage occurs between the glutamine and the glycine; b) the proteolytically cleavable linker comprises the sequence ENLYFQS (SEQ ID NO: 2), wherein cleavage occurs between the glutamine and the serine; c) the proteolytically cleavable linker comprises the sequence DDDDK (SEQ ID NO:3), wherein cleavage occurs immediately C-terminal to the lysine residue; or d) the proteolytically cleavable linker comprises the sequence LVPR (SEQ ID NO:4) (e.g., where the proteolytically cleavable linker comprises the sequence LVPRGS (SEQ ID NO:5)). In some cases, the affinity tag is an immunoglobulin (Ig) Fc polypeptide, Protein A, Protein G, a hybrid Protein A-Protein G polypeptide, a Protein L polypeptide, a polypeptide comprising a poly(histidine) tract, an immunoglobulin light chain, or glutathione-S-transferase. In some cases, the affinity tagged heterodimeric polypeptide comprises: a) an HCV E1-affinity tag fusion polypeptide comprising an HCV E1 polypeptide and an affinity tag polypeptide; and b) an HCV E2 polypeptide. In some cases, the affinity tagged heterodimeric polypeptide comprises: a) an HCV E1-Fc fusion polypeptide comprising an HCV E1 polypeptide and an Ig Fc polypeptide; and b) an HCV E2 polypeptide. In some cases, the HCV E1-Fc fusion polypeptide comprises, in order from N-terminus to C-terminus: i) the HCV E1 polypeptide; and ii) the Ig Fc polypeptide. In some cases, the HCV E1-Fc fusion polypeptide comprises, in order from N-terminus to C-terminus: i) the Ig Fc polypeptide; and ii) the HCV E1 polypeptide. In some cases, the affinity tagged heterodimeric polypeptide comprises: a) an HCV E2-affinity tag fusion polypeptide comprising an HCV E2 polypeptide and an affinity tag polypeptide; and b) an HCV E1 polypeptide. In some cases, the affinity tagged heterodimeric polypeptide comprises: a) an HCV E2-Fc fusion polypeptide comprising an HCV E2 polypeptide and an Ig Fc polypeptide; and b) an HCV E1 polypeptide. In some cases, the HCV E2-Fc fusion polypeptide comprises, in order from N-terminus to C-terminus: i) the HCV E2 polypeptide; and ii) the Ig Fc polypeptide. In some cases, the HCV E2-Fc fusion polypeptide comprises, in order from N-terminus to C-terminus: i) the Ig Fc polypeptide; and ii) the HCV E2 polypeptide. In some cases, the affinity tagged heterodimeric polypeptide comprises: a) an HCV E1-affinity tag fusion polypeptide comprising an HCV E1 polypeptide and an affinity tag polypeptide; and b) an HCV E2-affinity tag fusion polypeptide comprising an HCV E2 polypeptide and an affinity tag polypeptide. In some cases, the affinity tagged heterodimeric polypeptide comprises: a) an HCV E1-Fc fusion polypeptide comprising an HCV E1 polypeptide and an Ig Fc polypeptide; and b) an HCV E2-Fc fusion polypeptide comprising an HCV E2 polypeptide and an Ig Fc polypeptide. In some cases, a) the HCV E1-Fc fusion polypeptide comprises, in order from N-terminus to C-terminus: i) the HCV E1 polypeptide; and ii) the Ig Fc polypeptide; and b) the HCV E2-Fc fusion polypeptide comprises, in order from N-terminus to C-terminus: i) the HCV E2 polypeptide; and ii) the Ig Fc polypeptide. In some cases: a) the HCV E1-Fc fusion polypeptide comprises, in order from N-terminus to C-terminus: i) the Ig Fc polypeptide; and ii) the HCV E1 polypeptide; and b) the HCV E2-Fc fusion polypeptide comprises, in order from N-terminus to C-terminus: i) the Ig Fc polypeptide; and ii) the HCV E2 polypeptide. In some cases, the HCV E2 polypeptide comprises an amino acid sequence having at least 20% amino acid sequence identity to an E2 polypeptide depicted in one of FIG. 1A-1C, FIGS. 2A-2C, FIGS. 3A-3C, and FIGS. 4A-4B. In some cases, the HCV E1 polypeptide comprises an amino acid sequence having at least 20% amino acid sequence identity to an E1 polypeptide depicted in FIGS. 1A-1C, FIGS. 2A-2C, FIGS. 3A-3C, and FIGS. 4A-4B. In some cases, the Ig Fc polypeptide comprises an amino acid sequence having at least 85% amino acid sequence identity to an amino acid sequence depicted in FIGS. 5A-5C. In some cases, an affinity tagged heterodimeric polypeptide of comprising an affinity tagged HCV E2 polypeptide, wherein the HCV E2-affinity tag fusion polypeptide comprises, in order from N-terminus to C-terminus: i) from 2 to 15 amino acids of the N-terminus of an HCV E2 polypeptide; ii) the affinity tag polypeptide, iii) a proteolytically cleavable linker; and iv) an HCV E2 polypeptide. In some cases, the from 2 to 15 amino acids of the N-terminus of an HCV E2 polypeptide is a dipeptide selected from QT, ET, HT, GT, TT, RH, NT, AY, VI, and ST. In some cases: a) the proteolytically cleavable linker comprises the sequence LEVLFQGP (SEQ ID NO:1), wherein cleavage occurs between the glutamine and the glycine; b) the proteolytically cleavable linker comprises the sequence ENLYFQS (SEQ ID NO:2), wherein cleavage occurs between the glutamine and the serine; c) the proteolytically cleavable linker comprises the sequence DDDDK (SEQ ID NO:3), wherein cleavage occurs immediately C-terminal to the lysine residue; or d) the proteolytically cleavable linker comprises the sequence LVPR (SEQ ID NO:4) (e.g., where the proteolytically cleavable linker comprises the sequence LVPRGS (SEQ ID NO:5)). In some cases, the HCV E2 polypeptide and the HCV E1 polypeptide are of the same genotype. In some cases, the HCV E2 polypeptide and the HCV E1 polypeptide are of different genotypes. In some cases, the HCV E2 polypeptide is of genotype 1, 2, 3, or 7. In some cases, the HCV E1 polypeptide is of genotype 1, 2, 3, or 7.

The present disclosure provides a composition comprising: a) a heterodimeric polypeptide as described above or elsewhere herein; and b) a buffer. The composition can include a protease inhibitor.

The present disclosure provides a nucleic acid comprising: a) a first nucleotide sequence encoding a hepatitis C virus (HCV) E1 polypeptide; and b) a second nucleotide sequence encoding an HCV E2 polypeptide, wherein at least one of the HCV E1 and HCV E2 polypeptides is a fusion polypeptide comprising an affinity tag polypeptide. In some cases, the nucleic acid comprises a nucleotide sequence encoding a polyprotein comprising, in order from N-terminus to C-terminus: a) an HCV E1 polypeptide; and b) an HCV E2-affinity tag fusion polypeptide comprising, in order from N-terminus to C-terminus: i) an affinity tag polypeptide; ii) a proteolytically cleavable linker; and iii) an HCV E2 polypeptide. In some cases, a nucleotide sequence encoding from 2 amino acids to 15 amino acids from the N-terminus of an HCV E2 polypeptide is interposed between the HCV E1 polypeptide and the affinity tag polypeptide such that a wild-type signal protease E1/E2 junction cleavage site is generated between the HCV E1 polypeptide and the affinity tag polypeptide. In some cases, the nucleic acid comprises a nucleotide sequence encoding a polyprotein comprising, in order from N-terminus to C-terminus: a) an HCV E1 polypeptide; and b) an HCV E2-affinity tag fusion polypeptide comprising, in order from N-terminus to C-terminus: i) an HCV E2 polypeptide; ii) a proteolytically cleavable linker; and iii) an affinity tag polypeptide. In some cases, the nucleic acid comprises a nucleotide sequence encoding a polyprotein comprising, in order from N-terminus to C-terminus: a) an HCV E1-affinity tag fusion polypeptide comprising, in order from N-terminus to C-terminus: i) an affinity tag polypeptide; ii) a proteolytically cleavable linker; and iii) an HCV E1 polypeptide; and b) an HCV E2 polypeptide. In some cases, the nucleic acid comprises a nucleotide sequence encoding a polyprotein comprising, in order from N-terminus to C-terminus: a) an HCV E1-affinity tag fusion polypeptide comprising, in order from N-terminus to C-terminus: i) an HCV E1 polypeptide; ii) a proteolytically cleavable linker; and iii) an affinity tag polypeptide; and b) an HCV E2 polypeptide. In some cases, a nucleotide sequence encoding a signal peptide is interposed between the affinity tag polypeptide and the HCV E2 polypeptide such that signal protease cleavage site is generated between the affinity tag polypeptide and the HCV E2 polypeptide. In some cases, the nucleic acid comprises a nucleotide sequence encoding a polyprotein comprising, in order from N-terminus to C-terminus: a) an HCV E1-affinity tag fusion polypeptide comprising, in order from N-terminus to C-terminus: i) an affinity tag polypeptide; ii) a proteolytically cleavable linker; and iii) an HCV E1 polypeptide; and b) an HCV E2-affinity tag fusion polypeptide comprising, in order from N-terminus to C-terminus: i) an affinity tag polypeptide; ii) a proteolytically cleavable linker; and iii) an HCV E2 polypeptide. In some cases, a nucleotide sequence encoding from 2 amino acids to 15 amino acids from the N-terminus of an HCV E2 polypeptide is interposed between the HCV E1 polypeptide and the affinity tag polypeptide such that a wild-type signal protease E1/E2 junction cleavage site is generated between the HCV E1 polypeptide and the affinity tag polypeptide. In some cases, the nucleic acid comprises a nucleotide sequence encoding a polyprotein comprising, in order from N-terminus to C-terminus: a) an HCV E1-affinity tag fusion polypeptide comprising, in order from N-terminus to C-terminus: i) an HCV E1 polypeptide; ii) a proteolytically cleavable linker; and iii) an affinity tag polypeptide; and b) an HCV E2-affinity tag fusion polypeptide comprising, in order from N-terminus to C-terminus: i) an HCV E2 polypeptide; ii) a proteolytically cleavable linker; and iii) an affinity tag polypeptide. In some cases, a nucleotide sequence encoding a signal peptide is interposed between the affinity tag polypeptide and the HCV E2 polypeptide such that a signal protease cleavage site is generated between the affinity tag polypeptide and the HCV E2 polypeptide. In some cases, the nucleotide sequence encoding the polyprotein is operably linked to a promoter. In some cases, the affinity tag polypeptide is an Ig Fc polypeptide. In some cases, the Ig Fc polypeptide comprises an amino acid sequence having at least 85% amino acid sequence identity to an amino acid sequence depicted in FIG. 5A-5C. In some cases, the from 2 to 15 amino acids of the N-terminus of an HCV E2 polypeptide is a dipeptide selected from QT, ET, HT, GT, TT, RH, NT, AY, VI, and ST. In some cases, a) the proteolytically cleavable linker comprises the sequence LEVLFQGP (SEQ ID NO:1), wherein cleavage occurs between the glutamine and the glycine; b) the proteolytically cleavable linker comprises the sequence ENLYFQS (SEQ ID NO:2, wherein cleavage occurs between the glutamine and the serine; c) the proteolytically cleavable linker comprises the sequence DDDDK (SEQ ID NO:3), wherein cleavage occurs immediately C-terminal to the lysine residue; or d) the proteolytically cleavable linker comprises the sequence LVPR (SEQ ID NO:4) (e.g., where the proteolytically cleavable linker comprises the sequence LVPRGS (SEQ ID NO:5)). In some cases, the HCV E2 polypeptide comprises an amino acid sequence having at least 20% amino acid sequence identity to an E2 polypeptide depicted in one of FIG. 1A-1C, FIG. 2A-2C, FIG. 3A-3C, and FIG. 4A-4B. In some cases, the HCV E1 polypeptide comprises an amino acid sequence having at least 20% amino acid sequence identity to an E1 polypeptide depicted in FIG. 1A-1C, FIG. 2A-2C, FIG. 3A-3C, and FIG. 4A-4B.

The present disclosure provides a recombinant expression vector comprising a nucleic acid as described above, or elsewhere herein. The present disclosure provides a genetically modified in vitro host cell comprising a nucleic acid as described above, or elsewhere herein. The present disclosure provides a genetically modified in vitro host cell comprising a recombinant expression vector as described above, or elsewhere herein. In some cases, the host cell is a eukaryotic cell. In some cases, the host cell is a mammalian cell.

The present disclosure provides a method of making an affinity tagged heterodimeric polypeptide as described above, or elsewhere herein, the method comprising: a) culturing a genetically modified host cell as described above, or elsewhere herein, under conditions such that the affinity tagged HCV E1-E2 heterodimer is produced in the cell; and b) lysing the cell to generate a cell lysate comprising the affinity tagged HCV E1-E2 heterodimer.

The present disclosure provides a method of producing an HCV E1/E2 heterodimer, the method comprising: a) contacting a lysate of the genetically modified host cell as described above, or elsewhere herein, with an affinity tag-binding polypeptide immobilized on an insoluble support, wherein the affinity tagged HCV E1-E2 heterodimer present in the lysate binds to the immobilized affinity tag-binding polypeptide, generating an immobilized affinity tagged HCV E1-E2 heterodimer; b) contacting the immobilized HCV E1-E2 heterodimer with a protease that cleaves the proteolytically cleavable linker present in the immobilized affinity tagged HCV E1-E2 heterodimer, thereby releasing the HCV E1-E2 heterodimer from the affinity tag; and c) collecting the released HCV E1-E2 heterodimer. In some cases, the released HCV E1-E2 heterodimer is at least 50% pure. In some cases, the affinity tag polypeptide is an Ig Fc polypeptide, and wherein the affinity tag-binding polypeptide is an Fc-binding polypeptide. In some cases, the Fc-binding polypeptide is Protein A, Protein G, or a Protein A/G fusion.

The present disclosure provides an affinity tagged heterodimeric polypeptide comprising: a) a hepatitis C virus (HCV) E1 polypeptide; and b) an affinity-tagged HCV E2 polypeptide, wherein the affinity-tagged HCV E2 polypeptide comprises, in order from N-terminus to C-terminus: i) an Ig Fc polypeptide; ii) a proteolytically cleavable linker having the amino acid sequence LEVLFQGP (SEQ ID NO:1); and iii) an HCV E2 polypeptide. In some cases, the HCV E1 polypeptide is an AVI1a129 E1 polypeptide, an H77 E1 polypeptide, an S52 E1 polypeptide, or an AVI3a177 E1 polypeptide, where the AVI1a129 E1 polypeptide, the H77 E1 polypeptide, the S52 E1 polypeptide, and the AVI3a177 E1 polypeptide comprise amino acid sequences as depicted in FIG. 7 and FIG. 8. In some cases, the HCV E2 polypeptide is an AVI1a129 E2 polypeptide, an H77 E1 polypeptide, an S52 E2 polypeptide, or an AVI3a177 E2 polypeptide, where the AVI1a129 E2 polypeptide, the H77 E2 polypeptide, the S52 E2 polypeptide, and the AVI3a177 E2 polypeptide comprise amino acid sequences as depicted in FIG. 7 and FIG. 8.

The present disclosure provides a nucleic acid comprising a nucleotide sequence encoding an affinity tagged heterodimeric polypeptide, wherein the nucleotide sequence comprises, from 5′ to 3′ and in operable linkage: a) a nucleotide sequence encoding a tissue plasminogen signal sequence having the amino acid sequence MDAMKRGLCCVLLLCGAVFVSPS (SEQ ID NO: 6); b) a nucleotide sequence encoding a hepatitis C virus (HCV) E1 polypeptide; c) a nucleotide sequence encoding a dipeptide selected from QT and ET; and d) a nucleotide sequence encoding an affinity-tagged HCV E2 polypeptide, wherein the affinity-tagged HCV E2 polypeptide comprises, in order from N-terminus to C-terminus: i) an Ig Fc polypeptide; ii) a proteolytically cleavable linker having the amino acid sequence LEVLFQGP (SEQ ID NO:1); and iii) an HCV E2 polypeptide. In some cases, the HCV E1 polypeptide is an AVI1a129 E1 polypeptide, an H77 E1 polypeptide, an S52 E1 polypeptide, or an AVI3a177 E1 polypeptide, where the AVI1a129 E1 polypeptide, the H77 E1 polypeptide, the S52 E1 polypeptide, and the AVI3a177 E1 polypeptide comprise amino acid sequences as depicted in FIG. 7 and FIG. 8. In some cases, the HCV E2 polypeptide is an AVI1a129 E2 polypeptide, an H77 E1 polypeptide, an S52 E2 polypeptide, or an AVI3a177 E2 polypeptide, where the AVI1a129 E2 polypeptide, the H77 E2 polypeptide, the S52 E2 polypeptide, and the AVI3a177 E2 polypeptide comprise amino acid sequences as depicted in FIG. 7 and FIG. 8. In some cases, the nucleic acid comprises a nucleotide sequence encoding an amino acid sequence depicted in FIG. 7 or FIG. 8. The present disclosure provides a recombinant expression vector comprising the nucleic acid. The present disclosure provides a mammalian host cell genetically modified with a recombinant expression vector comprising the nucleic acid. The present disclosure provides a method of producing an HCV E1/E2 heterodimer, the method comprising: a) contacting a lysate of the genetically modified host cell with a Protein A or Protein G polypeptide immobilized on an insoluble support, wherein the affinity tagged HCV E1-E2 heterodimer present in the lysate binds to the immobilized Protein A or Protein G, generating an immobilized affinity tagged HCV E1-E2 heterodimer; b) contacting the immobilized HCV E1-E2 heterodimer with a protease that cleaves the proteolytically cleavable linker present in the immobilized affinity tagged HCV E1-E2 heterodimer, thereby releasing the HCV E1-E2 heterodimer from the affinity tag; and c) collecting the released HCV E1-E2 heterodimer. In some cases, the released HCV E1-E2 heterodimer is at least 50% pure. In some cases, the released HCV E1-E2 heterodimer is at least 75% pure. In some cases, the released HCV E1-E2 heterodimer is at least 90% pure. In some cases, the released HCV E1-E2 heterodimer is at least 95% pure.

The present disclosure provides a heterodimeric polypeptide comprising: a) an HCV E1 polypeptide; and b) a modified E2 polypeptide comprising, in order from N-terminus to C-terminus: i) from 1 to 6 heterologous amino acids, wherein the from 1 to 6 heterologous amino acids are C-terminal to a site of proteolytic cleavage in a proteolytically cleavable linker; and ii) an HCV E2 polypeptide. The present disclosure provides a heterodimeric polypeptide comprising: a) an HCV E2 polypeptide; and b) a modified E1 polypeptide comprising, in order from N-terminus to C-terminus: i) from 1 to 6 heterologous amino acids, wherein the from 1 to 6 heterologous amino acids are C-terminal to a site of proteolytic cleavage in a proteolytically cleavable linker; and ii) an HCV E1 polypeptide. In some cases, the from 1 to 6 heterologous amino acids are Gly-Pro. In some cases, the from 1 to 6 heterologous amino acids is Ser or Gly. In some cases, the from 1 to 6 heterologous amino acids are Gly-Ser. The present disclosure provides a composition comprising: a) the heterodimeric polypeptide; and b) a pharmaceutically acceptable excipient. In some cases, the pharmaceutically acceptable excipient comprises an adjuvant. In some cases, the adjuvant is MF59; alum; poly(DL-lactide co-glycolide); a CpG oligonucleotide; keyhole limpet hemocyanin; or a suspension of liposomes comprising 3′-O-desacyl-4′-monophosphoryl lipid A (MPL) and Quillaja saponaria 21 (QS21).

The present disclosure provides a heterodimeric polypeptide comprising: a) an HCV E1 polypeptide; and b) a modified E2 polypeptide comprising, in order from N-terminus to C-terminus: i) an HCV E2 polypeptide; and ii) from 1 to 6 heterologous amino acids, wherein the from 1 to 6 heterologous amino acids are N-terminal to a site of proteolytic cleavage in a proteolytically cleavable linker. The present disclosure provides a heterodimeric polypeptide comprising: a) an HCV E2 polypeptide; and b) a modified E1 polypeptide comprising, in order from N-terminus to C-terminus: i) an HCV E1 polypeptide; and ii) from 1 to 6 heterologous amino acids, wherein the from 1 to 6 heterologous amino acids are N-terminal to a site of proteolytic cleavage in a proteolytically cleavable linker. In some cases, the from 1 to 6 heterologous amino acids are DDDDK (SEQ ID NO:3). In some cases, the from 1 to 6 heterologous amino acids are LEVLFQ (SEQ ID NO:7). In some cases, the from 1 to 6 heterologous amino acids are ENLYFQ (SEQ ID NO:8). In some cases, the from 1 to 6 heterologous amino acids are LVPR (SEQ ID NO:4). The present disclosure provides a composition comprising: a) the heterodimeric polypeptide; and b) a pharmaceutically acceptable excipient. In some cases, the pharmaceutically acceptable excipient comprises an adjuvant. In some cases, the adjuvant is MF59; alum; poly(DL-lactide co-glycolide); a CpG oligonucleotide; keyhole limpet hemocyanin; or a suspension of liposomes comprising 3′-O-desacyl-4′-monophosphoryl lipid A (MPL) and Quillaja saponaria 21 (QS21).

The present disclosure provides a method of inducing an immune response in an individual to an HCV polypeptide, the method comprising administering to the individual an effective amount of a heterodimeric polypeptide as described above or elsewhere herein, or a composition comprising a heterodimeric polypeptide, as described above or elsewhere herein. In some cases, said administering is by intramuscular administration. In some cases, said administering is by subcutaneous administration.

### BRIEF DESCRIPTION OF THE DRAWINGS

FIG. 1A-1C provide an amino acid sequence alignment of examples of the core-E1-E2 coding regions of a HCV genotype 1 virus, specifically representative HCV 1A, 1B and 1C genotypes. Genbank database sequences for the coding region core-E1-E2 were aligned using Geneious software v5.6.4. Numbering of amino acids is according to strain NP_671941 (H77). Consensus: SEQ ID NO:46; AVI1a129: SEQ ID NO:47; NP_671491 (H77): SEQ ID NO:48; EU155269: SEQ ID NO:49; EU781810: SEQ ID NO:50; EU781771: SEQ ID NO:51; AB520610: SEQ ID NO: 52; EU781752: SEQ ID NO:53; EU781759: SEQ ID NO:54; EF407439: SEQ ID NO:55; EF407427: SEQ ID NO:56; EU362905: SEQ ID NO:57; EF407413: SEQ ID NO:58; EU781808: SEQ ID NO:59; EU781790: SEQ ID NO:60; AJ238799 (Con1): SEQ ID NO:61; AAK97744: SEQ ID NO:62; AF139594: SEQ ID NO:63; AF176573: SEQ ID NO:64; BAA19625: SEQ ID NO: 65; BAA25076: SEQ ID NO:66; BAC54896: SEQ ID NO:67; BAD91386: SEQ ID NO:68; BAF46764: SEQ ID NO:69; BAG30950: SEQ ID NO: 70; CAB41951: SEQ ID NO:71; AAK95832: SEQ ID NO: 72; AAT69968: SEQ ID NO: 73; and BAA03581: SEQ ID NO:74.

FIG. 2A-2C provide an alignment of amino acid sequences of the core-E1-E2 coding region of representative HCV 2A and HCV2B subtypes. Genbank database sequences for the coding region core-E1-E2 were aligned using Geneious software v5.6.4. The amino acid numbering depicted is in accordance to the common HCV strains: ABO47639 (JFH1) and HPCJ8G-J8 (J8) for HCV2A and HCV2B, respectively. Consensus: SEQ ID NO:75; AB047639 (JFH1): SEQ ID NO: 76; AB047645: SEQ ID NO:77; AF169005: SEQ ID NO:79; AF238482: SEQ ID NO:78; AY746460: SEQ ID NO:80; HPCPOLP: SEQ ID NO:81; NC_009823: SEQ ID NO:82; HPCJ8G HC-J8: SEQ ID NO: 83; AB030907: SEQ ID NO: 86; AY232730: SEQ ID NO:84; AY232747: SEQ ID NO:85; and DQ430817: SEQ ID NO:87.

FIG. 3A-3C provide an amino acid sequence alignment of the core-E1-E2 coding region for representative HCV 3A, 3B and 3K genotypes. Genbank database sequences for the coding region core-E1-E2 were aligned using Geneious software v5.6.4. Consensus: SEQ ID NO:88; AVI3a177: SEQ ID NO:89; ADF97232 (S52): SEQ ID NO:90; YP_0014696: SEQ ID NO:91; CAA54244: SEQ ID NO:92; AAC03058: SEQ ID NO:93; AAY29642: SEQ ID NO:94; ABD85062: SEQ ID NO:95; ABD85063: SEQ ID NO:96; ABD97104: SEQ ID NO:97; BAA06044: SEQ ID NO:98; BAA08372: SEQ ID NO:99; and BAA09890: SEQ ID NO: 100.

FIG. 4A-4B provide an amino acid sequence of the core-E1-E2 coding region for HCV genotype 7a. Amino acid sequence for the coding region core-E1-E2 of genotype 7a (isolate QC69; Genbank: ABN05226.1; SEQ ID NO:101) is shown according to the numbering scheme of the reference strain, NP_671941 (H77).

FIG. 5A-5C provide amino acid sequences of immunoglobulin Fc regions. 3S7G_A: SEQ ID NO: 102; AAN76044: SEQ ID NO: 103; AAW65947: SEQ ID NO:104; AAA52770: SEQ ID NO: 105; 0308221A: SEQ ID NO:106; P01876: SEQ ID NO:107; 1F6A_B: SEQ ID NO:108 and P01861: SEQ ID NO:109.

FIG. 6A-6B provide a schematic representation of Fc tagged E2 polypeptides in a full-length E1-E2 construct (FIG. 6A); and gel depicting purification of untagged HCV E1/E2 heterodimer from CHO extracts expressing Fc-tagged E1/E2 heterodimer. Sequences from top to bottom: SEQ ID NOs: 110-116.

FIG. 7 provides an amino acid sequence alignment of Fc-tagged E1-E2 polyprotein of H77 (SEQ ID NO: 118) and Alberta isolate Avi1a129 (SEQ ID NO:117).

FIG. 8 provides an amino acid sequence alignment of Fc-tagged E1-E2 polyprotein of S52 (SEQ ID NO: 119) and Alberta isolate Avi3a177 (SEQ ID NO:120).

FIG. 9 depicts binding of neutralizing anti-HCV E2 monoclonal antibodies to recombinant E1/E2 antigens produced according to a method of the present disclosure.

FIG. 10A-10C provide amino acid sequences of Protein A (SEQ ID NO:121), Protein G (SEQ ID NO: 122), and Protein L (SEQ ID NO:123) polypeptides.

FIG. 11A-11D depict purification of E1E2 heterodimer from an Fc-tagged precursor. FIG. 11A depicts the proteolytically cleavable linker LEVLFQGP (SEQ ID NO:1).

FIG. 12A-12B depict induction of neutralizing antibodies by vaccination with wild type (WT) E1/E2 heterodimer and with Fc-tag-derived (Fc-d) HCV E1/E2 heterodimer.

FIG. 13 depicts a comparison of neutralizing antibodies toward homologous (genotype 1a) and heterologous (genotype 5a) HCV polyprotein (HCVpp).

FIG. 14 depicts competition by mouse antisera with HCV cross-neutralizing monoclonal antibodies (mAb) for binding to wild-type (WT) or Fc-tag-derived (Fc-d) HCV E1/E2 heterodimer.

### DEFINITIONS

The term “hepatitis C virus” (“HCV”), as used herein, refers to any one of a number of different genotypes and isolates of hepatitis C virus. Thus, “HCV” encompasses any of a number of genotypes, subtypes, or quasispecies, of HCV, including, e.g., genotype 1, 2, 3, 4, 6, 7, etc. and subtypes (e.g., 1a, 1b, 2a, 2b, 3a, 4a, 4c, etc.), and quasispecies. Representative HCV genotypes and isolates include: the “Chiron” isolate HCV-1, H77, J6, Con1, isolate 1, BK, EC1, EC10, HC-J2, HC-J5; HC-J6, HC-J7, HC-J8, HC-JT, HCT18, HCT27, HCV-476, HCV-KF, “Hunan”, “Japanese”, “Taiwan”, TH, type 1, type 1a, H77 type 1b, type 1c, type 1d, type 1e, type 1f, type 10, type 2, type 2a, type 2b, type 2c, type 2d, type 2f, type 3, type 3a, type 3b, type 3g, type 4, type 4a, type 4c, type 4d, type 4f, type 4h, type 4k, type 5, type 5a, type 6 and type 6a.

The terms “individual,” “host,” “subject,” and “patient” are used interchangeably herein, and refer to a mammal, including, but not limited to, non-human primates (e.g., simians), equines (e.g., horses), and humans.

As used herein, the term “isolated,” in reference to a polypeptide, refers to a polypeptide that is in an environment different from that in which the polypeptide naturally occurs. An isolated polypeptide can be purified. By “purified” is meant a compound of interest (e.g., a polypeptide) has been separated from components that accompany it in nature. “Purified” can also be used to refer to a polypeptide separated from components that can accompany it during production of the polypeptide (e.g., during synthesis in vitro, etc.). In some embodiments, a polypeptide (or a mixture of polypeptides) is substantially pure when the polypeptide (or mixture of polypeptides) is at least 60% or at least 75% by weight free from organic molecules with which it is naturally associated or with which it is associated during production. In some embodiments, the polypeptide is from 30% to 60% pure. In some embodiments, the polypeptide (or mixture of polypeptides) is at least 60%, at least 75%, at least 80%, at least 85%, at least 90%, at least 95%, or at least 99%, by weight, pure. For example, in some cases, an E1 polypeptide, an E1-Fc polypeptide, an E2 polypeptide, or an E2-Fc polypeptide (or a heterodimer comprising such polypeptides) is substantially pure when the E1, E1-Fc, E2, or E2-Fc polypeptide (or a heterodimer of such polypeptides) is at least 60% or at least 75% by weight free from organic molecules with which the polypeptide(s) is naturally associated or with which it is associated during production.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. In some cases, a polynucleotide is RNA. In some cases, a polynucleotide is DNA. A “polynucleotide” includes a nucleic acid that is incorporated into a viral vector or a bacterial vector.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The term “polypeptide” includes glycosylated polypeptides.

The term “heterologous” refers to two components that are defined by structures derived from different sources. For example, where “heterologous” is used in the context of a polypeptide, where the polypeptide includes operably linked amino acid sequences that can be derived from one or more different polypeptides, e.g., amino acid sequences that are not operably linked to the polypeptide in nature.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a HCV E1” includes a plurality of such polypeptides and reference to “the affinity tagged HCV E1/E2 heterodimer” includes reference to one or more affinity tagged HCV E1/E2 heterodimers and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides affinity tagged heterodimeric polypeptides comprising a hepatitis C virus (HCV) E1 polypeptide and an HCV E2 polypeptide, where one or both of the E1 and E2 polypeptides comprises an affinity purification tag that facilitates commercial, scaled-up purification and production of the E1E2 heterodimer for delivery of the HCV E1/E2 heterodimer as a vaccine. The present disclosure provides a method of producing an affinity-tagged E1/E2 heterodimer of the present disclosure. The present disclosure provides a method of producing an (untagged) HCV E1/E2 heterodimer suitable for use as a vaccine.

### Affinity Tagged HCV E1/E2 Heterodimers

The present disclosure provides a method to facilitate large-scale purification of the E1E2 glycoprotein heterodimer using affinity purification tags such as the human Fc immunoglobulin domain fused to the N-terminus of E2, for example. Following purification of the E1E2 heterodimer complex, the human Fc domain can be removed from the E1E2 complex by digestion with a suitable protease at a proteolytic cleavage site placed downstream of the Fc domain. Ordinarily, the large scale purification of E1E2 is very challenging and severely limits its use as a HCV vaccine in humans. The present disclosure provides a method to address this challenge, and to produce HCV E1/E2 heterodimer suitable for use as a vaccine. The present disclosure also provides: a) an HCV E1-affinity tag fusion polypeptide and an HCV E2 polypeptide; b) an HCV E1 polypeptide and an HCV E2-affinity tag fusion polypeptide; or c) an HCV E1-affinity tag fusion polypeptide and an HCV E2-affinity tag fusion polypeptide. The affinity tag portion of an HCV E1-affinity tag fusion polypeptide can be at the N-terminus of the HCV E1 polypeptide, or at the C-terminus of the HCV E1 polypeptide. Likewise, the affinity tag portion of an HCV E2-affinity tag fusion polypeptide can be at the N-terminus of the HCV E2 polypeptide, or at the C-terminus of the HCV E2 polypeptide.

Suitable affinity tags (also referred to herein as “affinity purification tags”) include immunoglobulin (Ig) Fc polypeptides, Protein A, Protein G, Protein L, a hybrid Protein A-Protein G polypeptide, a polypeptide comprising a poly(histidine) tract, an immunoglobulin light chain, and glutathione-S-transferase (GST). Purification can be achieved using Protein A-immobilized columns or Protein G-immobilized columns (in the case of Ig-tagged E1E2); Protein L-immobilized columns (in the case of Ig light chain-tagged E1E2); Ig-immobilized columns (in the case of Protein A-or Protein G-tagged E1E2); Ni-columns (in the case of His-tagged E1E2); or Glutathione-immobilized columns (in the case of Glutathione-S-transferase-tagged E1E2), as examples.

The present disclosure provides heterodimeric polypeptides comprising: a) an HCV E1-Fc fusion polypeptide and an HCV E2 polypeptide; b) an HCV E1 polypeptide and an HCV E2-Fc fusion polypeptide; or c) an HCV E1-Fc fusion polypeptide and an HCV E2-Fc fusion polypeptide. The Fc portion of an HCV E1-Fc fusion polypeptide can be at the N-terminus of the HCV E1 polypeptide, or at the C-terminus of the HCV E2 polypeptide. Likewise, the Fc portion of an HCV E2-Fc fusion polypeptide can be at the N-terminus of the HCV E2 polypeptide, or at the C-terminus of the HCV E2 polypeptide.

The HCV E1 and E2 polypeptides in an affinity tagged HCV E1/E2 heterodimer of the present disclosure can be of the same HCV genotype, or can be of different HCV genotypes.

In some cases, the HCV E1 and E2 polypeptides in an affinity tagged HCV E1/E2 heterodimer of the present disclosure are genotype 1 HCV E1 and E2 polypeptides. In some cases, the HCV E1 and E2 polypeptides in an affinity tagged HCV E1/E2 heterodimer of the present disclosure are genotype 2 HCV E1 and E2 polypeptides. In some cases, the HCV E1 and E2 polypeptides in an affinity tagged HCV E1/E2 heterodimer of the present disclosure are genotype 3 HCV E1 and E2 polypeptides. In some cases, the HCV E1 and E2 polypeptides in an affinity tagged HCV E1/E2 heterodimer of the present disclosure are genotype 4 HCV E1 and E2 polypeptides. In some cases, the HCV E1 and E2 polypeptides in an affinity tagged HCV E1/E2 heterodimer of the present disclosure are genotype 5 HCV E1 and E2 polypeptides. In some cases, the HCV E1 and E2 polypeptides in an affinity tagged HCV E1/E2 heterodimer of the present disclosure are genotype 6 HCV E1 and E2 polypeptides. In some cases, the HCV E1 and E2 polypeptides in an affinity tagged HCV E1/E2 heterodimer of the present disclosure are genotype 7 HCV E1 and E2 polypeptides.

The HCV E1 and E2 polypeptides in an affinity tagged HCV E1/E2 heterodimer of the present disclosure are in some embodiments full-length HCV E1 and E2 polypeptides. The HCV E1 and E2 polypeptides in an affinity tagged HCV E1/E2 heterodimer of the present disclosure are in some embodiments full-length HCV E1 and E2 polypeptides and, other than the affinity tag (Ig Fc polypeptide) and an optional linker, do not include any heterologous polypeptides.

In some cases, an affinity tag can replace the N-terminal 20 to 50 amino acids of an HCV E2 polypeptide. For example, in some cases, an affinity tag can replace the N-terminal 20 amino acids (aa) to 25 aa, from 25 aa to 30 aa, from 30 aa to 35 aa, from 35 aa to 40 aa, or from 40 aa to 50 aa, of an HCV E2 polypeptide. For example, in some cases, an affinity tag can replace the N-terminal 30 amino acids of an HCV E2 polypeptide.

### E2 Polypeptides

An E2 polypeptide suitable for inclusion in an affinity tagged HCV E1/E2 heterodimer of the present disclosure can have a length of from about 200 amino acids (aa) to about 250 aa, from about 250 aa to about 275 aa, from about 275 aa to about 300 aa, from about 300 aa to about 325 aa, from about 325 aa to about 350 aa, or from about 350 aa to about 365 aa. In some cases, an HCV E2 polypeptide suitable for inclusion in an E1/E2 heterodimer of the present disclosure is an HCV E2 ectodomain polypeptide. In some cases, an HCV E2 polypeptide suitable for inclusion in an affinity tagged HCV E1/E2 heterodimer of the present disclosure is a full-length HCV E2 polypeptide.

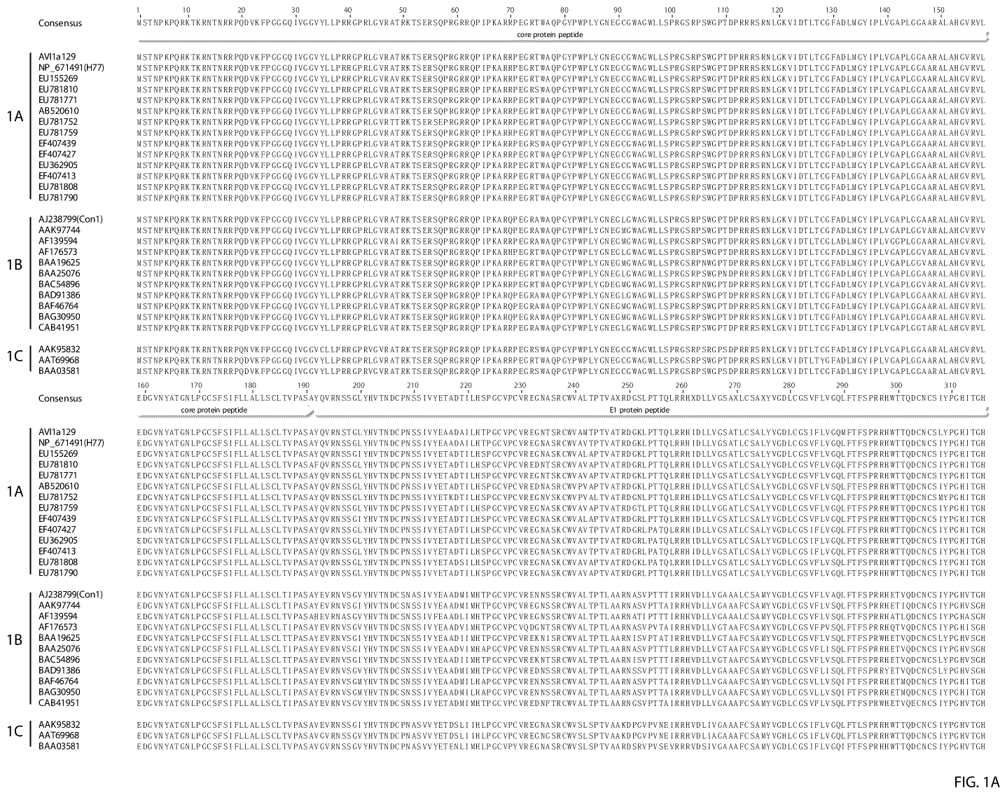

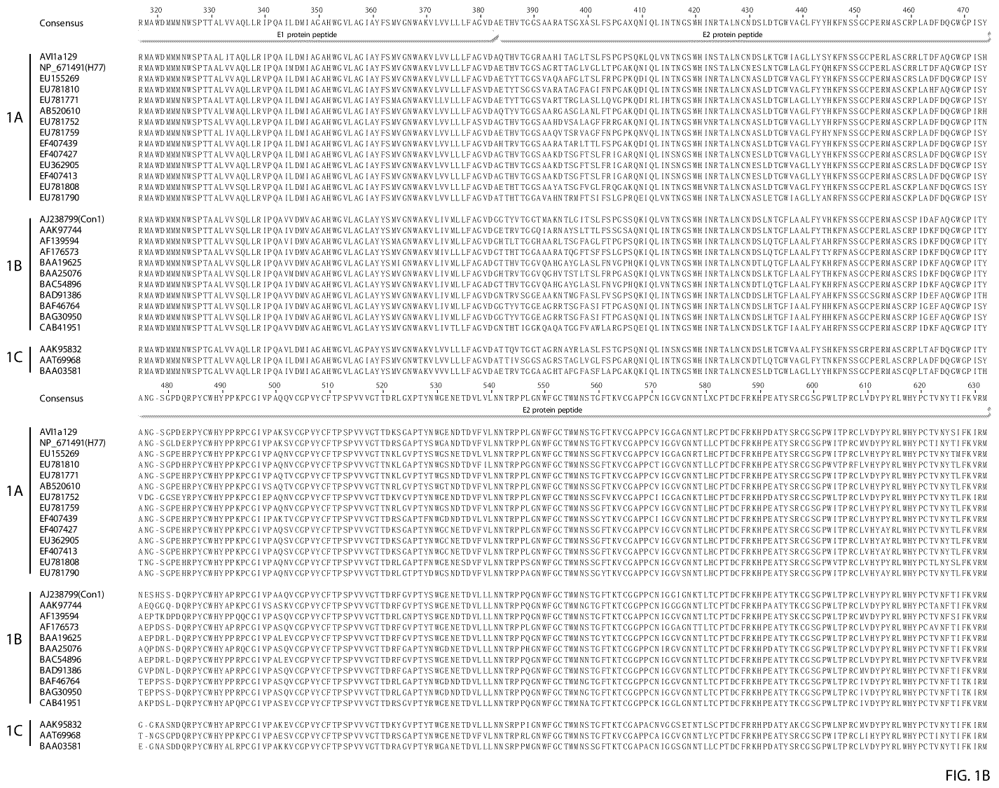

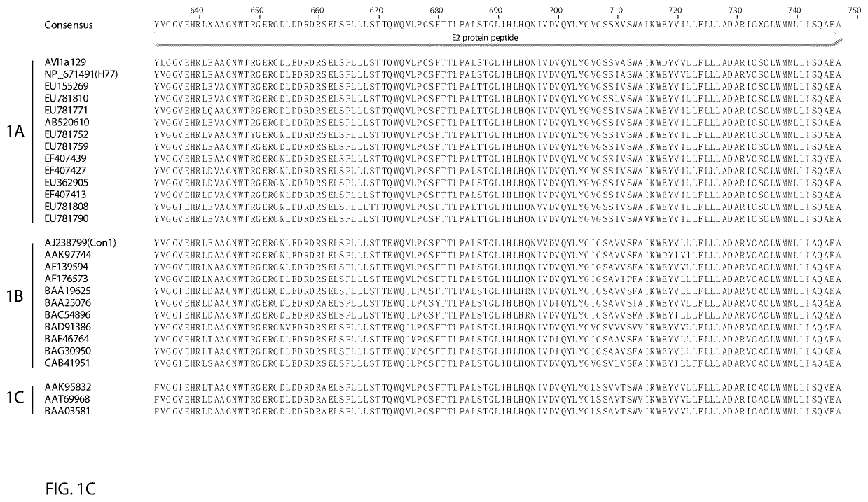

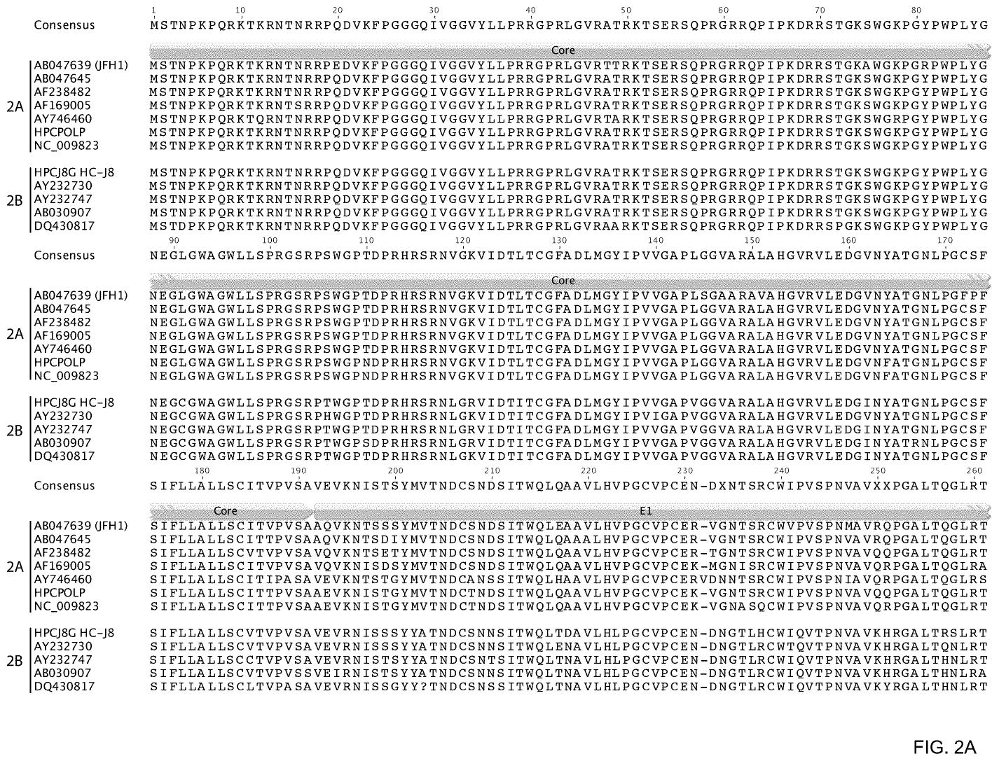

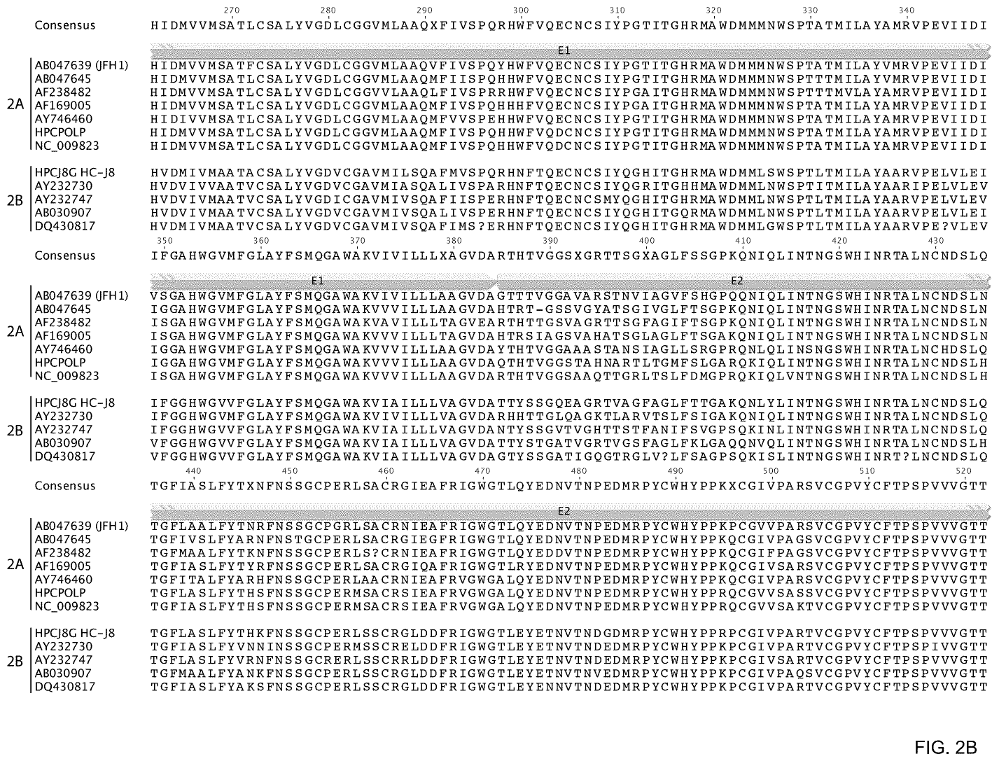

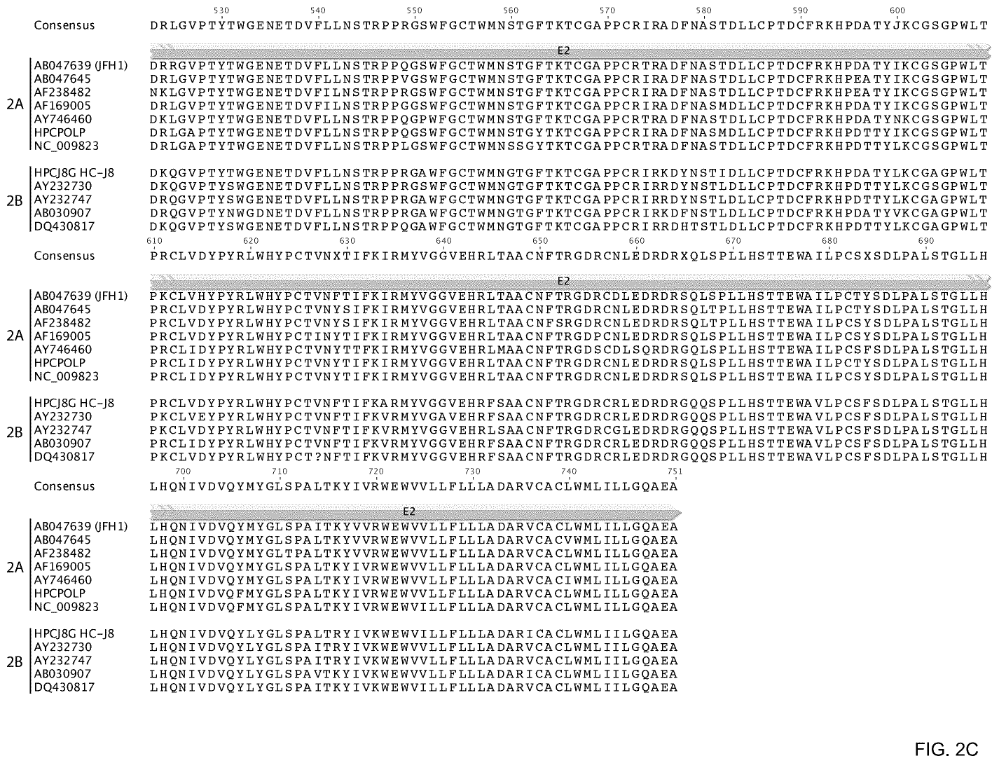

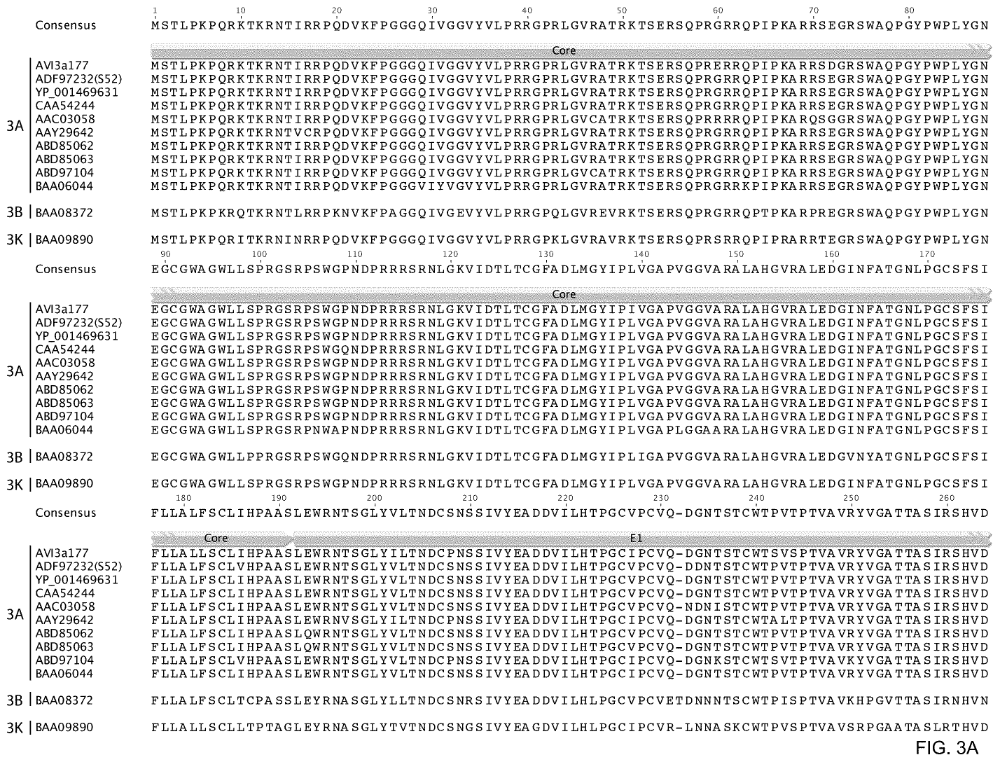

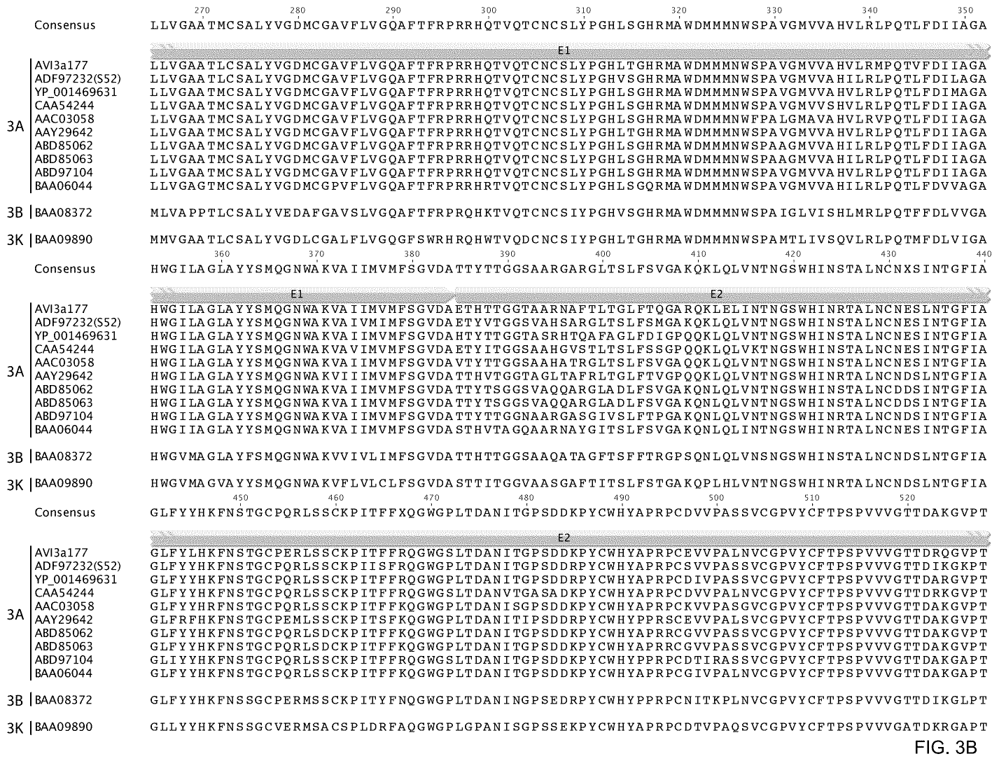

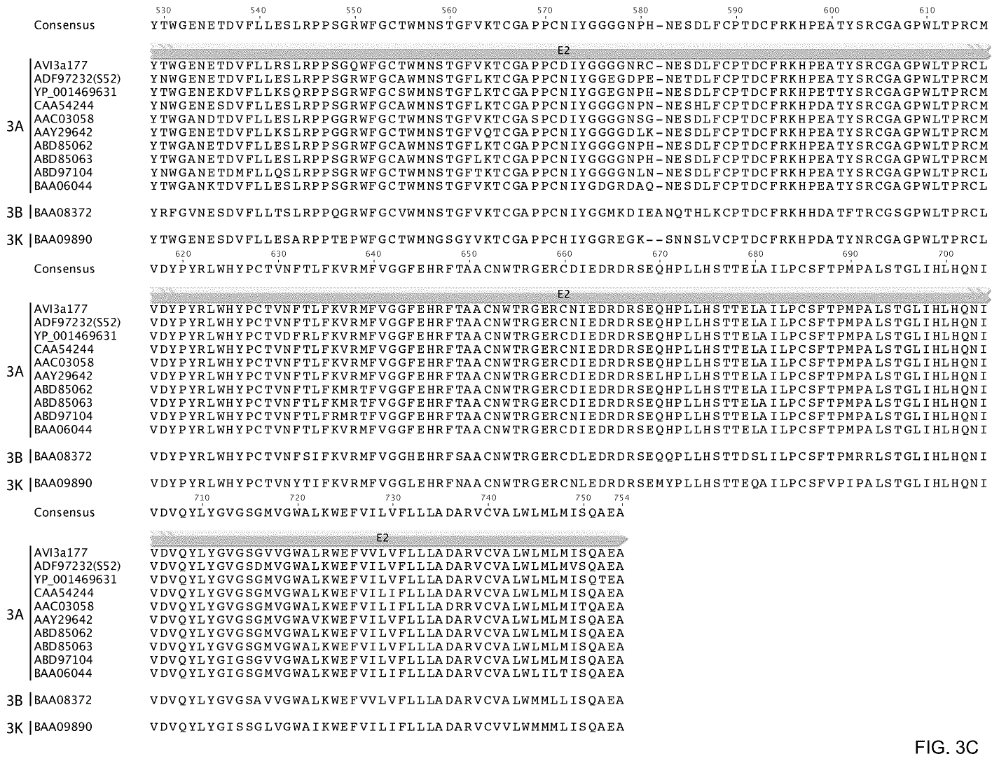

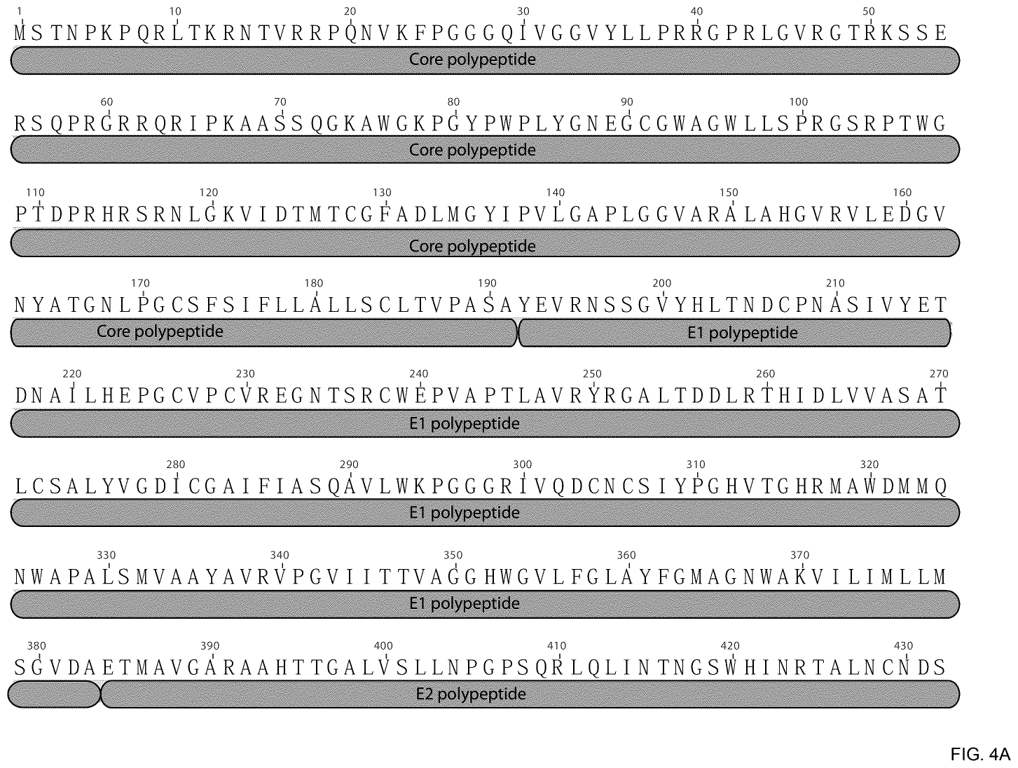

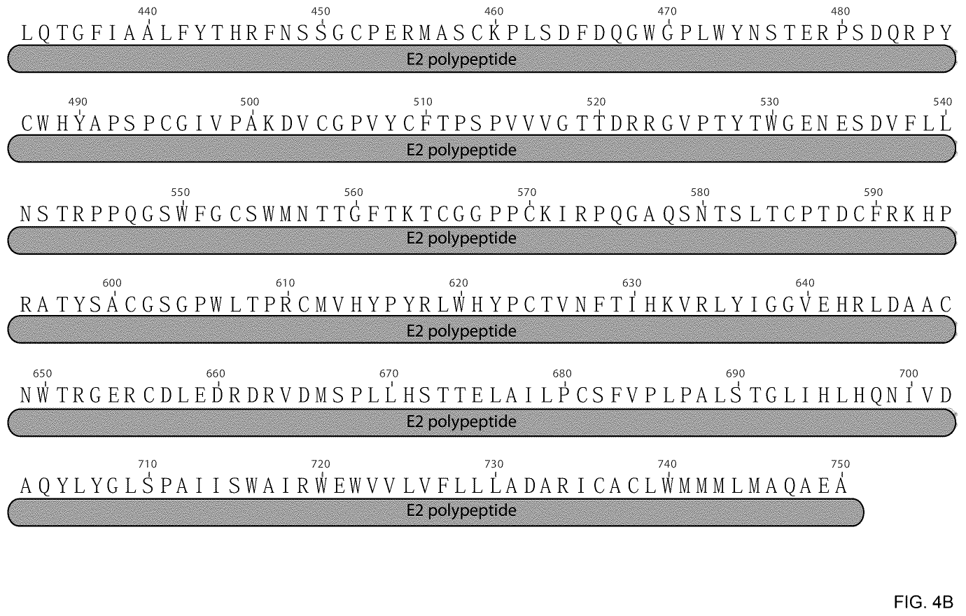

In FIG. 1A-AC, the amino acid sequence of E2 is amino acid 384 to amino acid 746. In FIG. 2A-2B, the amino acid sequence of E2 is amino acid 384 to amino acid 751. In FIG. 3A-3C, the amino acid sequence of E2 is amino acid 385 to amino acid 754. In FIG. 4A-4B, the amino acid sequence of E2 is amino acid 384 to amino acid 750. As used herein, an “E2 polypeptide” includes a precursor E2 protein, including the signal sequence; includes a mature E2 polypeptide which lacks this sequence; and includes an E2 polypeptide with a heterologous signal sequence. An E2 polypeptide can include a C-terminal membrane anchor sequence which occurs at approximately amino acid positions 715-730 and may extend as far as approximately amino acid residue 746 (see, Lin et al., J. Virol. (1994) 68:5063-5073).

In some cases, a E2 polypeptide suitable for inclusion in an affinity tagged HCV E1/E2 heterodimer of the present disclosure lacks a portion of its C-terminal region, e.g., from about amino acid 715 to the C-terminus; from about amino acid 625 to the C-terminus; from about amino acid 661 to the C-terminus; from about amino acid 655 to the C-terminus; from about amino acid 500 to the C-terminus, where the amino acid numbering is with reference to the numbering in FIG. 1A-1C. See, e.g., U.S. Pat. No. 6,521,423.

An E2 polypeptide suitable for inclusion in an affinity tagged HCV E1/E2 heterodimer of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E2 polypeptide depicted in FIG. 1A-1C, FIG. 2A-2C, FIG. 3A-3C, or FIG. 4A-4B. An E2 polypeptide suitable for inclusion in an E1/E2 heterodimer of the present disclosure can comprise an amino acid sequence having at least about 20%, at least about 25%, at least about 30%, at least about 35%, at least about 40%, at least about 45%, at least about 50%, at least about 60%, at least about 70%, or at least about 75%, amino acid sequence identity to an amino acid sequence of an E2 polypeptide depicted in FIG. 1A-1C, FIG. 2A-2C, FIG. 3A-3C, or FIG. 4A-4B.

An E2 polypeptide suitable for inclusion in an affinity tagged HCV E1/E2 heterodimer of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E2 polypeptide depicted in FIG. 1A-1C. For example, an E2 polypeptide of genotype 1 can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-746 of an amino acid sequence depicted in FIG. 1A-1C. For example, an E2 polypeptide of genotype 1A can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-746 of an amino acid sequence identified as 1A and depicted in FIG. 1A-1C. For example, an E2 polypeptide of genotype 1B can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-746 of an amino acid sequence identified as 1B and depicted in FIG. 1A-1C. For example, an E2 polypeptide of genotype 1C can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-746 of an amino acid sequence identified as 1C and depicted in FIG. 1A-1C.

An E2 polypeptide suitable for inclusion in an affinity tagged HCV E1/E2 heterodimer of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E2 polypeptide depicted in FIG. 2A-2C. For example, an E2 polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-751 of an amino acid sequence depicted in FIG. 2A-2C. For example, an E2 polypeptide of genotype 2A can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-751 of the “consensus” amino acid sequence depicted in FIG. 2A-2C. For example, an E2 polypeptide of genotype 2B can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-751 of the “consensus” amino acid sequence depicted in FIG. 2A-2C.

An E2 polypeptide suitable for inclusion in an affinity tagged HCV E1/E2 heterodimer of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E2 polypeptide depicted in FIG. 3A-3C. For example, an E2 polypeptide of genotype 3 can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 385-754 of an amino acid sequence depicted in FIG. 3A-3C. For example, an E2 polypeptide of genotype 3A can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 385-754 of an amino acid sequence identified as 3A and depicted in FIG. 3A-3C. For example, an E2 polypeptide of genotype 3B can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 385-754 of the amino acid sequence identified as 3B and depicted in FIG. 3A-3C. For example, an E2 polypeptide of genotype 3K can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 385-754 of the amino acid sequence identified as 3K and depicted in FIG. 3A-3C.

An E2 polypeptide suitable for inclusion in an affinity tagged HCV E1/E2 heterodimer of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the amino acid sequence of the E2 polypeptide depicted in FIG. 4A-4B. For example, an E2 polypeptide of genotype 7A can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-750 of the amino acid sequence depicted in FIG. 4A-4B.

### E1 Polypeptides

An HCV E1 polypeptide suitable for inclusion in an affinity tagged HCV E1/E2 heterodimer of the present disclosure can have a length of from about 150 amino acids (aa) to about 175 aa, from about 175 aa to about 195 aa, from about 131 aa to about 175 aa, or from about 175 aa to about 193 aa. In some cases, an HCV E1 polypeptide suitable for inclusion in an E1/E2 heterodimer of the present disclosure is an HCV E1 ectodomain polypeptide. In some cases, an HCV E1 polypeptide suitable for inclusion in an E1/E2 heterodimer of the present disclosure is a full-length HCV E1 polypeptide.

In FIG. 1A-1C, the amino acid sequence of E1 is amino acid 192 to amino acid 383. In FIG. 2A-2C, the amino acid sequence of E1 is amino acid 192 to amino acid 383. In FIG. 3A-3C, the amino acid sequence of E1 is amino acid 192 to amino acid 384. In FIG. 4A-4B, the amino acid sequence of E1 is amino acid 192 to amino acid 383. Amino acids at around 170 through approximately 191 serve as a signal sequence for E1. As used herein, “E1 polypeptide” includes a precursor E1 protein, including the signal sequence; includes a mature E1 polypeptide which lacks this sequence; and includes an E1 polypeptide with a heterologous signal sequence. An E1 polypeptide can include a C-terminal membrane anchor sequence which occurs at approximately amino acid positions 360-383 (see, e.g., WO 96/04301). In some cases, a suitable E1 polypeptide lacks a C-terminal portion that includes a transmembrane region. For example, in some cases, a suitable E1 polypeptide lacks the C-terminal portion from amino acid 330 to amino acid 384, or from amino acid 360 to amino acid 384. E1 polypeptides can be an E1 polypeptide of any genotype, subtype or isolate of HCV. E1 polypeptides of genotype 1 and E1 polypeptides of genotype 3 are included in an E1/E2 heterodimer of the present disclosure.

An E1 polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E1 polypeptide depicted in FIG. 1A-1C, FIG. 2A-2C, FIG. 3A-3C, or FIG. 4A-4B.

An E1 polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E1 polypeptide depicted in FIG. 1A-1C. For example, an E1 polypeptide of genotype 1A can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 192-383 of an amino acid sequence identified as 1A and depicted in FIG. 1A-1C. For example, an E1 polypeptide of genotype 1B can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 192-383 of an amino acid sequence identified as 1B and depicted in FIG. 1A-1C. For example, an E1 polypeptide of genotype 1C can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 192-383 of an amino acid sequence identified as 1C and depicted in FIG. 1A-1C.

An E1 polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E1 polypeptide depicted in FIG. 2A-2C. For example, an E1 polypeptide of genotype 2A can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 192-383 of an amino acid sequence identified as 2A and depicted in FIG. 2A-2C. For example, an E1 polypeptide of genotype 2B can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 192-383 of an amino acid sequence identified as 2B and depicted in FIG. 2A-2C.

An E1 polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the consensus E1 polypeptide amino acid sequence depicted in FIG. 3A-3C. For example, an E1 polypeptide of genotype 3A, 3B, or 3K can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino 192-384 of an amino acid sequence of an amino acid sequence identified as 3A, 3B, or 3K, respectively, as depicted in FIG. 3A-3C.

An E1 polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E1 polypeptide depicted in FIG. 4A-4B. For example, an E1 polypeptide of genotype 7A can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 192-383 of the amino acid sequence depicted in FIG. 4A-4B.

### Affinity Tags

Suitable affinity tags (also referred to herein as “affinity purification tags”) include immunoglobulin (Ig) Fc polypeptides, Protein A, Protein G, Protein L, a hybrid Protein A-Protein G polypeptide, a polypeptide comprising a poly(histidine) tract, an immunoglobulin light chain, and glutathione-S-transferase (GST).

### Fc Polypeptides

Various Ig Fc polypeptides are suitable for inclusion in an affinity tagged HCV E1/E2 heterodimer of the present disclosure. The Fc region (Fc polypeptide) can be a human IgG1 Fc, a human IgG2 Fc, a human IgG3 Fc, a human IgG4 Fc, etc. The Fc polypeptide can be an Fc polypeptide from a non-human species.

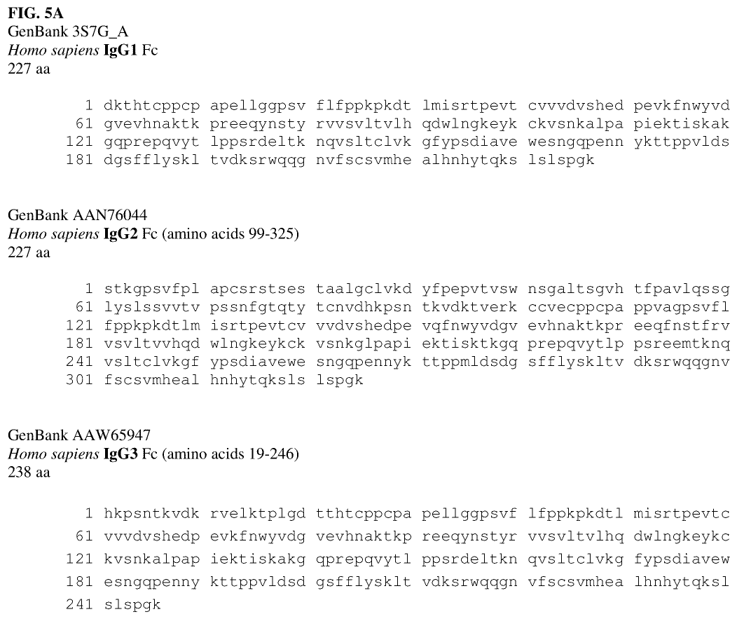

In some cases, the Fc region comprises an amino acid sequence having at least about 70%, at least about 75%, at least about 80%, at least about 85%, at least about 90%, at least about 95%, at least about 98%, at least about 99%, or 100%, amino acid sequence identity to an amino acid sequence of an Fc region depicted in FIG. 5A-5C. In some cases, the Fc region comprises an amino acid sequence having at least about 70%, at least about 75%, at least about 80%, at least about 85%, at least about 90%, at least about 95%, at least about 98%, at least about 99%, or 100%, amino acid sequence identity to the human IgG1 Fc polypeptide depicted in FIG. 5A. In some cases, the Fc region comprises an amino acid sequence having at least about 70%, at least about 75%, at least about 80%, at least about 85%, at least about 90%, at least about 95%, at least about 98%, at least about 99%, or 100%, amino acid sequence identity to the human IgG2 Fc polypeptide depicted in FIG. 5A; e.g., the Fc region comprises an amino acid sequence having at least about 70%, at least about 75%, at least about 80%, at least about 85%, at least about 90%, at least about 95%, at least about 98%, at least about 99%, or 100%, amino acid sequence identity to amino acids 99-325 of the human IgG2 Fc polypeptide depicted in FIG. 5A. In some cases, the Fc region comprises an amino acid sequence having at least about 70%, at least about 75%, at least about 80%, at least about 85%, at least about 90%, at least about 95%, at least about 98%, at least about 99%, or 100%, amino acid sequence identity to the human IgG3 Fc polypeptide depicted in FIG. 5A; e.g., the Fc region comprises an amino acid sequence having at least about 70%, at least about 75%, at least about 80%, at least about 85%, at least about 90%, at least about 95%, at least about 98%, at least about 99%, or 100%, amino acid sequence identity to amino acids 19-246 of the human IgG3 Fc polypeptide depicted in FIG. 5A.

In some cases, the Fc region comprises an amino acid sequence having at least about 70%, at least about 75%, at least about 80%, at least about 85%, at least about 90%, at least about 95%, at least about 98%, at least about 99%, or 100%, amino acid sequence identity to the human IgG4 Fc polypeptide depicted in FIG. 5C; e.g., the Fc region comprises an amino acid sequence having at least about 70%, at least about 75%, at least about 80%, at least about 85%, at least about 90%, at least about 95%, at least about 98%, at least about 99%, or 100%, amino acid sequence identity to amino acids 100-327 of the human IgG4 Fc polypeptide depicted in FIG. 5C.

### Protein A

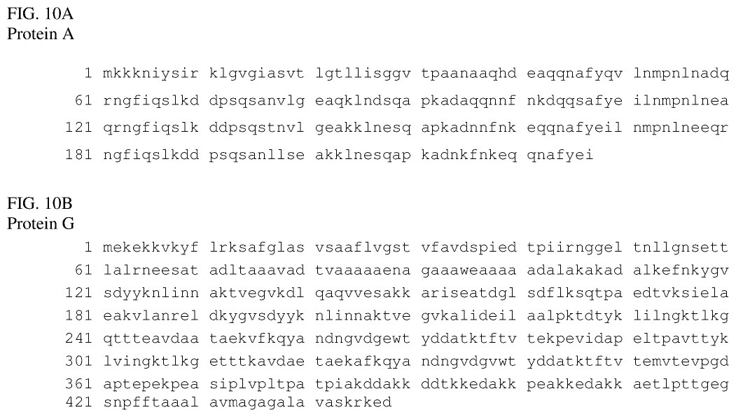

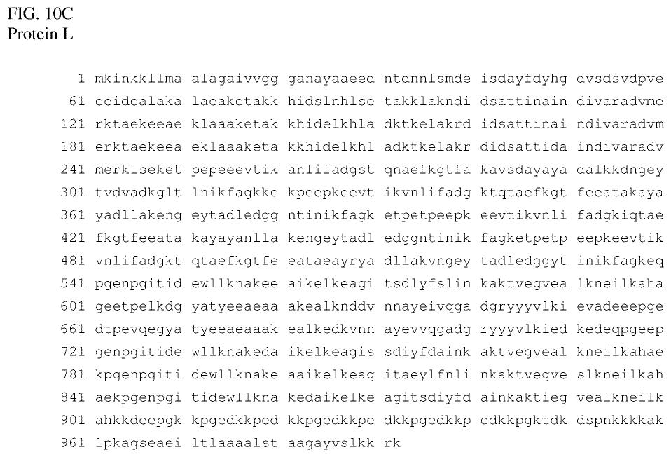

In some cases, the affinity tag polypeptide included in an affinity tagged HCV E1/E2 heterodimer of the present disclosure is a Protein A polypeptide. A suitable Protein A polypeptide can comprises an amino acid sequence having at least 50%, at least 60%, at least 70%, at least 80%, at least 85%, at least 90%, at least 95%, at least 98%, or 100%, amino acid sequence identity to amino acids 27 to 227 the Protein A amino acid sequence depicted in FIG. 10A; and can have a length of from 150 to 200 amino acids. A suitable Protein A polypeptide can comprises an amino acid sequence having at least 50%, at least 60%, at least 70%, at least 80%, at least 85%, at least 90%, at least 95%, at least 98%, or 100%, amino acid sequence identity to amino acids 27 to 227 the Protein A amino acid sequence depicted in FIG. 10A; and can have a length of 200 amino acids.

### Protein G

In some cases, the affinity tag polypeptide included in an affinity tagged HCV E1/E2 heterodimer of the present disclosure is a Protein G polypeptide. A suitable Protein G polypeptide can comprises an amino acid sequence having at least 50%, at least 60%, at least 70%, at least 80%, at least 85%, at least 90%, at least 95%, at least 98%, or 100%, amino acid sequence identity to amino acids 37-448 the Protein G amino acid sequence depicted in FIG. 10B; and can have a length of from 350 amino acids to 411 amino acids.

### Protein L

Protein L binds antibody kappa light chains, single chain variable fragments (scFv), and Fab fragments. A suitable Protein L polypeptide can comprises an amino acid sequence having at least 50%, at least 60%, at least 70%, at least 80%, at least 85%, at least 90%, at least 95%, at least 98%, or 100%, amino acid sequence identity to amino acids 25-992 of the Protein L amino acid sequence depicted in FIG. 10C. A suitable Protein L polypeptide can comprises an amino acid sequence having at least 50%, at least 60%, at least 70%, at least 80%, at least 85%, at least 90%, at least 95%, at least 98%, or 100%, amino acid sequence identity to amino acids 25-992 of the Protein L amino acid sequence depicted in FIG. 10C; and can have a length of from 800 amino acids to 968 amino acids.

### Polypeptides Comprising a Poly(Histidine) Tract

In some cases, the affinity tag polypeptide included in an affinity tagged HCV E1/E2 heterodimer of the present disclosure is a polypeptide that comprises a poly(histidine) tract. A suitable poly(histidine) tract-containing polypeptide can have a length of from about 25 amino acids to about 500 amino acids, e.g., from about 25 amino acids (aa) to 30 aa, from 30 aa to 35 aa, from 35 aa to 40 aa, from 40 aa to 50 aa, from 50 aa to 75 aa, from 75 aa to 100 aa, from 100 aa to 150 aa, from 150 aa to 200 aa, from 200 aa to 250 aa, from 250 aa to 300 aa, from 300 aa to 350 aa, from 350 aa to 400 aa, from 400 aa to 450 aa, or from 450 aa to 500 aa. The poly(histidine) tract can be from 4 to 20 consecutive histidines, e.g., from 4 to 6 consecutive histidines, from 6 to 10 consecutive histidines, from 10 to 15 consecutive histidines, or from 15 to 20 consecutive histidines. For example, a suitable affinity tag polypeptide can have a length of about 50 amino acids, and can include a (His) 6 histidine tract.

### Light Chain

In some cases, the affinity tag polypeptide included in an affinity tagged HCV E1/E2 heterodimer of the present disclosure is an immunoglobulin light chain, or an Ig light chain-containing polypeptide. For example, an Ig light chain-containing polypeptide can be a single chain variable fragment (scFv), an Fab fragment, or any other Ig light chain-containing polypeptide. In some cases, the Ig light chain-containing polypeptide comprises an Ig kappa light chain, e.g., a human Ig kappa light chain. Ig light chain amino acid sequences are known in the art.

### Linkers

In some cases, a linker can be interposed between the affinity tag (e.g., Ig Fc) and the HCV E1 or HCV E2 polypeptide. The linker peptide may have any of a variety of amino acid sequences. A linker can be a peptide of between about 6 and about 40 amino acids in length, or between about 6 and about 25 amino acids in length. These linkers can be produced by using synthetic, linker-encoding oligonucleotides to couple the proteins. Peptide linkers allowing a degree of flexibility can be used. The linking peptides may have virtually any amino acid sequence, bearing in mind that suitable linkers will have a sequence that results in a generally flexible peptide. The use of small amino acids, such as glycine and alanine, are of use in creating a flexible peptide. The creation of such sequences is routine to those of skill in the art.

Suitable linkers can be readily selected and can be of any of a suitable of different lengths, such as from 1 amino acid (e.g., Gly, Ala, or Ser) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Exemplary flexible linkers include glycine polymers (G) n, glycine-serine polymers (including, for example, (GS) n, (GSGGS), (SEQ ID NO:9) and (GGGS), (SEQ ID NO:10), where n is an integer of at least one), glycine-alanine polymers, alanine-serine polymers, and other flexible linkers known in the art. Glycine and glycine-serine polymers are of interest since both of these amino acids are relatively unstructured, and therefore may serve as a neutral tether between components. Glycine polymers are of particular interest since glycine accesses significantly more phi-psi space than even alanine, and is much less restricted than residues with longer side chains (see Scheraga, Rev. Computational Chem. 11173-142 (1992)). Exemplary flexible linkers include, but are not limited to, GGSG (SEQ ID NO:11), GGSGG (SEQ ID NO:12), GSGSG (SEQ ID NO:13), GSGGG (SEQ ID NO:14), GGGSG (SEQ ID NO:15), GSSSG (SEQ ID NO: 16), and the like. The ordinarily skilled artisan will recognize that design of a peptide conjugated to any elements described above can include linkers that are all or partially flexible, such that the linker can include a flexible linker as well as one or more portions that confer less flexible structure.

### E1-Fc Fusion/E2 Heterodimers

In some cases, an affinity tagged HCV E1/E2 heterodimer of the present disclosure comprises: a) an HCV E1-Fc fusion polypeptide; and b) an HCV E2 polypeptide.

The Fc polypeptide can be attached, directly or via a linker, to the carboxyl terminus (C terminus) of an HCV E1 polypeptide. In other words, in some cases, the HCV E1-Fc fusion polypeptide comprises, in order from N-terminus to C-terminus: i) an HCV E1 polypeptide; and ii) an Ig Fc polypeptide. In some case, the HCV E1-Fc fusion polypeptide comprises, in order from N-terminus to C-terminus: i) an HCV E1 polypeptide; ii) a linker; and iii) an Ig Fc polypeptide.

The Fc polypeptide can be attached, directly or via a linker, to the amino terminus (N terminus) of the HCV E1 polypeptide. In other words, in some cases, the HCV E1-Fc fusion polypeptide comprises, in order from N-terminus to C-terminus: i) an Ig Fc polypeptide; and ii) an HCV E1 polypeptide. In some cases, the HCV E1-Fc fusion polypeptide comprises, in order from N-terminus to C-terminus: i) an Ig Fc polypeptide; ii) a linker; and iii) an HCV E1 polypeptide

### E1/E2-Fc Fusion Heterodimers

In some cases, an affinity tagged HCV E1/E2 heterodimer of the present disclosure comprises: a) an HCV E2-Fc fusion polypeptide; and b) an HCV E1 polypeptide.

The Fc polypeptide can be attached, directly or via a linker, to the C-terminus of an HCV E2 polypeptide. In other words, in some cases, the HCV E2-Fc fusion polypeptide comprises, in order from N-terminus to C-terminus: i) an HCV E2 polypeptide; and ii) an Ig Fc polypeptide. In some cases, the HCV E2-Fc fusion polypeptide comprises, in order from N-terminus to C-terminus: i) an HCV E2 polypeptide; ii) a linker; and iii) an Ig Fc polypeptide.

The Fc polypeptide can be attached, directly or via a linker, to N-terminus of the HCV E2 polypeptide. In other words, in some cases, the HCV E2-Fc fusion polypeptide comprises, in order from N-terminus to C-terminus: i) an Ig Fc polypeptide; and ii) an HCV E2 polypeptide. In some cases, the HCV E2-Fc fusion polypeptide comprises, in order from N-terminus to C-terminus: i) an Ig Fc polypeptide; ii) a linker; and iii) an HCV E2 polypeptide.

### E1-Fc Fusion/E2-Fc Fusion Heterodimers

In some cases, an affinity tagged HCV E1/E2 heterodimer of the present disclosure comprises: a) an HCV E1-Fc fusion polypeptide; and b) an HCV E2-Fc fusion polypeptide.

In some cases, an affinity tagged HCV E1/E2 heterodimer of the present disclosure comprises: a) an HCV E1-Fc fusion polypeptide, where the HCV E1-Fc fusion polypeptide comprises, in order from N-terminus to C-terminus: i) an HCV E1 polypeptide; and ii) an Fc polypeptide; and b) an HCV E2-Fc fusion polypeptide, where the HCV E2-Fc fusion polypeptide comprises, in order from N-terminus to C-terminus: i) an HCV E2 polypeptide; and ii) an Fc polypeptide.

In some cases, an affinity tagged HCV E1/E2 heterodimer of the present disclosure comprises: a) an HCV E1-Fc fusion polypeptide, where the HCV E1-Fc fusion polypeptide comprises, in order from N-terminus to C-terminus: i) an Fc polypeptide; and ii) an HCV E1 polypeptide; and b) an HCV E2-Fc fusion polypeptide, where the HCV E2-Fc fusion polypeptide comprises, in order from N-terminus to C-terminus: i) an Fc polypeptide; and ii) an HCV E2 polypeptide.

### Proteolytically Cleavable Linker

The affinity tag present in an affinity tagged HCV E1/E2 heterodimer of the present disclosure is cleaved off during purification, to generate an HCV E1/E2 heterodimer that does not include the affinity tag. To achieve this aim, a proteolytically cleavable linker can be positioned between an affinity tag and an HCV E1 and/or HCV E2 polypeptide.

Thus, in some cases, an affinity tagged HCV E1/E2 heterodimer of the present disclosure comprises an affinity tagged HCV E1 polypeptide, where the affinity tagged E1 polypeptide comprises, in order from N-terminus to C-terminus: i) an affinity tag (e.g., an Fc polypeptide); ii) a proteolytically cleavable linker; and ii) an HCV E1 polypeptide.

In some cases, an affinity tagged HCV E1/E2 heterodimer of the present disclosure comprises an affinity tagged HCV E2 polypeptide, where the affinity tagged E2 polypeptide comprises, in order from N-terminus to C-terminus: i) an affinity tag (e.g., an Fc polypeptide); ii) a proteolytically cleavable linker; and ii) an HCV E2 polypeptide.

In some cases, an affinity tagged HCV E1/E2 heterodimer of the present disclosure comprises an affinity tagged HCV E1 polypeptide and an affinity tagged HCV E2 polypeptide, where the affinity tagged E1 polypeptide comprises, in order from N-terminus to C-terminus: i) an affinity tag (e.g., an Fc polypeptide); ii) a proteolytically cleavable linker; and ii) an HCV E1 polypeptide; and where the affinity tagged E2 polypeptide comprises, in order from N-terminus to C-terminus: i) an affinity tag (e.g., an Fc polypeptide); ii) a proteolytically cleavable linker; and ii) an HCV E2 polypeptide.

The proteolytically cleavable linker can include a protease recognition sequence recognized by a protease selected from the group consisting of alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase, gastricsin, gelatinase, Gly-X carboxypeptidase, glycyl endopeptidase, human rhinovirus 3C protease, hypodermin C, IgA-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin, tissue kallikrein, tobacco etch virus (TEV), togavirin, tryptophanyl aminopeptidase, U-plasminogen activator, V8, venombin A, venombin AB, and Xaa-pro aminopeptidase.

For example, the proteolytically cleavable linker can comprise a matrix metalloproteinase (MMP) cleavage site, e.g., a cleavage site for a MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). For example, the cleavage sequence of MMP-9 is Pro-X-X-Hy (wherein, X represents an arbitrary residue; Hy, a hydrophobic residue), e.g., Pro-X-X-Hy-(Ser/Thr), e.g., Pro-Leu/Gln-Gly-Met-Thr-Ser (SEQ ID NO:17) or Pro-Leu/Gln-Gly-Met-Thr (SEQ ID NO:18). Another example of a protease cleavage site is a plasminogen activator cleavage site, e.g., a uPA or a tissue plasminogen activator (tPA) cleavage site. Another example of a suitable protease cleavage site is a prolactin cleavage site. Specific examples of cleavage sequences of uPA and tPA include sequences comprising Val-Gly-Arg. Another example of a protease cleavage site that can be included in a proteolytically cleavable linker is a tobacco etch virus (TEV) protease cleavage site, e.g., ENLYFQS (SEQ ID NO:2), where the protease cleaves between the glutamine and the serine. Another example of a protease cleavage site that can be included in a proteolytically cleavable linker is an enterokinase cleavage site, e.g., DDDDK (SEQ ID NO:3), where cleavage occurs after the lysine residue. Another example of a protease cleavage site that can be included in a proteolytically cleavable linker is a thrombin cleavage site, e.g., LVPR (SEQ ID NO:4) (e.g., where the proteolytically cleavable linker comprises the sequence LVPRGS (SEQ ID NO:5)). Additional suitable linkers comprising protease cleavage sites include linkers comprising one or more of the following amino acid sequences: LEVLFQGP (SEQ ID NO:1), cleaved by PreScission protease (a fusion protein comprising human rhinovirus 3C protease and glutathione-S-transferase; Walker et al. (1994) Biotechnol. 12:601); a thrombin cleavage site, e.g., CGLVPAGSGP (SEQ ID NO:19); SLLKSRMVPNFN (SEQ ID NO:20) or SLLIARRMPNFN (SEQ ID NO:21), cleaved by cathepsin B; SKLVQASASGVN (SEQ ID NO: 22) or SSYLKASDAPDN (SEQ ID NO:23), cleaved by an Epstein-Barr virus protease; RPKPQQFFGLMN (SEQ ID NO:24) cleaved by MMP-3 (stromelysin); SLRPLALWRSFN (SEQ ID NO:25) cleaved by MMP-7 (matrilysin); SPQGIAGQRNFN (SEQ ID NO:26) cleaved by MMP-9; DVDERDVRGFASFL SEQ ID NO:27) cleaved by a thermolysin-like MMP; SLPLGLWAPNEN (SEQ ID NO:28) cleaved by matrix metalloproteinase 2 (MMP-2); SLLIFRSWANFN (SEQ ID NO:29) cleaved by cathespin L; SGVVIATVIVIT (SEQ ID NO:30) cleaved by cathepsin D; SLGPQGIWGQFN (SEQ ID NO:31) cleaved by matrix metalloproteinase 1 (MMP-1); KKSPGRVVGGSV (SEQ ID NO:32) cleaved by urokinase-type plasminogen activator; PQGLLGAPGILG (SEQ ID NO:33) cleaved by membrane type 1 matrixmetalloproteinase (MT-MMP); HGPEGLRVGFYESDVMGRGHARLVHVEEPHT (SEQ ID NO: 34) cleaved by stromelysin 3 (or MMP-11), thermolysin, fibroblast collagenase and stromelysin-1; GPQGLAGQRGIV (SEQ ID NO:35) cleaved by matrix metalloproteinase 13 (collagenase-3); GGSGQRGRKALE (SEQ ID NO:36) cleaved by tissue-type plasminogen activator (tPA); SLSALLSSDIFN (SEQ ID NO:37) cleaved by human prostate-specific antigen; SLPRFKIIGGFN (SEQ ID NO:38) cleaved by kallikrein (hK3); SLLGIAVPGNFN (SEQ ID NO: 39) cleaved by neutrophil elastase; and FFKNIVTPRTPP (SEQ ID NO:40) cleaved by calpain (calcium activated neutral protease).

### E1-E2 Junctions

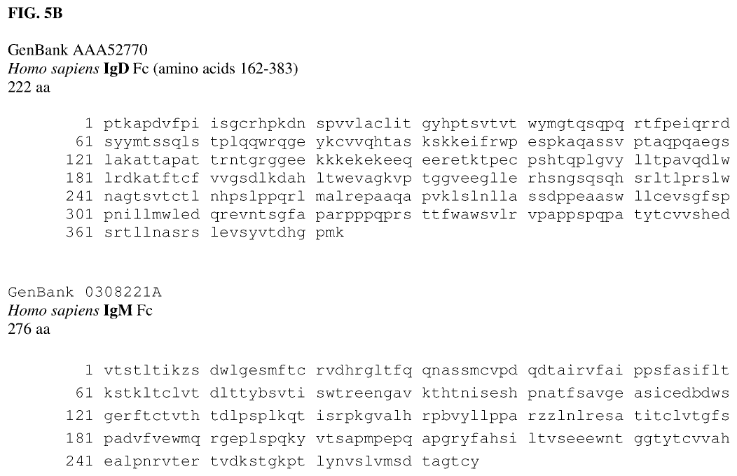

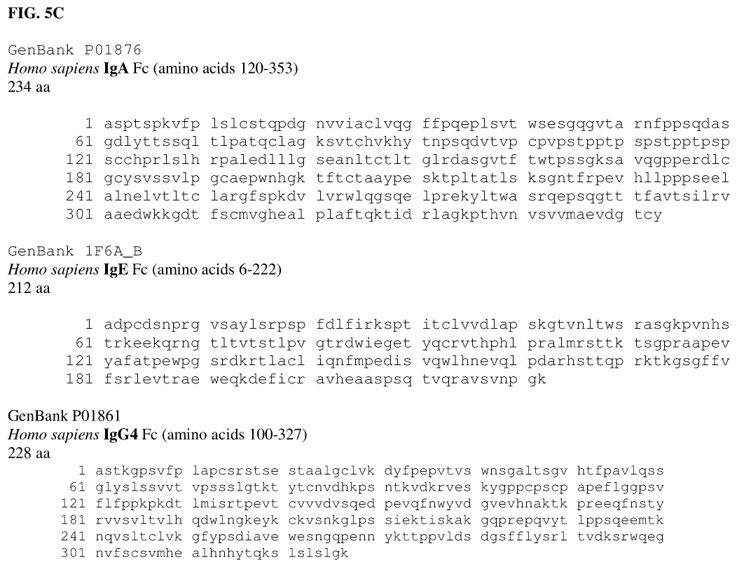

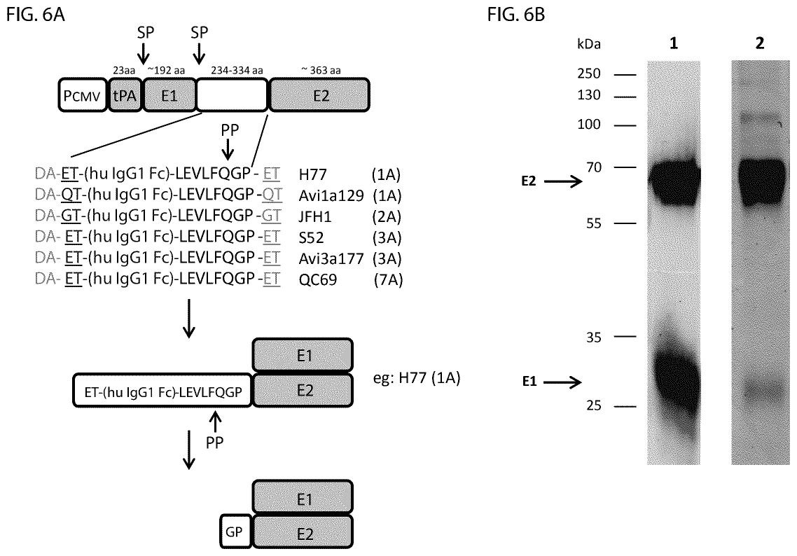

During processing of a wild-type HCV E1-E2 polyprotein in a eukaryotic (e.g., mammalian) cell, endogenous proteases cleave the E1 protein from the E2 protein; the endogenous proteases recognize the E1-E2 junction and cleave at the junction. During synthesis and processing of an affinity tagged HCV E1/E2 heterodimer of the present disclosure from a polyprotein precursor in a mammalian cell, the wild-type E1-E2 junction can be recapitulated, such that amino acids (e.g., from 2 to 15 amino acids (aa) (e.g., 2 aa, 3 aa, 4 aa, 5 aa, 6 aa, 7 aa, 8 aa, 9 aa, 10 aa, 11 aa, 12 aa, 13 aa, 14 aa, or 15 aa)) from the N-terminus of E2 are repeated at the N-terminus of the affinity tag, as depicted in FIG. 6A. In some cases, 2 amino acids from the N-terminus of an HCV E2 polypeptide are positioned at the N-terminus of the affinity tag. In some cases, the dipeptide QT, ET, HT, GT, TT, RH, NT, AY, VI, or ST is positioned at the N-terminus of the affinity tag. Depending on the genotype and specific isolate of E1E2, duplication of the first two amino acids of E2 may result in an undesirable amino acid created at the N-terminus of E2 following processing by signal peptidase (SP) (FIG. 6A). Such amino acids at the amino terminus include asparagine (N), glutamine (Q) or cysteine (C). Such amino acids can target the protein for proteasome-mediated degradation via the N-end rule pathway (reviewed in: Tasaki T et al. 2012. Annu Rev Biochem 81 261-289). In this case, an alternative amino acid could be selected according to either the consensus sequence for the particular genotype or a particular genotype subclass would be selected.

Thus, in some cases, an affinity tagged HCV E1/E2 heterodimer of the present disclosure comprises an affinity tagged HCV E2 polypeptide that comprises, in order from N-terminus to C-terminus: i) from 2 to 15 amino acids (e.g., 2 amino acids (aa), 3 aa, 4 aa, 5 aa, 6 aa, 7 aa, 8 aa, 9 aa, 10 aa, 11 aa, 12 aa, 13 aa, 14 aa, or 15 aa) of the N-terminus of an E2 polypeptide; ii) an affinity tag polypeptide; iii) a proteolytically cleavable linker; and iv) an HCV E2 polypeptide.

For example, in some cases, an affinity tagged HCV E1/E2 heterodimer of the present disclosure comprises an affinity tagged HCV E2 polypeptide that comprises, in order from N-terminus to C-terminus: i) ET (Glu-Thr); ii) an affinity tag polypeptide; iii) a proteolytically cleavable linker; and iv) an HCV E2 polypeptide.

As another example, in some cases, an affinity tagged HCV E1/E2 heterodimer of the present disclosure comprises an affinity tagged HCV E2 polypeptide that comprises, in order from N-terminus to C-terminus: i) GT (Gly-Thr); ii) an affinity tag polypeptide; iii) a proteolytically cleavable linker; and iv) an HCV E2 polypeptide.

As another example, in some cases, an affinity tagged HCV E1/E2 heterodimer of the present disclosure comprises an affinity tagged HCV E2 polypeptide that comprises, in order from N-terminus to C-terminus: i) TT (Thr-Thr); ii) an affinity tag polypeptide; iii) a proteolytically cleavable linker; and iv) an HCV E2 polypeptide.

As another example, in some cases, an affinity tagged HCV E1/E2 heterodimer of the present disclosure comprises an affinity tagged HCV E2 polypeptide that comprises, in order from N-terminus to C-terminus: i) a QT, ET, HT, GT, TT, RH, NT, AY, VI, or ST dipeptide; ii) an affinity tag polypeptide; iii) the cleavable linker LEVLFQGP (SEQ ID NO:1); and iv) an HCV E2 polypeptide.

As another example, in some cases, an affinity tagged HCV E1/E2 heterodimer of the present disclosure comprises an affinity tagged HCV E2 polypeptide that comprises, in order from N-terminus to C-terminus: i) a QT, ET, HT, GT, TT, RH, NT, AY, VI, or ST dipeptide; ii) an Ig Fc polypeptide; iii) the cleavable linker LEVLFQGP (SEQ ID NO:1); and iv) an HCV E2 polypeptide.

### Nucleic Acids, Recombinant Expression Vectors, and Host Cells

The present disclosure provides nucleic acids comprising nucleotide sequences encoding an affinity tagged HCV E1/E2 heterodimer of the present disclosure, and recombinant expression vectors comprising the nucleic acids. Thus, the present disclosure provides recombinant expression vectors comprising nucleotide sequences encoding an affinity tagged HCV E1/E2 heterodimer of the present disclosure. The present disclosure provides host cells genetically modified with a nucleic acid or a recombinant expression vector of the present disclosure.

In some cases, a nucleic acid of the present disclosure comprises a nucleotide sequence encoding: a) an HCV E1 polypeptide of an affinity tagged HCV E1/E2 heterodimer of the present disclosure; b) an HCV E1-affinity tag fusion polypeptide of an affinity tagged HCV E1/E2 heterodimer of the present disclosure; c) an HCV E2 polypeptide of an affinity tagged HCV E1/E2 heterodimer of the present disclosure; d) an HCV E2-affinity tag fusion polypeptide of an affinity tagged HCV E1/E2 heterodimer of the present disclosure; or e) an affinity tagged HCV E1/E2 heterodimer of the present disclosure (e.g., where both polypeptides of the heterodimer are encoded in a single nucleic acid). Thus, in some cases, a nucleic acid of the present disclosure comprises a nucleotide sequence encoding one polypeptide chain of an affinity tagged HCV E1/E2 heterodimer of the present disclosure.

In some cases, a nucleic acid of the present disclosure comprises a nucleotide sequence encoding: a) an HCV E1 polypeptide of an affinity tagged HCV E1/E2 heterodimer of the present disclosure; b) an HCV E1-Fc fusion polypeptide of an affinity tagged HCV E1/E2 heterodimer of the present disclosure; c) an HCV E2 polypeptide of an affinity tagged HCV E1/E2 heterodimer of the present disclosure; d) an HCV E2-Fc fusion polypeptide of an affinity tagged HCV E1/E2 heterodimer of the present disclosure; or e) an affinity tagged HCV E1/E2 heterodimer of the present disclosure (e.g., where both polypeptides of the heterodimer are encoded in a single nucleic acid). Thus, in some cases, a nucleic acid of the present disclosure comprises a nucleotide sequence encoding one polypeptide chain of an affinity tagged HCV E1/E2 heterodimer of the present disclosure.

Where a nucleic acid of the present disclosure comprises a nucleotide sequence encoding one polypeptide chain of an affinity tagged HCV E1/E2 heterodimer of the present disclosure, the nucleotide sequence can be operably linked to a promoter, e.g., a promoter that is functional in a eukaryotic cell. In other instances, a nucleic acid of the present disclosure comprises a nucleotide sequence encoding both polypeptide chains of an affinity tagged HCV E1/E2 heterodimer of the present disclosure; the nucleotide sequence can be operably linked to a promoter, e.g., a promoter that is functional in a eukaryotic cell.

The present disclosure provides a composition comprising: a) a first nucleic acid comprising a nucleotide sequence encoding an HCV E1 polypeptide; and b) a second nucleic acid comprising a nucleotide sequence encoding an HCV E2 polypeptide, where at least one of the HCV E1 and HCV E2 polypeptides is a fusion polypeptide comprising an affinity tag polypeptide. The present disclosure provides a composition comprising: a) a first nucleic acid comprising a nucleotide sequence encoding an HCV E1 polypeptide; and b) a second nucleic acid comprising a nucleotide sequence encoding an HCV E2 polypeptide, where at least one of the HCV E1 and HCV E2 polypeptides is a fusion polypeptide comprising an Ig Fc polypeptide.

Where a nucleic acid of the present disclosure comprises a nucleotide sequence encoding both polypeptide chains of an HCV E1/E2 heterodimer of the present disclosure, the nucleotide sequence can be operably linked to a promoter, e.g., a promoter that is functional in a eukaryotic cell. Where a nucleic acid of the present disclosure comprises a nucleotide sequence encoding both polypeptide chains of an HCV E1/E2 heterodimer of the present disclosure, the nucleic acid can comprise a first nucleotide sequence encoding an HCV E1 polypeptide, and a second nucleotide sequence encoding an HCV E2 polypeptide, where one or both of the E1 and E2 polypeptides is a fusion polypeptide comprising an Fc polypeptide. In some cases, the first nucleotide sequence and the second nucleotide sequence are separated by an internal ribosome entry site (IRES). In some cases, the first nucleotide sequence and the second nucleotide sequence are separated by a ribosomal skipping sequence (e.g., a nucleotide sequence encoding a 2A peptide (e.g., VKQTLNFDLLKLAGDVESNPGP (SEQ ID NO:41); see, e.g., Radcliffe and Mitrophanous (2004) Gene Therapy 11:1673).

In some cases, a nucleic acid of the present disclosure is inserted into an expression vector, to generate a recombinant expression vector. Thus, the present disclosure provides a recombinant expression vector comprising a nucleic acid comprising a nucleotide sequence encoding: a) an HCV E1 polypeptide of an affinity tagged HCV E1/E2 heterodimer of the present disclosure; b) an HCV E1-Fc fusion polypeptide of an affinity tagged HCV E1/E2 heterodimer of the present disclosure; c) an HCV E2 polypeptide of an affinity tagged HCV E1/E2 heterodimer of the present disclosure; d) an HCV E2-Fc fusion polypeptide of an affinity tagged HCV E1/E2 heterodimer of the present disclosure; or e) an affinity tagged HCV E1/E2 heterodimer of the present disclosure (e.g., where both polypeptides of the heterodimer are encoded in a single nucleic acid).

In some cases, the nucleotide sequence encoding a) an HCV E1 polypeptide of an affinity tagged HCV E1/E2 heterodimer of the present disclosure; b) an HCV E1-Fc fusion polypeptide of an affinity tagged HCV E1/E2 heterodimer of the present disclosure; c) an HCV E2 polypeptide of an affinity tagged HCV E1/E2 heterodimer of the present disclosure; d) an HCV E2-Fc fusion polypeptide of an affinity tagged HCV E1/E2 heterodimer of the present disclosure; or e) an affinity tagged HCV E1/E2 heterodimer of the present disclosure is operably linked to a transcriptional control element, e.g., a promoter, such as a promoter functional in a eukaryotic cell.

In some cases, a recombinant expression vector of the present disclosure comprises a nucleotide sequence encoding only one polypeptide chain of an affinity tagged HCV E1/E2 heterodimer of the present disclosure. The present disclosure provides a composition comprising: a) a first recombinant expression vector comprising a nucleotide sequence encoding an HCV E1 polypeptide; and b) a second recombinant expression vector comprising a nucleotide sequence encoding an HCV E2 polypeptide, where at least one of the HCV E1 and HCV E2 polypeptides is a fusion polypeptide comprising an Ig Fc polypeptide. The nucleotide sequence encoding the first polypeptide chain (HCV E1 polypeptide or HCV E1-Fc fusion polypeptide) or the second polypeptide chain (HCV E2 polypeptide or HCV E2-Fc fusion polypeptide) can be operably linked to a promoter, e.g., a promoter functional in a eukaryotic host cell.

In some cases, a recombinant expression vector of the present disclosure comprises a nucleotide sequence encoding both polypeptide chains of an affinity tagged HCV E1/E2 heterodimer of the present disclosure. In some instances, the nucleotide sequence is operably linked to a promoter, e.g., a promoter that is functional in a eukaryotic cell. Where a recombinant expression vector of the present disclosure comprises a nucleotide sequence encoding both polypeptide chains of an affinity tagged HCV E1/E2 heterodimer of the present disclosure, the recombinant expression vector can comprise a first nucleotide sequence encoding an HCV E1 polypeptide, and a second nucleotide sequence encoding an HCV E2 polypeptide, where one or both of the E1 and E2 polypeptides is a fusion polypeptide comprising an affinity tag (e.g., an Ig Fc polypeptide). In some cases, the first nucleotide sequence and the second nucleotide sequence are separated by an internal ribosome entry site (IRES). In some cases, the first nucleotide sequence and the second nucleotide sequence are separated by a ribosomal skipping sequence (e.g., a nucleotide sequence encoding a 2A peptide (e.g., VKQTLNFDLLKLAGDVESNPGP (SEQ ID NO:41); see, e.g., Radcliffe and Mitrophanous (2004) Gene Therapy 11:1673).

The present disclosure provide a nucleic acid comprising a nucleotide sequence encoding an affinity tagged HCV E1/E2 heterodimer of the present disclosure, where the affinity tagged HCV E1/E2 heterodimer comprises: i) an E1 polypeptide and an E2-affinity tag fusion polypeptide; ii) an E2-affinity tag fusion polypeptide and an E1 polypeptide; or iii) an E1-affinity tag fusion polypeptide and an E2-affinity tag fusion polypeptide. In some cases, a nucleotide sequence encoding a proteolytically cleavable linker is interposed between the nucleotide sequence encoding the HCV E1 or the HCV E2 polypeptide and the affinity tag.

The proteolytically cleavable linker can include a protease recognition sequence recognized by a protease selected from the group consisting of alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase, gastricsin, gelatinase, Gly-X carboxypeptidase, glycyl endopeptidase, human rhinovirus 3C protease, hypodermin C, IgA-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin, tissue kallikrein, tobacco etch virus (TEV), togavirin, tryptophanyl aminopeptidase, U-plasminogen activator, V8, venombin A, venombin AB, and Xaa-pro aminopeptidase.

For example, the proteolytically cleavable linker can comprise a matrix metalloproteinase (MMP) cleavage site, e.g., a cleavage site for a MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). For example, the cleavage sequence of MMP-9 is Pro-X-X-Hy (wherein, X represents an arbitrary residue; Hy, a hydrophobic residue), e.g., Pro-X-X-Hy-(Ser/Thr), e.g., Pro-Leu/Gln-Gly-Met-Thr-Ser (SEQ ID NO:17) or Pro-Leu/Gln-Gly-Met-Thr (SEQ ID NO:18). Another example of a protease cleavage site is a plasminogen activator cleavage site, e.g., a uPA or a tissue plasminogen activator (tPA) cleavage site. Another example of a suitable protease cleavage site is a prolactin cleavage site. Specific examples of cleavage sequences of uPA and tPA include sequences comprising Val-Gly-Arg. Another example of a protease cleavage site that can be included in a proteolytically cleavable linker is a tobacco etch virus (TEV) protease cleavage site, e.g., ENLYFQS (SEQ ID NO:2), where the protease cleaves between the glutamine and the serine. Another example of a protease cleavage site that can be included in a proteolytically cleavable linker is an enterokinase cleavage site, e.g., DDDDK (SEQ ID NO:3), where cleavage occurs after the lysine residue. Another example of a protease cleavage site that can be included in a proteolytically cleavable linker is a thrombin cleavage site, e.g., LVPR (SEQ ID NO:4) (e.g., where the proteolytically cleavable linker comprises the sequence LVPRGS (SEQ ID NO:5)). Additional suitable linkers comprising protease cleavage sites include linkers comprising one or more of the following amino acid sequences: LEVLFQGP (SEQ ID NO:1), cleaved by PreScission protease (a fusion protein comprising human rhinovirus 3C protease and glutathione-S-transferase; Walker et al. (1994) Biotechnol. 12:601); a thrombin cleavage site, e.g., CGLVPAGSGP (SEQ ID NO:19); SLLKSRMVPNFN (SEQ ID NO:20) or SLLIARRMPNEN (SEQ ID NO:21), cleaved by cathepsin B; SKLVQASASGVN (SEQ ID NO:22) or SSYLKASDAPDN (SEQ ID NO:23), cleaved by an Epstein-Barr virus protease; RPKPQQFFGLMN (SEQ ID NO:24) cleaved by MMP-3 (stromelysin); SLRPLALWRSFN (SEQ ID NO:25) cleaved by MMP-7 (matrilysin); SPQGIAGQRNFN (SEQ ID NO:26) cleaved by MMP-9; DVDERDVRGFASFL SEQ ID NO: 27) cleaved by a thermolysin-like MMP; SLPLGLWAPNFN (SEQ ID NO:28) cleaved by matrix metalloproteinase 2 (MMP-2); SLLIFRSWANFN (SEQ ID NO:29) cleaved by cathespin L; SGVVIATVIVIT (SEQ ID NO:30) cleaved by cathepsin D; SLGPQGIWGQFN (SEQ ID NO: 31) cleaved by matrix metalloproteinase 1 (MMP-1); KKSPGRVVGGSV (SEQ ID NO:32) cleaved by urokinase-type plasminogen activator; PQGLLGAPGILG (SEQ ID NO:33) cleaved by membrane type 1 matrixmetalloproteinase (MT-MMP); HGPEGLRVGFYESDVMGRGHARLVHVEEPHT (SEQ ID NO:34) cleaved by stromelysin 3 (or MMP-11), thermolysin, fibroblast collagenase and stromelysin-1; GPQGLAGQRGIV (SEQ ID NO: 35) cleaved by matrix metalloproteinase 13 (collagenase-3); GGSGQRGRKALE (SEQ ID NO: 36) cleaved by tissue-type plasminogen activator (tPA); SLSALLSSDIFN (SEQ ID NO: 37) cleaved by human prostate-specific antigen; SLPRFKIIGGFN (SEQ ID NO:38) cleaved by kallikrein (hK3); SLLGIAVPGNFN (SEQ ID NO:39) cleaved by neutrophil elastase; and FFKNIVTPRTPP (SEQ ID NO:40) cleaved by calpain (calcium activated neutral protease).

As noted above, a nucleic acid encoding one or both polypeptides of an affinity tagged HCV E1/E2 heterodimer of the present disclosure can be present in an expression vector. Suitable expression vectors include, but are not limited to, baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, herpes simplex virus, human immunodeficiency virus (HIV)-based lentivirus vectors, murine leukemia virus (MVL)-based gamma retrovirus vectors, and the like), P1-based artificial chromosomes, yeast plasmids, yeast artificial chromosomes, and any other vectors specific for specific hosts of interest (such as E. coli, mammalian cells, insect cells, or yeast cells). Suitable expression vectors include, but are not limited to, a replication-defective adenovirus vector; a replication-defective vaccinia virus vector; a lentivirus vector (e.g., a self-inactivating lentivirus vector); a retroviral vector (e.g., a self-inactivating retroviral vector); an adeno-associated virus vector; and the like. In some cases, the vector is a modified vaccinia Ankara (MVA) vector, or an MVA-based vector (see, e.g., Verheust et al. (2012) Vaccine 30:2623).

The present disclosure provides genetically modified host cells, where the genetically modified host cells are genetically modified with a nucleic acid(s) or recombinant expression vector(s) of the present disclosure.

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, MRC4 fibroblast cells, and the like.

Methods for introduction of nucleic acids into host cells include, for example, transformation, electroporation, conjugation, calcium phosphate methods and the like. The method for transfer can be selected so as to provide for stable expression of the introduced polypeptide-encoding nucleic acid. The polypeptide-encoding nucleic acid can be provided as an inheritable episomal element (e.g., a plasmid) or can be genomically integrated.

### 1) Nucleic Acid Encoding an HCV E1-Affinity Tag-E2 Polyprotein to Produce an Affinity Tagged HCV E1/E2 Heterodimer Comprising an HCV E2-Affinity Tag Fusion Polypeptide (N-Terminal Affinity Tagged E2) and an HCV E1 Polypeptide

In some cases, a nucleic acid of the present disclosure comprises a nucleotide sequence encoding a polyprotein comprising, in order from N-terminus to C-terminus: i) an HCV E1 polypeptide; and ii) an HCV E2-affinity tag fusion polypeptide, where the HCV E2-affinity tag fusion polypeptide comprises, in order from N-terminus to C-terminus: i) an affinity tag polypeptide; and ii) an HCV E2 polypeptide. Upon expression in a suitable host cell, an affinity tagged HCV E1/E2 heterodimer is produced, where the affinity tagged HCV E1/E2 heterodimer comprises an HCV E1 polypeptide and an HCV E2-affinity tag fusion polypeptide. The HCV E2-affinity tag fusion polypeptide so produced comprises, in order from N-terminus to C-terminus: i) an affinity tag polypeptide; and ii) an HCV E2 polypeptide.

In some cases, a nucleic acid of the present disclosure comprises a nucleotide sequence encoding a polyprotein comprising, in order from N-terminus to C-terminus: i) an HCV E1 polypeptide; and ii) an HCV E2-affinity tag fusion polypeptide, where the HCV E2-affinity tag fusion polypeptide comprises, in order from N-terminus to C-terminus: i) an affinity tag polypeptide; ii) a proteolytically cleavable linker; and iii) an HCV E2 polypeptide. Upon expression in a suitable host cell, an affinity tagged HCV E1/E2 heterodimer is produced, where the affinity tagged HCV E1/E2 heterodimer comprises an HCV E1 polypeptide and an HCV E2-affinity tag fusion polypeptide. The HCV E2-affinity tag fusion polypeptide so produced comprises, in order from N-terminus to C-terminus: i) an affinity tag polypeptide; ii) a proteolytically cleavable linker; and iii) an HCV E2 polypeptide.

In some cases, a nucleic acid of the present disclosure comprises a nucleotide sequence encoding a polyprotein comprising, in order from N-terminus to C-terminus: i) an HCV E1 polypeptide; ii) from 2 amino acids to 15 amino acids (aa) (e.g., 2 aa, 3 aa, 4 aa, 5 aa, 6 aa, 7 aa, 8 aa, 9 aa, 10 aa, 11 aa, 12 aa, 13 aa, 14 aa, or 15 aa) from the N-terminus of an HCV E2 polypeptide; iii) an affinity tag; iv) a proteolytically cleavable linker; and v) an HCV E2 polypeptide. In some cases, the nucleotide sequence encoding the polyprotein is operably linked to a transcription control element, e.g., a promoter that is functional in a eukaryotic cell. Suitable promoters include, e.g., a CMV promoter, an SV40 promoter, and the like, as described above. In some cases, the nucleic acid is present in an expression vector (e.g., a lentivirus vector; an adenoassociated virus vector; an adenovirus vector; a retroviral vector; a non-viral vector; etc.). In some cases, the affinity tag is an Ig Fc polypeptide. In other cases, the affinity tag is Protein A, Protein G, a polypeptide comprising a poly(histidine) tract, or a GST polypeptide. Suitable proteolytically cleavable linkers are described above. Upon expression in a suitable host cell, an affinity tagged HCV E1/E2 heterodimer is produced, where the affinity tagged HCV E1/E2 heterodimer comprises an HCV E1 polypeptide and an HCV E2-affinity tag fusion polypeptide. The HCV E2-affinity tag fusion polypeptide so produced comprises, in order from N-terminus to C-terminus: i) an affinity tag polypeptide; and an HCV E2 polypeptide.

In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) an HCV E1 polypeptide; ii) from 2 amino acids to 15 amino acids (aa) (e.g., 2 aa, 3 aa, 4 aa, 5 aa, 6 aa, 7 aa, 8 aa, 9 aa, 10 aa, 11 aa, 12 aa, 13 aa, 14 aa, or 15 aa) from the N-terminus of an HCV E2 polypeptide; iii) an affinity tag; iv) a proteolytically cleavable linker; and v) an HCV E2 polypeptide. In some cases, the polyprotein comprises a signal peptide. Thus, for example, in some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) a signal peptide; ii) an HCV E1 polypeptide; iii) from 2 amino acids to 15 amino acids (aa) (e.g., 2 aa, 3 aa, 4 aa, 5 aa, 6 aa, 7 aa, 8 aa, 9 aa, 10 aa, 11 aa, 12 aa, 13 aa, 14 aa, or 15 aa) from the N-terminus of an HCV E2 polypeptide; iv) an affinity tag; v) a proteolytically cleavable linker; and vi) an HCV E2 polypeptide. Suitable promoters include, e.g., a CMV promoter, an SV40 promoter, and the like. In some cases, the nucleic acid is present in an expression vector (e.g., a lentivirus vector; an adenoassociated virus vector; an adenovirus vector; a retroviral vector; a non-viral vector; etc.). In some cases, the affinity tag is an Ig Fc polypeptide. In other cases, the affinity tag is Protein A, Protein G, a polypeptide comprising a poly(histidine) tract, or a GST polypeptide. In some cases, the amino acids from the N-terminus of an HCV E2 polypeptide are a QT, ET, HT, GT, TT, RH, NT, AY, VI, or ST dipeptide.

Suitable signal peptides include, e.g., a tissue plasminogen activator (tPA) signal peptide; a prolactin signal peptide; an Ig kappa light chain precursor signal peptide; a serum albumin preproprotein signal peptide; an Immunoglobulin heavy chain signal peptide; an Immunoglobulin light chain signal peptide; an azuorcidin preproprotein signal peptide; a cystatin-S precursor signal peptide; a trypsinogen-2 precursor signal peptide; a chymotrypsinogen precursor signal peptide; and the like. (Bendtsen et al. (2004) J. Mol. Biol. 340 783-795; Kober et al. (2012) Biotechnology and Bioengineering 110 (4) 1164-1173). A suitable signal peptide includes a signalase cleavage site, such that a polyprotein comprising the signal peptide is cleaved, during intracellular processing, at the signalase cleavage site. In some cases, a suitable signal peptide comprises the amino acid sequence MDAMKRGLCCVLLLCGAVFVSPSQEIHARFRRGARS (SEQ ID NO:42). In some cases, a suitable signal peptide comprises the amino acid sequence

(SEQ ID NO: 6)
MDAMKRGLCCVLLLCGAVFVSPS.

In some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) an HCV E1 polypeptide; ii) from 2 amino acids to 15 amino acids (aa) (e.g., 2 aa, 3 aa, 4 aa, 5 aa, 6 aa, 7 aa, 8 aa, 9 aa, 10 aa, 11 aa, 12 aa, 13 aa, 14 aa, or 15 aa) from the N-terminus of an HCV E2 polypeptide; iii) an affinity tag; iv) a proteolytically cleavable linker; and v) an HCV E2 polypeptide. In some cases, the polyprotein comprises a signal peptide. Thus, for example, in some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) a signal peptide; ii) an HCV E1 polypeptide; iii) from 2 amino acids to 15 amino acids (aa) (e.g., 2 aa, 3 aa, 4 aa, 5 aa, 6 aa, 7 aa, 8 aa, 9 aa, 10 aa, 11 aa, 12 aa, 13 aa, 14 aa, or 15 aa) from the N-terminus of an HCV E2 polypeptide; iv) an affinity tag; v) a proteolytically cleavable linker; and vi) an HCV E2 polypeptide. In some cases, the nucleic acid is present in an expression vector (e.g., a lentivirus vector; an adenoassociated virus vector; an adenovirus vector; a retroviral vector; a non-viral vector; etc.). In some cases, the affinity tag is an Ig Fc polypeptide. In other cases, the affinity tag is Protein A, Protein G, a polypeptide comprising a poly(histidine) tract, or a GST polypeptide. In some cases, the amino acids from the N-terminus of an HCV E2 polypeptide are a QT, ET, HT, GT, TT, RH, NT, AY, VI, or ST dipeptide.

In some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) an HCV E1 polypeptide; ii) from 2 amino acids to 15 amino acids (aa) (e.g., 2 aa, 3 aa, 4 aa, 5 aa, 6 aa, 7 aa, 8 aa, 9 aa, 10 aa, 11 aa, 12 aa, 13 aa, 14 aa, or 15 aa) from the N-terminus of an HCV E2 polypeptide; iii) an Ig Fc polypeptide; iv) a proteolytically cleavable linker; and v) an HCV E2 polypeptide. In some cases, the polyprotein comprises a signal peptide. Thus, for example, in some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) a signal peptide; ii) an HCV E1 polypeptide; iii) from 2 amino acids to 15 amino acids (aa) (e.g., 2 aa, 3 aa, 4 aa, 5 aa, 6 aa, 7 aa, 8 aa, 9 aa, 10 aa, 11 aa, 12 aa, 13 aa, 14 aa, or 15 aa) from the N-terminus of an HCV E2 polypeptide; iv) an Ig Fc polypeptide; v) a proteolytically cleavable linker; and vi) an HCV E2 polypeptide. In some cases, the nucleic acid is present in an expression vector (e.g., a lentivirus vector; an adenoassociated virus vector; an adenovirus vector; a retroviral vector; a non-viral vector; etc.). In some cases, the amino acids from the N-terminus of an HCV E2 polypeptide are a QT, ET, HT, GT, TT, RH, NT, AY, VI, or ST dipeptide.

In some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) an HCV E1 polypeptide; ii) an ET (Glu-Thr) dipeptide from the N-terminus of an HCV E2 polypeptide; iii) an Ig Fc polypeptide; iv) a proteolytically cleavable linker; and v) an HCV E2 polypeptide. In some cases, the polyprotein comprises a signal peptide. Thus, for example, in some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) a signal peptide; ii) an HCV E1 polypeptide; iii) an ET (Glu-Thr) dipeptide from the N-terminus of an HCV E2 polypeptide; iv) an Ig Fc polypeptide; v) a proteolytically cleavable linker; and vi) an HCV E2 polypeptide. In some cases, the nucleic acid is present in an expression vector (e.g., a lentivirus vector; an adenoassociated virus vector; an adenovirus vector; a retroviral vector; a non-viral vector; etc.).

In some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) an HCV E1 polypeptide; ii) a GT (Gly-Thr) dipeptide from the N-terminus of an HCV E2 polypeptide; iii) an Ig Fc polypeptide; iv) a proteolytically cleavable linker; and v) an HCV E2 polypeptide. In some cases, the polyprotein comprises a signal peptide. Thus, for example, in some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) a signal peptide; ii) an HCV E1 polypeptide; iii) a GT (Gly-Thr) dipeptide from the N-terminus of an HCV E2 polypeptide; iv) an Ig Fc polypeptide; v) a proteolytically cleavable linker; and vi) an HCV E2 polypeptide. In some cases, the nucleic acid is present in an expression vector (e.g., a lentivirus vector; an adenoassociated virus vector; an adenovirus vector; a retroviral vector; a non-viral vector; etc.).

In some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) an HCV E1 polypeptide; ii) a TT (Thr-Thr) dipeptide from the N-terminus of an HCV E2 polypeptide; iii) an Ig Fc polypeptide; iv) a proteolytically cleavable linker; and v) an HCV E2 polypeptide. In some cases, the polyprotein comprises a signal peptide. Thus, for example, in some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) a signal peptide; ii) an HCV E1 polypeptide; iii) a TT (Thr-Thr) dipeptide from the N-terminus of an HCV E2 polypeptide; iv) an Ig Fc polypeptide; v) a proteolytically cleavable linker; and vi) an HCV E2 polypeptide. In some cases, the nucleic acid is present in an expression vector (e.g., a lentivirus vector; an adenoassociated virus vector; an adenovirus vector; a retroviral vector; a non-viral vector; etc.).

In some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) an HCV E1 polypeptide; ii) from 2 amino acids to 15 amino acids (aa) (e.g., 2 aa, 3 aa, 4 aa, 5 aa, 6 aa, 7 aa, 8 aa, 9 aa, 10 aa, 11 aa, 12 aa, 13 aa, 14 aa, or 15 aa) from the N-terminus of an HCV E2 polypeptide; iii) an Ig Fc polypeptide; iv) a proteolytically cleavable linker comprising the amino acid sequence LEVLFQGP (SEQ ID NO:1); and v) an HCV E2 polypeptide. In some cases, the polyprotein comprises a signal peptide. Thus, for example, in some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) a signal peptide; ii) an HCV E1 polypeptide; iii) from 2 amino acids to 15 amino acids (aa) (e.g., 2 aa, 3 aa, 4 aa, 5 aa, 6 aa, 7 aa, 8 aa, 9 aa, 10 aa, 11 aa, 12 aa, 13 aa, 14 aa, or 15 aa) from the N-terminus of an HCV E2 polypeptide; iv) an Ig Fc polypeptide; v) a proteolytically cleavable linker comprising the amino acid sequence LEVLFQGP (SEQ ID NO:1); and vi) an HCV E2 polypeptide. In some cases, the nucleic acid is present in an expression vector (e.g., a lentivirus vector; an adenoassociated virus vector; an adenovirus vector; a retroviral vector; a non-viral vector; etc.). In some cases, the amino acids from the N-terminus of an HCV E2 polypeptide are a QT, ET, HT, GT, TT, RH, NT, AY, VI, or ST dipeptide.

In some cases, a nucleic acid of the present disclosure comprises, in order from 5′ to 3′ and in operable linkage: a) a promoter; b) a nucleotide sequence encoding a polyprotein comprising, in order from N-terminus to C-terminus: i) a signal peptide having the amino acid sequence MDAMKRGLCCVLLLCGAVFVSPS (SEQ ID NO:6); ii) an HCV E1 polypeptide; iii) the dipeptide QT or ET; iv) an Ig Fc polypeptide; v) a proteolytically cleavable linker having the amino acid sequence LEVLFQGP (SEQ ID NO:1); and vi) an HCV E2 polypeptide. In some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) a signal peptide having the amino acid sequence MDAMKRGLCCVLLLCGAVFVSPS (SEQ ID NO:6); ii) an HCV E1 polypeptide; iii) the dipeptide QT or ET; iv) an Ig Fc polypeptide; v) a proteolytically cleavable linker having the amino acid sequence LEVLFQGP (SEQ ID NO:1); and vi) an HCV E2 polypeptide.

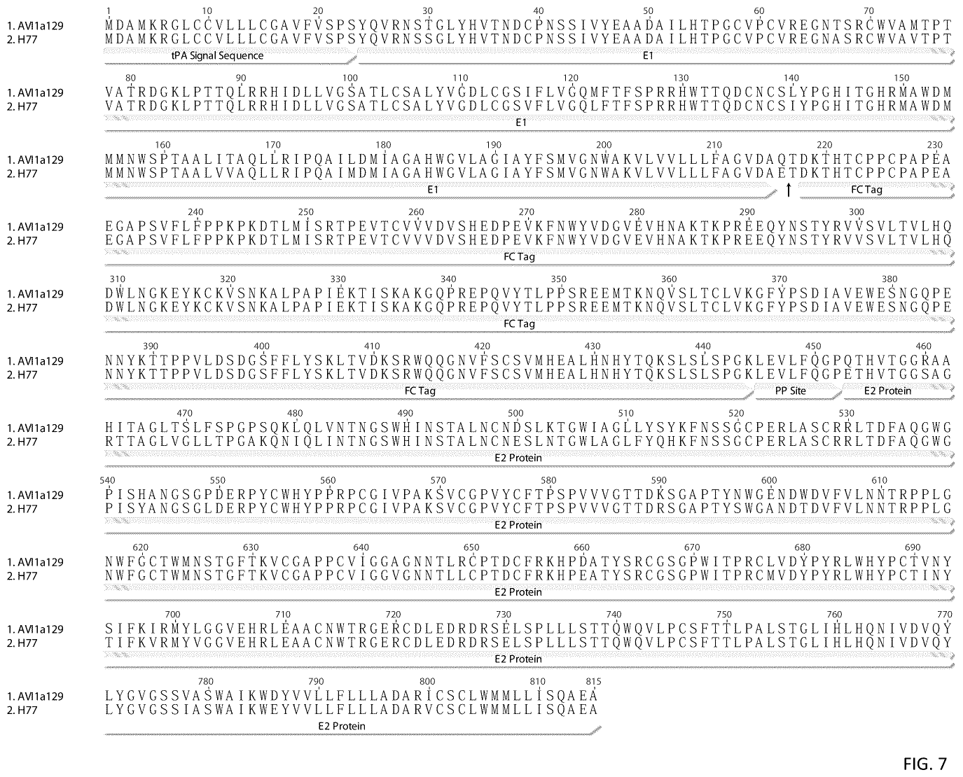

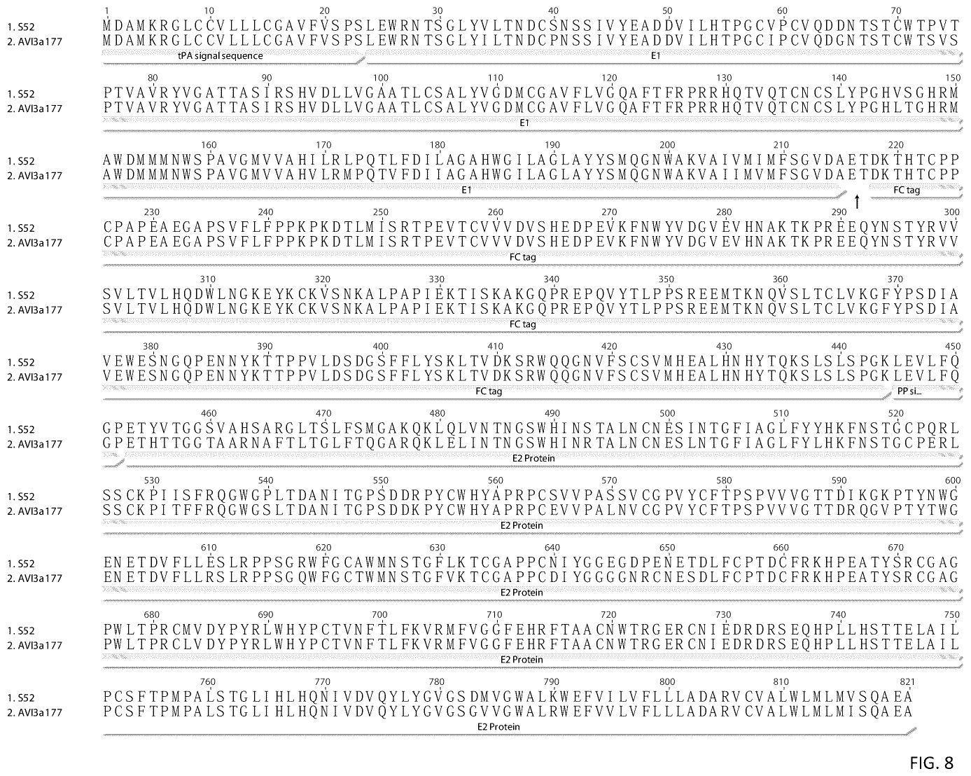

In some cases, a nucleic acid of the present disclosure comprises, in order from 5′ to 3′ and in operable linkage and in operable linkage, a) a promoter; and b) a nucleotide sequence encoding an amino acid sequence having at least 80%, at least 85%, at least 90%, at least 95%, at least 98%, at least 99%, or 100%, amino acid sequence identity to an amino acid sequence depicted in FIG. 7 or FIG. 8.

### 2) Nucleic Acid Encoding an HCV E1-E2-Affinity Tag Polyprotein to Produce an Affinity Tagged HCV E1/E2 Heterodimer Comprising an HCV E2-Affinity Tag Fusion Polypeptide (C-Terminal Affinity Tagged E2) and an HCV E1 Polypeptide

In some cases, a nucleic acid of the present disclosure comprises a nucleotide sequence encoding a polyprotein comprising, in order from N-terminus to C-terminus: i) an HCV E1 polypeptide; and ii) an HCV E2-affinity tag fusion polypeptide, where the HCV E2-affinity tag fusion polypeptide comprises, in order from N-terminus to C-terminus: i) an HCV E2 polypeptide; and ii) an affinity tag polypeptide. Upon expression in a suitable host cell, an affinity tagged HCV E1/E2 heterodimer is produced, where the affinity tagged HCV E1/E2 heterodimer comprises an HCV E1 polypeptide and an HCV E2-affinity tag fusion polypeptide. The HCV E2-affinity tag fusion polypeptide so produced comprises, in order from N-terminus to C-terminus: i) an HCV E2 polypeptide; and ii) an affinity tag polypeptide.

In some cases, a nucleic acid of the present disclosure comprises a nucleotide sequence encoding a polyprotein comprising, in order from N-terminus to C-terminus: i) an HCV E1 polypeptide; ii) an HCV E2 polypeptide; iii) a proteolytically cleavable linker; and iv) an affinity tag polypeptide.

In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) an HCV E1 polypeptide; ii) an HCV E2 polypeptide; iii) a proteolytically cleavable linker; and iv) an affinity tag polypeptide. In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) a signal peptide; ii) an HCV E1 polypeptide; iii) an HCV E2 polypeptide; iv) a proteolytically cleavable linker; and v) an affinity tag polypeptide.

Suitable signal peptides include, e.g., a tissue plasminogen activator (tPA) signal peptide; an Ig kappa light chain precursor signal peptide; a serum albumin preproprotein signal peptide; an Immunoglobulin heavy chain signal peptide; an Immunoglobulin light chain signal peptide; an azuorcidin preproprotein signal peptide; a cystatin-S precursor signal peptide; a trypsinogen-2 precursor signal peptide; a chymotrypsinogen precursor signal peptide; and the like. (Bendtsen et al. (2004) J. Mol. Biol. 340 783-795; Kober et al. (2012) Biotechnology and Bioengineering 110 (4) 1164-1173). A suitable signal peptide includes a signalase cleavage site, such that a polyprotein comprising the signal peptide is cleaved, during intracellular processing, at the signalase cleavage site. In some cases, a suitable signal peptide comprises the amino acid sequence MDAMKRGLCCVLLLCGAVFVSPSQEIHARFRRGARS (SEQ ID NO:42). In some cases, a suitable signal peptide comprises the amino acid sequence

(SEQ ID NO: 6)
MDAMKRGLCCVLLLCGAVFVSPS.

In some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) an HCV E1 polypeptide; ii) an HCV E2 polypeptide; iii) a proteolytically cleavable linker; and iv) an affinity tag polypeptide. In some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) a signal peptide; ii) an HCV E1 polypeptide; iii) an HCV E2 polypeptide; iv) a proteolytically cleavable linker; and v) an affinity tag polypeptide.

In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) an HCV E1 polypeptide; ii) an HCV E2 polypeptide; iii) a proteolytically cleavable linker; and iv) an Ig Fc polypeptide. In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) a signal peptide; ii) an HCV E1 polypeptide; iii) an HCV E2 polypeptide; iv) a proteolytically cleavable linker; and v) an Ig Fc polypeptide.

3) Nucleic acid encoding an HCV E1-affinity tag-E2 polyprotein to produce an affinity tagged HCV E1/E2 heterodimer comprising an HCV E1-affinity tag fusion polypeptide (N-terminal affinity tagged E1) and an HCV E2 polypeptide

In some cases, a nucleic acid of the present disclosure comprises a nucleotide sequence encoding a polyprotein comprising, in order from N-terminus to C-terminus: i) an HCV E1-affinity tag fusion polypeptide; and iii) an HCV E2 polypeptide, where the HCV E1-affinity tag fusion polypeptide comprises, in order from N-terminus to C-terminus: i) an affinity tag polypeptide; and ii) an HCV E1 polypeptide. Upon expression in a suitable host cell, an affinity tagged HCV E1/E2 heterodimer is produced, where the affinity tagged HCV E1/E2 heterodimer comprises an affinity tagged HCV E1 polypeptide and an HCV E2 polypeptide. The HCV E1-affinity tag fusion polypeptide so produced comprises, in order from N-terminus to C-terminus: i) an affinity tag polypeptide; and ii) an HCV E1 polypeptide.

In some cases, a nucleic acid of the present disclosure comprises a nucleotide sequence encoding a polyprotein comprising, in order from N-terminus to C-terminus: i) an affinity tag polypeptide; ii) a proteolytically cleavable linker; iii) an HCV E1 polypeptide; and iv) an HCV E2 polypeptide.

In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) an affinity tag polypeptide; ii) a proteolytically cleavable linker; iii) an HCV E1 polypeptide; and iv) an HCV E2 polypeptide. In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) a signal peptide; ii) an affinity tag polypeptide; iii) a proteolytically cleavable linker; iv) an HCV E1 polypeptide; and v) an HCV E2 polypeptide.

Suitable signal peptides include, e.g., a tissue plasminogen activator (tPA) signal peptide; an Ig kappa light chain precursor signal peptide; a serum albumin preproprotein signal peptide; an Immunoglobulin heavy chain signal peptide; an Immunoglobulin light chain signal peptide; an azuorcidin preproprotein signal peptide; a cystatin-S precursor signal peptide; a trypsinogen-2 precursor signal peptide; a chymotrypsinogen precursor signal peptide; and the like. (Bendtsen et al. (2004) J. Mol. Biol. 340 783-795; Kober et al. (2012) Biotechnology and Bioengineering 110 (4) 1164-1173). A suitable signal peptide includes a signalase cleavage site, such that a polyprotein comprising the signal peptide is cleaved, during intracellular processing, at the signalase cleavage site. In some cases, a suitable signal peptide comprises the amino acid sequence MDAMKRGLCCVLLLCGAVFVSPSQEIHARFRRGARS (SEQ ID NO:42). In some cases, a suitable signal peptide comprises the amino acid sequence

(SEQ ID NO: 6)
MDAMKRGLCCVLLLCGAVFVSPS.

In some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) an affinity tag polypeptide; ii) a proteolytically cleavable linker; iii) an HCV E1 polypeptide; and iv) an HCV E2 polypeptide. In some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) a signal peptide; ii) an affinity tag polypeptide; iii) a proteolytically cleavable linker; iv) an HCV E1 polypeptide; and v) an HCV E2 polypeptide.

In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) an Ig Fc polypeptide; ii) a proteolytically cleavable linker; iii) an HCV E1 polypeptide; and iv) an HCV E2 polypeptide. In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) a signal peptide; ii) an Ig Fc polypeptide; iii) a proteolytically cleavable linker; iv) an HCV E1 polypeptide; and v) an HCV E2 polypeptide.

### 4) Nucleic Acid Encoding an HCV E1-Affinity Tag-E2 Polyprotein to Produce an Affinity Tagged HCV E1/E2 Heterodimer Comprising an HCV E1-Affinity Tag Fusion Polypeptide (C-Terminal Affinity Tagged E1) and an HCV E2 Polypeptide

In some cases, a nucleic acid of the present disclosure comprises a nucleotide sequence encoding a polyprotein comprising, in order from N-terminus to C-terminus: i) an HCV E1-affinity tag fusion polypeptide; and iii) an HCV E2 polypeptide, where the HCV E1-affinity tag fusion polypeptide comprises, in order from N-terminus to C-terminus: i) an HCV E1 polypeptide; and ii) an affinity tag polypeptide. Upon expression in a suitable host cell, an affinity tagged HCV E1/E2 heterodimer is produced, where the affinity tagged HCV E1/E2 heterodimer comprises an affinity tagged HCV E1 polypeptide and an HCV E2 polypeptide. The HCV E1-affinity tag fusion polypeptide so produced comprises, in order from N-terminus to C-terminus: i) an HCV E1 polypeptide; and ii) an affinity tag polypeptide.

In some cases, a nucleic acid of the present disclosure comprises a nucleotide sequence encoding a polyprotein comprising, in order from N-terminus to C-terminus: i) an HCV E1 polypeptide; ii) a proteolytically cleavable linker; iii) an affinity tag polypeptide; and iv) an HCV E2 polypeptide. In some cases, a signal peptide comprising a signalase cleavage site is interposed between the affinity tag polypeptide and the HCV E2 polypeptide.

In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) an HCV E1 polypeptide; ii) a proteolytically cleavable linker; iii) an affinity tag polypeptide; and iv) an HCV E2 polypeptide. In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) a signal peptide; ii) an HCV E1 polypeptide; iii) a proteolytically cleavable linker; iii) an affinity tag polypeptide; and v) an HCV E2 polypeptide. In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) an HCV E1 polypeptide; ii) a proteolytically cleavable linker; iii) an affinity tag polypeptide; iv) a signal peptide comprising a signalase cleavage site; and v) an HCV E2 polypeptide. In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) a signal peptide; ii) an HCV E1 polypeptide; iii) a proteolytically cleavable linker; iv) an affinity tag polypeptide; v) a signal peptide comprising a signalase cleavage site; and vi) an HCV E2 polypeptide.

Suitable signal peptides include, e.g., a tissue plasminogen activator (tPA) signal peptide; an Ig kappa light chain precursor signal peptide; a serum albumin preproprotein signal peptide; an Immunoglobulin heavy chain signal peptide; an Immunoglobulin light chain signal peptide; an azuorcidin preproprotein signal peptide; a cystatin-S precursor signal peptide; a trypsinogen-2 precursor signal peptide; a chymotrypsinogen precursor signal peptide; and the like. (Bendtsen et al. (2004) J. Mol. Biol. 340 783-795; Kober et al. (2012) Biotechnology and Bioengineering 110 (4) 1164-1173). A suitable signal peptide includes a signalase cleavage site, such that a polyprotein comprising the signal peptide is cleaved, during intracellular processing, at the signalase cleavage site. In some cases, a suitable signal peptide comprises the amino acid sequence MDAMKRGLCCVLLLCGAVFVSPSQEIHARFRRGARS (SEQ ID NO:42). In some cases, a suitable signal peptide comprises the amino acid sequence

(SEQ ID NO: 6)
MDAMKRGLCCVLLLCGAVFVSPS.

In some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) an HCV E1 polypeptide; ii) a proteolytically cleavable linker; iii) an affinity tag polypeptide; and iv) an HCV E2 polypeptide. In some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) a signal peptide; ii) an HCV E1 polypeptide; iii) a proteolytically cleavable linker; iv) an affinity tag polypeptide; and v) an HCV E2 polypeptide.

In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) an HCV E1 polypeptide; ii) a proteolytically cleavable linker; iii) an Fc polypeptide; and iv) an HCV E2 polypeptide. In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) a signal peptide; ii) an HCV E1 polypeptide; iii) a proteolytically cleavable linker; iv) an Fc polypeptide; and v) an HCV E2 polypeptide.

Where the nucleotide sequence encodes two signal peptides, in some cases, the two signal peptides are two different signal peptides.

5) Nucleic Acid Encoding an HCV E1-Affinity Tag-HCV E2-Affinity Tag Polyprotein to Produce an Affinity Tagged HCV E1/E2 Heterodimer Comprising an HCV E1-Affinity Tag Fusion Polypeptide (N-Terminal Affinity Tagged E1) and an HCV E2-Affinity Tag Fusion Polypeptide (N-Terminal Affinity Tagged E2)

In some cases, a nucleic acid of the present disclosure comprises a nucleotide sequence encoding a polyprotein comprising, in order from N-terminus to C-terminus: i) an HCV E1-affinity tag fusion polypeptide; and iii) an HCV E2-affinity tag fusion polypeptide, where the HCV E1-affinity tag fusion polypeptide comprises, in order from N-terminus to C-terminus: i) an affinity tag polypeptide; and ii) an HCV E1 polypeptide, and where the HCV E2-affinity tag fusion polypeptide comprises, in order from N-terminus to C-terminus: i) an affinity tag polypeptide; and ii) an HCV E2 polypeptide. Upon expression in a suitable host cell, an affinity tagged HCV E1/E2 heterodimer is produced, where the affinity tagged HCV E1/E2 heterodimer comprises an affinity tagged HCV E2 polypeptide and an affinity tagged HCV E2 polypeptide. The HCV E1-affinity tag fusion polypeptide so produced comprises, in order from N-terminus to C-terminus: i) an affinity tag polypeptide; and ii) an HCV E1 polypeptide. The HCV E2-affinity tag fusion polypeptide so produced comprises, in order from N-terminus to C-terminus: i) an affinity tag polypeptide; and ii) an HCV E2 polypeptide.

In some cases, a nucleic acid of the present disclosure comprises a nucleotide sequence encoding a polyprotein comprising, in order from N-terminus to C-terminus: i) an affinity tag polypeptide; ii) a proteolytically cleavable linker; iii) an HCV E1 polypeptide; iv) from 2 amino acids to 15 amino acids (aa) (e.g., 2 aa, 3 aa, 4 aa, 5 aa, 6 aa, 7 aa, 8 aa, 9 aa, 10 aa, 11 aa, 12 aa, 13 aa, 14 aa, or 15 aa) from the N-terminus of an HCV E2 polypeptide; iv) an affinity tag; v) a proteolytically cleavable linker; and vi) an HCV E2 polypeptide. In some cases, the nucleotide sequence encoding the polyprotein is operably linked to a transcription control element, e.g., a promoter that is functional in a eukaryotic cell. Suitable promoters include, e.g., a CMV promoter, an SV40 promoter, and the like, as described above. In some cases, the nucleic acid is present in an expression vector (e.g., a lentivirus vector; an adenoassociated virus vector; an adenovirus vector; a retroviral vector; a non-viral vector; etc.). In some cases, the affinity tag is an Ig Fc polypeptide. In other cases, the affinity tag is Protein A, Protein G, a polypeptide comprising a poly(histidine) tract, or a GST polypeptide. Suitable proteolytically cleavable linkers are described above. Upon expression in a suitable host cell, an affinity tagged HCV E1/E2 heterodimer is produced, where the affinity tagged HCV E1/E2 heterodimer comprises an HCV E1-affinity tag fusion polypeptide and an HCV E2-affinity tag fusion polypeptide. The HCV E2-affinity tag fusion polypeptide so produced comprises, in order from N-terminus to C-terminus: i) an affinity tag polypeptide; and an HCV E2 polypeptide. The HCV E1-affinity tag fusion polypeptide so produced comprises, in order from N-terminus to C-terminus: i) an affinity tag polypeptide; and an HCV E1 polypeptide.

In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) an affinity tag polypeptide; ii) a proteolytically cleavable linker; iii) an HCV E1 polypeptide; iv) from 2 amino acids to 15 amino acids (aa) (e.g., 2 aa, 3 aa, 4 aa, 5 aa, 6 aa, 7 aa, 8 aa, 9 aa, 10 aa, 11 aa, 12 aa, 13 aa, 14 aa, or 15 aa) from the N-terminus of an HCV E2 polypeptide; v) an affinity tag polypeptide; vi) a proteolytically cleavable linker; and vii) an HCV E2 polypeptide. In some cases, the polyprotein comprises a signal peptide. Thus, for example, in some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) a signal peptide; ii) an affinity tag polypeptide; iii) a proteolytically cleavable linker; iv) an HCV E1 polypeptide; v) from 2 amino acids to 15 amino acids (aa) (e.g., 2 aa, 3 aa, 4 aa, 5 aa, 6 aa, 7 aa, 8 aa, 9 aa, 10 aa, 11 aa, 12 aa, 13 aa, 14 aa, or 15 aa) from the N-terminus of an HCV E2 polypeptide; vi) an affinity tag; vii) a proteolytically cleavable linker; and viii) an HCV E2 polypeptide. Suitable promoters include, e.g., a CMV promoter, an SV40 promoter, and the like. In some cases, the nucleic acid is present in an expression vector (e.g., a lentivirus vector; an adenoassociated virus vector; an adenovirus vector; a retroviral vector; a non-viral vector; etc.). In some cases, the affinity tag is an Ig Fc polypeptide. In other cases, the affinity tag is Protein A, Protein G, a polypeptide comprising a poly(histidine) tract, or a GST polypeptide. In some cases, the amino acids from the N-terminus of an HCV E2 polypeptide are a QT, ET, HT, GT, TT, RH, NT, AY, VI, or ST dipeptide.

Suitable signal peptides include, e.g., a tissue plasminogen activator (tPA) signal peptide; an Ig kappa light chain precursor signal peptide; a serum albumin preproprotein signal peptide; an Immunoglobulin heavy chain signal peptide; an Immunoglobulin light chain signal peptide; an azuorcidin preproprotein signal peptide; a cystatin-S precursor signal peptide; a trypsinogen-2 precursor signal peptide; a chymotrypsinogen precursor signal peptide; and the like. (Bendtsen et al. (2004) J. Mol. Biol. 340 783-795; Kober et al. (2012) Biotechnology and Bioengineering 110 (4) 1164-1173). A suitable signal peptide includes a signalase cleavage site, such that a polyprotein comprising the signal peptide is cleaved, during intracellular processing, at the signalase cleavage site. In some cases, a suitable signal peptide comprises the amino acid sequence MDAMKRGLCCVLLLCGAVFVSPSQEIHARFRRGARS (SEQ ID NO:42). In some cases, a suitable signal peptide comprises the amino acid sequence

(SEQ ID NO: 6)
MDAMKRGLCCVLLLCGAVFVSPS.

In some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) an affinity tag polypeptide; ii) a proteolytically cleavable linker; iii) an HCV E1 polypeptide; iv) from 2 amino acids to 15 amino acids (aa) (e.g., 2 aa, 3 aa, 4 aa, 5 aa, 6 aa, 7 aa, 8 aa, 9 aa, 10 aa, 11 aa, 12 aa, 13 aa, 14 aa, or 15 aa) from the N-terminus of an HCV E2 polypeptide; v) an affinity tag; vi) a proteolytically cleavable linker; and vii) an HCV E2 polypeptide. In some cases, the polyprotein comprises a signal peptide. Thus, for example, in some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) an affinity tag polypeptide; ii) a proteolytically cleavable linker; iii) an HCV E1 polypeptide; iv) from 2 amino acids to 15 amino acids (aa) (e.g., 2 aa, 3 aa, 4 aa, 5 aa, 6 aa, 7 aa, 8 aa, 9 aa, 10 aa, 11 aa, 12 aa, 13 aa, 14 aa, or 15 aa) from the N-terminus of an HCV E2 polypeptide; v) an affinity tag; vi) a proteolytically cleavable linker; and vii) an HCV E2 polypeptide. In some cases, the nucleic acid is present in an expression vector (e.g., a lentivirus vector; an adenoassociated virus vector; an adenovirus vector; a retroviral vector; a non-viral vector; etc.). In some cases, the affinity tag is an Ig Fc polypeptide. In other cases, the affinity tag is Protein A, Protein G, a polypeptide comprising a poly(histidine) tract, or a GST polypeptide. In some cases, the amino acids from the N-terminus of an HCV E2 polypeptide are a QT, ET, HT, GT, TT, RH, NT, AY, VI, or ST dipeptide.

In some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) an Ig Fc polypeptide; ii) a proteolytically cleavable linker; iii) an HCV E1 polypeptide; iv) from 2 amino acids to 15 amino acids (aa) (e.g., 2 aa, 3 aa, 4 aa, 5 aa, 6 aa, 7 aa, 8 aa, 9 aa, 10 aa, 11 aa, 12 aa, 13 aa, 14 aa, or 15 aa) from the N-terminus of an HCV E2 polypeptide; v) an Ig Fc polypeptide; vi) a proteolytically cleavable linker; and vii) an HCV E2 polypeptide. In some cases, the polyprotein comprises a signal peptide. Thus, for example, in some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) a signal peptide; ii) an Ig Fc polypeptide; iii) a proteolytically cleavable linker; iv) an HCV E1 polypeptide; v) from 2 amino acids to 15 amino acids (aa) (e.g., 2 aa, 3 aa, 4 aa, 5 aa, 6 aa, 7 aa, 8 aa, 9 aa, 10 aa, 11 aa, 12 aa, 13 aa, 14 aa, or 15 aa) from the N-terminus of an HCV E2 polypeptide; vi) an Ig Fc polypeptide; vii) a proteolytically cleavable linker; and viii) an HCV E2 polypeptide. In some cases, the nucleic acid is present in an expression vector (e.g., a lentivirus vector; an adenoassociated virus vector; an adenovirus vector; a retroviral vector; a non-viral vector; etc.). In some cases, the amino acids from the N-terminus of an HCV E2 polypeptide are a QT, ET, HT, GT, TT, RH, NT, AY, VI, or ST dipeptide.

In some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) an Ig Fc polypeptide; ii) a proteolytically cleavable linker; iii) an HCV E1 polypeptide; iv) an ET (Glu-Thr) dipeptide from the N-terminus of an HCV E2 polypeptide; v) an Ig Fc polypeptide; vi) a proteolytically cleavable linker; and vii) an HCV E2 polypeptide. In some cases, the polyprotein comprises a signal peptide. Thus, for example, in some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) a signal peptide; ii) an Ig Fc polypeptide; iii) a proteolytically cleavable linker; iv) an HCV E1 polypeptide; v) an ET (Glu-Thr) dipeptide from the N-terminus of an HCV E2 polypeptide; vi) an Ig Fc polypeptide; vii) a proteolytically cleavable linker; and viii) an HCV E2 polypeptide. In some cases, the nucleic acid is present in an expression vector (e.g., a lentivirus vector; an adenoassociated virus vector; an adenovirus vector; a retroviral vector; a non-viral vector; etc.).

In some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) an Ig Fc polypeptide; ii) a proteolytically cleavable linker; iii) an HCV E1 polypeptide; iv) a GT (Gly-Thr) dipeptide from the N-terminus of an HCV E2 polypeptide; v) an Ig Fc polypeptide; vi) a proteolytically cleavable linker; and vii) an HCV E2 polypeptide. In some cases, the polyprotein comprises a signal peptide. Thus, for example, in some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) a signal peptide; ii) an Ig Fc polypeptide; iii) a proteolytically cleavable linker; iv) an HCV E1 polypeptide; v) a GT (Gly-Thr) dipeptide from the N-terminus of an HCV E2 polypeptide; vi) an Ig Fc polypeptide; vi) a proteolytically cleavable linker; and viii) an HCV E2 polypeptide. In some cases, the nucleic acid is present in an expression vector (e.g., a lentivirus vector; an adenoassociated virus vector; an adenovirus vector; a retroviral vector; a non-viral vector; etc.).

In some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) an Ig Fc polypeptide; ii) a proteolytically cleavable linker; iii) an HCV E1 polypeptide; iv) a TT (Thr-Thr) dipeptide from the N-terminus of an HCV E2 polypeptide; v) an Ig Fc polypeptide; vi) a proteolytically cleavable linker; and vii) an HCV E2 polypeptide. In some cases, the polyprotein comprises a signal peptide. Thus, for example, in some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) a signal peptide; ii) an Ig Fc polypeptide; iii) a proteolytically cleavable linker; iv) an HCV E1 polypeptide; v) a TT (Thr-Thr) dipeptide from the N-terminus of an HCV E2 polypeptide; vi) an Ig Fc polypeptide; vii) a proteolytically cleavable linker; and viii) an HCV E2 polypeptide. In some cases, the nucleic acid is present in an expression vector (e.g., a lentivirus vector; an adenoassociated virus vector; an adenovirus vector; a retroviral vector; a non-viral vector; etc.).

In some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) an Ig Fc polypeptide; ii) a proteolytically cleavable linker; iii) an HCV E1 polypeptide; iv) from 2 amino acids to 15 amino acids (aa) (e.g., 2 aa, 3 aa, 4 aa, 5 aa, 6 aa, 7 aa, 8 aa, 9 aa, 10 aa, 11 aa, 12 aa, 13 aa, 14 aa, or 15 aa) from the N-terminus of an HCV E2 polypeptide; v) an Ig Fc polypeptide; vi) a proteolytically cleavable linker comprising the amino acid sequence LEVLFQGP (SEQ ID NO:1); and vii) an HCV E2 polypeptide. In some cases, the polyprotein comprises a signal peptide. Thus, for example, in some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) a signal peptide; ii) an Ig Fc polypeptide; iii) a proteolytically cleavable linker; iv) an HCV E1 polypeptide; v) from 2 amino acids to 15 amino acids (aa) (e.g., 2 aa, 3 aa, 4 aa, 5 aa, 6 aa, 7 aa, 8 aa, 9 aa, 10 aa, 11 aa, 12 aa, 13 aa, 14 aa, or 15 aa) from the N-terminus of an HCV E2 polypeptide; vi) an Ig Fc polypeptide; vi) a proteolytically cleavable linker comprising the amino acid sequence LEVLFQGP (SEQ ID NO: 1); and viii) an HCV E2 polypeptide. In some cases, the nucleic acid is present in an expression vector (e.g., a lentivirus vector; an adenoassociated virus vector; an adenovirus vector; a retroviral vector; a non-viral vector; etc.). In some cases, the amino acids from the N-terminus of an HCV E2 polypeptide are a QT, ET, HT, GT, TT, RH, NT, AY, VI, or ST dipeptide.

6) Nucleic Acid Encoding an HCV E1-Affinity Tag-HCV E2-Affinity Tag Polyprotein to Produce an Affinity Tagged HCV E1/E2 Heterodimer Comprising an HCV E1-Affinity Tag Fusion Polypeptide (C-Terminal Affinity Tagged E1) and an HCV E2-Affinity Tag Fusion Polypeptide (C-Terminal Affinity Tagged E2)

In some cases, a nucleic acid of the present disclosure comprises a nucleotide sequence encoding a polyprotein comprising, in order from N-terminus to C-terminus: i) an HCV E1-affinity tag fusion polypeptide; and iii) an HCV E2-affinity tag fusion polypeptide, where the HCV E1-affinity tag fusion polypeptide comprises, in order from N-terminus to C-terminus: i) an HCV E1 polypeptide; and ii) an affinity tag polypeptide; and where the HCV E2-affinity tag fusion polypeptide comprises, in order from N-terminus to C-terminus: i) an HCV E2 polypeptide; and ii) an affinity tag polypeptide. Upon expression in a suitable host cell, an affinity tagged HCV E1/E2 heterodimer is produced, where the affinity tagged HCV E1/E2 heterodimer comprises an affinity tagged HCV E2 polypeptide and an affinity tagged HCV E2 polypeptide. The HCV E1-affinity tag fusion polypeptide so produced comprises, in order from N-terminus to C-terminus: i) an HCV E1 polypeptide; and ii) an affinity tag polypeptide. The HCV E2-affinity tag fusion polypeptide so produced comprises, in order from N-terminus to C-terminus: i) an HCV E2 polypeptide; and ii) an affinity tag polypeptide.

In some cases, a nucleic acid of the present disclosure comprises a nucleotide sequence encoding a polyprotein comprising, in order from N-terminus to C-terminus: i) an HCV E1 polypeptide; ii) a proteolytically cleavable linker; iii) an affinity tag polypeptide; iv) an HCV E2 polypeptide; v) a proteolytically cleavable linker; and vi) an affinity tag polypeptide. In some cases, a signal peptide comprising a signalase cleavage site is interposed between the affinity tag polypeptide and the HCV E2 polypeptide.

In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) an HCV E1 polypeptide; ii) a proteolytically cleavable linker; iii) an affinity tag polypeptide; iv) an HCV E2 polypeptide; v) a proteolytically cleavable linker; and vi) an affinity tag polypeptide. In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) a signal peptide; ii) an HCV E1 polypeptide; iii) a proteolytically cleavable linker; iv) an affinity tag polypeptide; v) an HCV E2 polypeptide; vi) a proteolytically cleavable linker; and vii) an affinity tag polypeptide. In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) an HCV E1 polypeptide; ii) a proteolytically cleavable linker; iii) an affinity tag polypeptide; iv) a signal peptide comprising a signalase cleavage site; v) an HCV E2 polypeptide; vi) a proteolytically cleavable linker; and vii) an affinity tag polypeptide. In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) a signal peptide; ii) an HCV E1 polypeptide; iii) a proteolytically cleavable linker; iv) an affinity tag polypeptide; v) a signal peptide comprising a signalase cleavage site; vi) an HCV E2 polypeptide; vii) a proteolytically cleavable linker; and viii) an affinity tag polypeptide.

Suitable signal peptides include, e.g., a tissue plasminogen activator (tPA) signal peptide; an Ig kappa light chain precursor signal peptide; a serum albumin preproprotein signal peptide; an Immunoglobulin heavy chain signal peptide; an Immunoglobulin light chain signal peptide; an azuorcidin preproprotein signal peptide; a cystatin-S precursor signal peptide; a trypsinogen-2 precursor signal peptide; a chymotrypsinogen precursor signal peptide; and the like. (Bendtsen et al. (2004) J. Mol. Biol. 340 783-795; Kober et al. (2012) Biotechnology and Bioengineering 110 (4) 1164-1173). A suitable signal peptide includes a signalase cleavage site, such that a polyprotein comprising the signal peptide is cleaved, during intracellular processing, at the signalase cleavage site. In some cases, a suitable signal peptide comprises the amino acid sequence MDAMKRGLCCVLLLCGAVFVSPSQEIHARFRRGARS (SEQ ID NO:42). In some cases, a suitable signal peptide comprises the amino acid sequence

(SEQ ID NO: 6)
MDAMKRGLCCVLLLCGAVFVSPS.

In some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) an HCV E1 polypeptide; ii) a proteolytically cleavable linker; iii) an affinity tag polypeptide; iv) an HCV E2 polypeptide; v) a proteolytically cleavable linker; and vi) an affinity tag polypeptide. In some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) a signal peptide; ii) an HCV E1 polypeptide; iii) a proteolytically cleavable linker; iv) an affinity tag polypeptide; v) an HCV E2 polypeptide; vi) a proteolytically cleavable linker; and vii) an affinity tag polypeptide.

In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) an HCV E1 polypeptide; ii) a proteolytically cleavable linker; iii) an Fc polypeptide; and iv) an HCV E2 polypeptide. In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) a signal peptide; ii) an HCV E1 polypeptide; iii) a proteolytically cleavable linker; iv) an Fc polypeptide; v) an HCV E2 polypeptide; vi) a proteolytically cleavable linker; and vii) an affinity tag polypeptide.

Where the nucleotide sequence encodes two signal peptides, in some cases, the two signal peptides are two different signal peptides. Where the nucleotide sequence encodes two affinity tag polypeptides, in some cases, the two affinity tag polypeptides are the same. Where the nucleotide sequence encodes two affinity tag polypeptides, in some cases, the two affinity tag polypeptides are two different affinity tag polypeptides.

7) Nucleic Acid Encoding Encoding an HCV E1-Affinity Tag-HCV E2-Affinity Tag Polyprotein to Produce an Affinity Tagged HCV E1/E2 Heterodimer Comprising an HCV E1-Affinity Tag Fusion Polypeptide (N-Terminal Affinity Tagged E1) and an HCV E2-Affinity Tag Fusion Polypeptide (C-Terminal Affinity Tagged E2)

In some cases, a nucleic acid of the present disclosure comprises a nucleotide sequence encoding a polyprotein comprising, in order from N-terminus to C-terminus: i) an HCV E1-affinity tag fusion polypeptide; and ii) an HCV E2-affinity tag fusion polypeptide, where the HCV E1-affinity tag fusion polypeptide comprises, in order from N-terminus to C-terminus: i) an affinity tag polypeptide; and ii) an HCV E1 polypeptide; and where the HCV E2-affinity tag fusion polypeptide comprises, in order from N-terminus to C-terminus: i) an HCV E2 polypeptide; and ii) an affinity tag polypeptide. Upon expression in a suitable host cell, an affinity tagged HCV E1/E2 heterodimer is produced, where the affinity tagged HCV E1/E2 heterodimer comprises an affinity tagged HCV E2 polypeptide and an affinity tagged HCV E2 polypeptide. The HCV E1-affinity tag fusion polypeptide so produced comprises, in order from N-terminus to C-terminus: i) an affinity tag polypeptide; and ii) an HCV E1 polypeptide. The HCV E2-affinity tag fusion polypeptide so produced comprises, in order from N-terminus to C-terminus: i) an HCV E2 polypeptide; and ii) an affinity tag polypeptide.

In some cases, a nucleic acid of the present disclosure comprises a nucleotide sequence encoding a polyprotein comprising, in order from N-terminus to C-terminus: i) an affinity tag polypeptide; ii) a proteolytically cleavable linker; iii) an HCV E1 polypeptide; iv) an HCV E2 polypeptide; v) a proteolytically cleavable linker; and vi) an affinity tag polypeptide.

In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) an affinity tag polypeptide; ii) a proteolytically cleavable linker; iii) an HCV E1 polypeptide; iv) an HCV E2 polypeptide; v) a proteolytically cleavable linker; and vi) an affinity tag polypeptide. In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) a signal peptide; ii) an affinity tag polypeptide; iii) a proteolytically cleavable linker; iv) an HCV E1 polypeptide; v) an HCV E2 polypeptide; vi) a proteolytically cleavable linker; and vii) an affinity tag polypeptide. In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) an affinity tag polypeptide; ii) a proteolytically cleavable linker; iii) an HCV E1 polypeptide; iv) an HCV E2 polypeptide; v) a proteolytically cleavable linker; and vi) an affinity tag polypeptide. In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) a signal peptide;

ii) an affinity tag polypeptide; iii) a proteolytically cleavable linker; iv) an HCV E1 polypeptide; v) an HCV E2 polypeptide; vii) a proteolytically cleavable linker; and viii) an affinity tag polypeptide.

Suitable signal peptides include, e.g., a tissue plasminogen activator (tPA) signal peptide; an Ig kappa light chain precursor signal peptide; a serum albumin preproprotein signal peptide; an Immunoglobulin heavy chain signal peptide; an Immunoglobulin light chain signal peptide; an azuorcidin preproprotein signal peptide; a cystatin-S precursor signal peptide; a trypsinogen-2 precursor signal peptide; a chymotrypsinogen precursor signal peptide; and the like. (Bendtsen et al. (2004) J. Mol. Biol. 340 783-795; Kober et al. (2012) Biotechnology and Bioengineering 110 (4) 1164-1173). A suitable signal peptide includes a signalase cleavage site, such that a polyprotein comprising the signal peptide is cleaved, during intracellular processing, at the signalase cleavage site. In some cases, a suitable signal peptide comprises the amino acid sequence MDAMKRGLCCVLLLCGAVFVSPSQEIHARFRRGARS (SEQ ID NO:42). In some cases, a suitable signal peptide comprises the amino acid sequence

(SEQ ID NO: 6)
MDAMKRGLCCVLLLCGAVFVSPS.

In some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) an affinity tag polypeptide; ii) a proteolytically cleavable linker; iii) an HCV E1 polypeptide; iv) an HCV E2 polypeptide; v) a proteolytically cleavable linker; and vi) an affinity tag polypeptide. In some cases, a nucleic acid of the present disclosure comprises, in order from 5′ to 3′ and in operable linkage: a) a CMV promoter; b) a nucleotide sequence encoding a polyprotein comprising, in order from N-terminus to C-terminus: i) a signal peptide; ii) an affinity tag polypeptide; iii) a proteolytically cleavable linker; iv) an HCV E1 polypeptide; v) an HCV E2 polypeptide; vi) a proteolytically cleavable linker; and vii) an affinity tag polypeptide.

In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) an Fc polypeptide; ii) a proteolytically cleavable linker; iii) an HCV E1 polypeptide; and iv) an HCV E2 polypeptide. In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) a signal peptide; ii) an Fc polypeptide; iii) a proteolytically cleavable linker; iv) an HCV E1 polypeptide; v) an HCV E2 polypeptide; vi) a proteolytically cleavable linker; and vii) an affinity tag polypeptide.

8) Nucleic Acid Encoding an HCV E1-Affinity Tag-HCV E2-Affinity Tag Polyprotein to Produce an Affinity Tagged HCV E1/E2 Heterodimer Comprising an HCV E1-Affinity Tag Fusion Polypeptide (C-Terminal Affinity Tagged E1) and an HCV E2-Affinity Tag Fusion Polypeptide (N-Terminal Affinity Tagged E2)

In some cases, a nucleic acid of the present disclosure comprises a nucleotide sequence encoding a polyprotein comprising, in order from N-terminus to C-terminus: i) an HCV E1-affinity tag fusion polypeptide; and ii) an HCV E2-affinity tag fusion polypeptide, where the HCV E1-affinity tag fusion polypeptide comprises, in order from N-terminus to C-terminus: i) an HCV E1 polypeptide; and ii) an affinity tag polypeptide; and where the HCV E2-affinity tag fusion polypeptide comprises, in order from N-terminus to C-terminus: i) an affinity tag polypeptide; and ii) an HCV E2 polypeptide. Upon expression in a suitable host cell, an affinity tagged HCV E1/E2 heterodimer is produced, where the affinity tagged HCV E1/E2 heterodimer comprises an affinity tagged HCV E2 polypeptide and an affinity tagged HCV E2 polypeptide. The HCV E1-affinity tag fusion polypeptide so produced comprises, in order from N-terminus to C-terminus: i) an HCV E1 polypeptide; and ii) an affinity tag polypeptide. The HCV E2-affinity tag fusion polypeptide so produced comprises, in order from N-terminus to C-terminus: i) an affinity tag polypeptide; and ii) an HCV E2 polypeptide.

In some cases, a nucleic acid of the present disclosure comprises a nucleotide sequence encoding a polyprotein comprising, in order from N-terminus to C-terminus: i) an HCV E1 polypeptide; ii) a proteolytically cleavable linker; iii) an affinity tag polypeptide; iv) an affinity tag polypeptide; v) a proteolytically cleavable linker; and vi) an HCV E2 polypeptide.

In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) an HCV E1 polypeptide; ii) a proteolytically cleavable linker; iii) an affinity tag polypeptide; iv) an affinity tag polypeptide; v) a proteolytically cleavable linker; and vi) an HCV E2 polypeptide. In some cases, a nucleotide sequence encoding a signal peptide is interposed between the first and the second affinity tag polypeptides.

In some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) a first signal peptide; ii) an HCV E1 polypeptide; iii) a proteolytically cleavable linker; iv) a first affinity tag polypeptide; v) a second signal peptide; vi) a second affinity tag polypeptide; vii) a proteolytically cleavable linker; and viii) an HCV E2 polypeptide. In some cases, the first and the second affinity tag polypeptides are two different affinity tag polypeptides. In some cases, the first and the second signal peptides are two different signal peptides.

Suitable signal peptides include, e.g., a tissue plasminogen activator (tPA) signal peptide; an Ig kappa light chain precursor signal peptide; a serum albumin preproprotein signal peptide; an Immunoglobulin heavy chain signal peptide; an Immunoglobulin light chain signal peptide; an azuorcidin preproprotein signal peptide; a cystatin-S precursor signal peptide; a trypsinogen-2 precursor signal peptide; a chymotrypsinogen precursor signal peptide; and the like. (Bendtsen et al. (2004) J. Mol. Biol. 340 783-795; Kober et al. (2012) Biotechnology and Bioengineering 110 (4) 1164-1173). A suitable signal peptide includes a signalase cleavage site, such that a polyprotein comprising the signal peptide is cleaved, during intracellular processing, at the signalase cleavage site. In some cases, a suitable signal peptide comprises the amino acid sequence MDAMKRGLCCVLLLCGAVFVSPSQEIHARFRRGARS (SEQ ID NO:42). In some cases, a suitable signal peptide comprises the amino acid sequence

(SEQ ID NO: 6)
MDAMKRGLCCVLLLCGAVFVSPS.

### Methods of Producing an HCV E1/E2 Heterodimer

The present disclosure provides methods of producing an affinity tagged HCV E1/E2 heterodimer of the present disclosure. The two polypeptide chains of an affinity tagged HCV E1/E2 heterodimer of the present disclosure can be produced using any suitable method, e.g., recombinant and non-recombinant methods (e.g., chemical synthesis). Where the two polypeptide chains of an affinity tagged HCV E1/E2 heterodimer of the present disclosure are produced by recombinant methods, the two polypeptide chains can be produced in separate host cells, or in the same host cell. In some cases, the two polypeptide chains of an affinity tagged HCV E1/E2 heterodimer of the present disclosure is produced in the same host cell.

The present disclosure provides methods for producing an HCV E1/E2 heterodimer that is not affinity tagged. The methods generally involve producing an affinity tagged HCV E1/E2 heterodimer of the present disclosure; immobilizing the affinity tagged HCV E1/E2 heterodimer of the present disclosure on an immobilized binding partner for the affinity tag; and cleaving a linker between the affinity tag and the HCV E1 or E2 polypeptide, thereby releasing the untagged HCV E1/E2 heterodimer; and collecting the untagged HCV E1/E2 heterodimer.

Where the two polypeptide chains of an affinity tagged HCV E1/E2 heterodimer of the present disclosure are produced using recombinant techniques, the polypeptides may be produced as an intracellular protein or as an secreted protein, using any suitable construct and any suitable host cell, which can be a prokaryotic cell (e.g., a bacterial (e.g., Escherichia coli) cell), or a eukaryotic cell. Suitable eukaryotic cells include, e.g., a yeast host cell, an insect cell, a mammalian cell, filamentous fungi, and plant cells. Suitable yeast cells include, e.g., Saccharomyces cerevisiae and Pichia (e.g., Pichia pastoris).

Suitable mammalian cells include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, MRC5 cells (ATCC No. CCL-171), and the like. Where mammalian host cells are used, such host cells may include human cells (e.g., HeLa, 293, H9 and Jurkat cells); mouse cells (e.g., NIH3T3, L cells, and C127 cells); primate cells (e.g., Cos 1, Cos 7 and CV1); MRC4 cells; and hamster cells (e.g., Chinese hamster ovary (CHO) cells).

A variety of host-vector systems suitable for the expression of a polypeptide may be employed according to standard procedures known in the art. See, e.g., Sambrook et al., 1989 Current Protocols in Molecular Biology Cold Spring Harbor Press, New York; Ausubel et al. 1995 Current Protocols in Molecular Biology, Eds. Wiley and Sons; “Protein Expression: A Practical Approach” (1999) S. J. Higgins and B. D. James, eds., Oxford University Press; “Protein Expression in Mammalian Cells: Methods and Protocols (Methods in Molecular Biology)” (2012) James L. Hartley, ed., Humana Press; and “Production of Recombinant Proteins” (2005) Gerd Gellisen, ed., Wiley-VCH. Methods for introduction of nucleic acids into host cells include, for example, transformation, electroporation, conjugation, calcium phosphate methods and the like. The method for transfer can be selected so as to provide for stable expression of the introduced polypeptide-encoding nucleic acid. The polypeptide-encoding nucleic acid can be provided as an inheritable episomal element (e.g., a plasmid) or can be genomically-integrated. A variety of appropriate vectors for use in production of a peptide of interest are available commercially.

Suitable expression vectors include, but are not limited to, baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, herpes simplex virus, human immunodeficiency virus (HIV)-based lentivirus vectors, murine leukemia virus (MVL)-based gamma retrovirus vectors, and the like), P1-based artificial chromosomes, yeast plasmids, yeast artificial chromosomes, and any other vectors specific for specific hosts of interest (such as E. coli, mammalian cells, insect cells, or yeast cells).

An affinity tagged HCV E1/E2 heterodimer of the present disclosure can be produced by introducing a recombinant expression vector comprising a nucleotide sequence encoding the appropriate HCV E1 and E2 polypeptides (e.g., an E1 polypeptide and an E2-affinity tag fusion polypeptide; an E2-affinity tag fusion polypeptide and an E1 polypeptide; an E1-affinity tag fusion polypeptide and an E2-affinity tag fusion polypeptide) into an appropriate host cell, where the host cell produces the encoded polypeptides. In the expression vector, a polynucleotide comprising a nucleotide sequence(s) encoding the appropriate HCV E1 and E2 polypeptides (e.g., an E1 polypeptide and an E2-affinity tag fusion polypeptide; an E2-affinity tag fusion polypeptide and an E1 polypeptide; an E1-affinity tag fusion polypeptide and an E2-affinity tag fusion polypeptide) is linked to a regulatory sequence as appropriate to obtain the desired expression properties. These regulatory sequences can include promoters, enhancers, terminators, operators, repressors, and inducers. The promoters can be regulated or constitutive. Expression vectors generally have convenient restriction sites located near the promoter sequence to provide for the insertion of nucleic acid sequences encoding a protein of interest. A selectable marker operative in the expression host cell may be present.

In some cases, the polypeptides of an affinity tagged HCV E1/E2 heterodimer of the present disclosure (e.g., an E1 polypeptide and an E2-affinity tag fusion polypeptide; an E2-affinity tag fusion polypeptide and an E1 polypeptide; an E1-affinity tag fusion polypeptide and an E2-affinity tag fusion polypeptide) are encoded in a recombinant expression vector suitable for expression in a eukaryotic host cell (e.g., an insect cell; a yeast cell; a mammalian host cell, such as CHO cells, HeLa cells, 293 cells, MRC5 cells, etc.). In some cases, a recombinant expression vector comprises a nucleotide sequence encoding E1 and E2 polypeptides (one or both of which is a fusion polypeptide that includes an affinity tag polypeptide) as a single polypeptide chain; the recombinant expression vector is introduced into a eukaryotic host cell to generate a genetically modified host cell. In some cases, the two polypeptide chains of an HCV E1/E2 heterodimer of the present disclosure are initially produced as a single polypeptide chains, either in the same host cell or in separate host cells. Where the polypeptides of an affinity tagged HCV E1/E2 heterodimer of the present disclosure (e.g., an E1 polypeptide and an E2-affinity tag fusion polypeptide; an E2-affinity tag fusion polypeptide and an E1 polypeptide; an E1-affinity tag fusion polypeptide and an E2-affinity tag fusion polypeptide) are produced in the same host cell, the separate E1 and E2 polypeptides (one or both of which comprise an affinity tag) can form a heterodimer (e.g., a non-covalently linked heterodimer) in the endoplasmic reticulum (ER). In some cases, two polypeptide chains of an affinity tagged HCV E1/E2 heterodimer of the present disclosure are initially produced as a single polypeptide chain, which is cleaved in the ER of the genetically modified host cell to produce separate E1 and E2 polypeptides (one or both of which comprises an affinity tag). The separate E1 and E2 polypeptides (e.g., E1+E2-affinity tag; E2-affinity tag+E1; E1-affinity tag+E2-affinity tag) can form a heterodimer (e.g., a non-covalently linked heterodimer) in the ER. The affinity tagged HCV E1/E2 heterodimer can be isolated from the genetically modified host cell by, e.g., lysis using a non-ionic detergent, or using a freeze-thaw method. See, e.g., Frey et al. (2010) Vaccine 28:6367. The E1/E2 heterodimer can be purified from a cell lysate and/or cell culture medium using any of a variety of methods, including size exclusion chromatography, affinity chromatography, and the like, or combinations of such methods. In some cases, the affinity tagged HCV E1/E2 heterodimer is purified from cell lysate and/or cell culture medium using Galanthus nivalis (GNA) lectin affinity chromatography. In some cases, where the affinity tag is an Ig Fc, the affinity tagged HCV E1/E2 heterodimer is purified on an affinity column comprising an immobilized Fc-binding moiety. In some cases, where the affinity tag is a Protein A or Protein G polypeptide, the affinity tagged HCV E1/E2 heterodimer is purified on an affinity column comprising an immobilized Protein A-or Protein G-binding moiety. In some cases, where the affinity tag is an Ig light chain-containing polypeptide, the affinity tagged HCV E1/E2 heterodimer is purified on an affinity column comprising an immobilized Protein L polypeptide. In some cases, where the affinity tag is a GST polypeptide, the affinity tagged HCV E1/E2 heterodimer is purified on an affinity column comprising an immobilized GST-binding moiety. In some cases, the affinity tagged HCV E1/E2 heterodimer is purified from a cell lysate. In some cases, the affinity tagged HCV E1/E2 heterodimer is secreted from a cell and is purified from the cell culture medium. Suitable methods that can be used for purifying an affinity tagged HCV E1/E2 heterodimer of the present disclosure are described in, e.g., U.S. Pat. Nos. 6,121,020; 6,274,148; and Mazzocca et al. (2005) J. Biol. Chem. 280:11329. For example, in some cases, an affinity tagged HCV E1/E2 heterodimer can be prepared in a method comprising cell disruption and debris removal by microfiltration, followed by purification using three subsequent chromatographic steps: lectin affinity chromatography, hydroxyapatite chromatography, and ion exchange chromatography.

After production in a host cell(s), the two polypeptide chains of an affinity tagged HCV E1/E2 heterodimer of the present disclosure (e.g., as separate polypeptides or as a heterodimer) can be purified from the host cell(s). Methods of purification of recombinantly produced polypeptides from a host cell are known in the art and include, e.g., detergent lysis (e.g., with a non-ionic detergent) or freeze-thaw lysis, followed by one or more of size exclusion column chromatography, high performance liquid chromatography, affinity chromatography, and the like.

### Producing an Untagged HCV E1/E2 Heterodimer

In some cases, a method of producing an untagged HCV E1/E2 heterodimer disclosure comprises: a) contacting a lysate of a genetically modified host cell (where the genetically modified host cell produces an affinity tagged HCV E1/E2 heterodimer of the present disclosure (e.g., an HCV E2 polypeptide and an HCV E1-affinity tag fusion polypeptide; an HCV E2-affinity tag fusion polypeptide and an HCV E1 polypeptide; or an HCV E1-affinity tag fusion polypeptide and an HCV E2-affinity tag fusion polypeptide) with an affinity tag-binding polypeptide immobilized on an insoluble support, where the affinity tagged HCV E1/E2 heterodimer present in the lysate binds to the immobilized affinity tag-binding polypeptide, generating an immobilized affinity tagged HCV E1/E2 heterodimer; and b) contacting the immobilized affinity tagged HCV E1/E2 heterodimer with an enzyme that cleaves between the affinity tag and the affinity-tagged HCV E1 and/or E2 polypeptides, thereby releasing an untagged HCV E1/E2 heterodimer; and collecting the released untagged HCV E1/E2 heterodimer. In some cases, the untagged HCV E1/E2 heterodimer is subjected to one or more further purification steps.

Where the affinity tag is an Ig Fc polypeptide, suitable Ig Fc binding moieties include, but are not limited to, Protein A (Graille et al. (2000) Proc. Natl. Acad. Sci. USA 97:5399); Protein G (Sjöbring et al. (1991) J. Biol. Chem. 266:399); and a Protein A/G fusion polypeptide (Eliasson et al. (1988) J. Biol. Chem. 263:4323).

The Ig Fc binding moiety can be immobilized onto a solid support, where the solid support can be of any of a variety of forms, e.g., a bead, a magnetic bead, a plate, and the like. The solid support can be made of any of a variety of materials, including, but not limited to, polystyrene, agarose, polyesters, polyethylene, and the like.

An untagged HCV E1/E2 heterodimer can be purified such that the untagged HCV E1/E2 heterodimer is at least 60% pure, at least 65% pure, at least 70% pure, at least 75% pure, at least 80% pure, at least 85% pure, at least 90% pure, at least 95% pure, at least 98% pure, at least 99% pure, or greater than 99% pure.

### HCV E1/E2 Heterodimers

The present disclosure provides HCV E1/E2 heterodimers produced using a method of the present disclosure. In some cases, an HCV E1/E2 heterodimer of the present disclosure comprises a modified HCV E2 polypeptide with from 1 to 6 amino acids from the proteolytically cleavable linker on the N-terminus of the E2 polypeptide. In some cases, an HCV E1/E2 heterodimer of the present disclosure comprises a modified HCV E2 polypeptide with from 1 to 6 amino acids from the proteolytically cleavable linker on the C-terminus of the E2 polypeptide. In some cases, an HCV E1/E2 heterodimer of the present disclosure comprises a modified HCV E1 polypeptide with from 1 to 6 amino acids from the proteolytically cleavable linker on the N-terminus of the E1 polypeptide. In some cases, an HCV E1/E2 heterodimer of the present disclosure comprises a modified HCV E1 polypeptide with from 1 to 6 amino acids from the proteolytically cleavable linker on the C-terminus of the E1 polypeptide. In some cases, an HCV E1/E2 heterodimer of the present disclosure comprises a modified HCV E2 polypeptide and a modified HCV E1 polypeptide.

E2 with N-Terminal Heterologous Amino Acids

In some cases, an HCV E1/E2 heterodimer of the present disclosure comprises a modified HCV E2 polypeptide with from 1 to 6 amino acids from the proteolytically cleavable linker on the N-terminus of the E2 polypeptide. In some cases, a heterodimeric polypeptide of the present disclosure comprises: a) an HCV E1 polypeptide; and b) a modified E2 polypeptide comprising, in order from N-terminus to C-terminus: i) from 1 to 6 heterologous amino acids wherein the from 1 to 6 heterologous amino acids are C-terminal to a site of proteolytic cleavage in a proteolytically cleavable linker; and ii) an HCV E2 polypeptide.

Proteolytically cleavable linkers are described above. Following proteolytic cleavage of a precursor polypeptide, as described above, a modified E2 polypeptide is generated, which modified E2 polypeptide comprises, at its N-terminus, amino acids C-terminal to the protease cleavage site within the proteolytically cleavable linker.

For example, where the proteolytically cleavable linker comprises a PreScission cleavage site (LEVLFQGP; SEQ ID NO:1), where cleavage occurs between the glutamine and the glycine, a modified E2 polypeptide present in a heterodimeric polypeptide of the present disclosure comprises, in order from N-terminus to C-terminus: a) Gly-Pro; and b) an HCV E2 polypeptide.

As another example, where the proteolytically cleavable linker comprises a TEV cleavage site (ENLYFQS; SEQ ID NO:2), where cleavage occurs between the glutamine and the serine, a modified E2 polypeptide present in a heterodimeric polypeptide of the present disclosure comprises, in order from N-terminus to C-terminus: a) Ser; and b) an HCV E2 polypeptide.

As another example, where the proteolytically cleavable linker comprises a TEV cleavage site (ENLYFQG; SEQ ID NO:43), where cleavage occurs between the glutamine and the glycine, a modified E2 polypeptide present in a heterodimeric polypeptide of the present disclosure comprises, in order from N-terminus to C-terminus: a) Gly; and b) an HCV E2 polypeptide.

As another example, where the proteolytically cleavable linker comprises a thrombin cleavage site (LVPRGS; SEQ ID NO:5), where cleavage occurs between the arginine and the glycine, a modified E2 polypeptide present in a heterodimeric polypeptide of the present disclosure comprises, in order from N-terminus to C-terminus: a) Gly-Ser; and an HCV E2 polypeptide.

As another example, where the proteolytically cleavable linker comprises a Factor Xa cleavage site (I(E/D)GRX, where X is any amino acid except arginine or proline; SEQ ID NO: 44), where cleavage occurs between the arginine and the X, a modified E2 polypeptide present in a heterodimeric polypeptide of the present disclosure comprises, in order from N-terminus to C-terminus: a) X (where X is any amino acid except arginine or proline); and an HCV E2 polypeptide.

E1 with N-Terminal Heterologous Amino Acids

In some cases, an HCV E1/E2 heterodimer of the present disclosure comprises a modified HCV E1 polypeptide with from 1 to 6 amino acids from a proteolytically cleavable linker on the N-terminus of the E1 polypeptide. In some cases, a heterodimeric polypeptide of the present disclosure comprises: a) an HCV E2 polypeptide; and b) a modified E1 polypeptide comprising, in order from N-terminus to C-terminus: i) from 1 to 6 heterologous amino acids wherein the from 1 to 6 heterologous amino acids are C-terminal to a site of proteolytic cleavage in a proteolytically cleavable linker; and ii) an HCV E1 polypeptide.

Proteolytically cleavable linkers are described above. Following proteolytic cleavage of a precursor polypeptide (e.g., a precursor polypeptide comprising, in order from N-terminus to C-terminus: a) an Fc polypeptide or an HCV E2 polypeptide; b) a proteolytically cleavable linker; and c) an HCV E1 polypeptide), a modified E1 polypeptide is generated, which modified E1 polypeptide comprises, at its N-terminus, amino acids C-terminal to the protease cleavage site within the proteolytically cleavable linker.

For example, where the proteolytically cleavable linker comprises a PreScission cleavage site (LEVLFQGP; SEQ ID NO:1), where cleavage occurs between the glutamine and the glycine, a modified E1 polypeptide present in a heterodimeric polypeptide of the present disclosure comprises, in order from N-terminus to C-terminus: a) Gly-Pro; and b) an HCV E1 polypeptide.

As another example, where the proteolytically cleavable linker comprises a TEV cleavage site (ENLYFQS; SEQ ID NO:2), where cleavage occurs between the glutamine and the serine, a modified E1 polypeptide present in a heterodimeric polypeptide of the present disclosure comprises, in order from N-terminus to C-terminus: a) Ser; and b) an HCV E1 polypeptide.

As another example, where the proteolytically cleavable linker comprises a TEV cleavage site (ENLYFQG; SEQ ID NO:43), where cleavage occurs between the glutamine and the glycine, a modified E1 polypeptide present in a heterodimeric polypeptide of the present disclosure comprises, in order from N-terminus to C-terminus: a) Gly; and b) an HCV E1 polypeptide.

As another example, where the proteolytically cleavable linker comprises a thrombin cleavage site (LVPRGS; SEQ ID NO:5), where cleavage occurs between the arginine and the glycine, a modified E1 polypeptide present in a heterodimeric polypeptide of the present disclosure comprises, in order from N-terminus to C-terminus: a) Gly-Ser; and an HCV E1 polypeptide.

As another example, where the proteolytically cleavable linker comprises a Factor Xa cleavage site (I(E/D)GRX, where X is any amino acid except arginine or proline; SEQ ID NO: 44), where cleavage occurs between the arginine and the X, a modified E1 polypeptide present in a heterodimeric polypeptide of the present disclosure comprises, in order from N-terminus to C-terminus: a) X (where X is any amino acid except arginine or proline); and an HCV E1 polypeptide.

E2 with C-Terminal Heterologous Amino Acids

In some cases, an HCV E1/E2 heterodimer of the present disclosure comprises a modified HCV E2 polypeptide with from 1 to 6 amino acids from a proteolytically cleavable linker on the C-terminus of the E2 polypeptide. In some cases, a heterodimeric polypeptide of the present disclosure comprises: a) an HCV E1 polypeptide; and b) a modified E2 polypeptide comprising, in order from N-terminus to C-terminus: i) an HCV E2 polypeptide; and ii) from 1 to 6 heterologous amino acids wherein the from 1 to 6 heterologous amino acids are N-terminal to a site of proteolytic cleavage in a proteolytically cleavable linker.

Proteolytically cleavable linkers are described above. Following proteolytic cleavage of a precursor polypeptide (e.g., a precursor polypeptide comprising, in order from N-terminus to C-terminus: a) HCV E2 polypeptide; b) a proteolytically cleavable linker; and c) an Fc polypeptide or an HCV E1 polypeptide), a modified E2 polypeptide is generated, which modified E2 polypeptide comprises, at its C-terminus, amino acids N-terminal to the protease cleavage site within the proteolytically cleavable linker.

For example, where the proteolytically cleavable linker comprises a PreScission cleavage site (LEVLFQGP; SEQ ID NO:1), where cleavage occurs between the glutamine and the glycine, a modified E2 polypeptide present in a heterodimeric polypeptide of the present disclosure comprises, in order from N-terminus to C-terminus: a) an HCV E2 polypeptide; and b) LEVLFQ (SEQ ID NO:7).

As another example, where the proteolytically cleavable linker comprises an enterokinase cleavage site (DDDDK; SEQ ID NO:3), where cleavage occurs C-terminal to the Lys, a modified E2 polypeptide present in a heterodimeric polypeptide of the present disclosure comprises, in order from N-terminus to C-terminus: a) an HCV E2 polypeptide; and b) DDDDK (SEQ ID NO:3).

As another example, where the proteolytically cleavable linker comprises a TEV cleavage site (ENLYFQG; SEQ ID NO:43), where cleavage occurs between the glutamine and the glycine, a modified E2 polypeptide present in a heterodimeric polypeptide of the present disclosure comprises, in order from N-terminus to C-terminus: a) an HCV E2 polypeptide; and b) ENLYFQ (SEQ ID NO:8).

As another example, where the proteolytically cleavable linker comprises a thrombin cleavage site (LVPRGS; SEQ ID NO:5), where cleavage occurs between the arginine and the glycine, a modified E2 polypeptide present in a heterodimeric polypeptide of the present disclosure comprises, in order from N-terminus to C-terminus: a) an HCV E2 polypeptide; and LVPR (SEQ ID NO:4).

As another example, where the proteolytically cleavable linker comprises a Factor Xa cleavage site (I(E/D)GRX, where X is any amino acid except arginine or proline; SEQ ID NO: 44), where cleavage occurs between the arginine and the X, a modified E2 polypeptide present in a heterodimeric polypeptide of the present disclosure comprises, in order from N-terminus to C-terminus: a) an HCV E2 polypeptide; and I(E/D)GR (SEQ ID NO:45).

E1 with C-Terminal Heterologous Amino Acids

In some cases, an HCV E1/E2 heterodimer of the present disclosure comprises a modified HCV E1 polypeptide with from 1 to 6 amino acids from a proteolytically cleavable linker on the C-terminus of the E1 polypeptide. In some cases, a heterodimeric polypeptide of the present disclosure comprises: a) an HCV E2 polypeptide; and b) a modified E1 polypeptide comprising, in order from N-terminus to C-terminus: i) an HCV E1 polypeptide; and ii) from 1 to 6 heterologous amino acids wherein the from 1 to 6 heterologous amino acids are N-terminal to a site of proteolytic cleavage in a proteolytically cleavable linker.

Proteolytically cleavable linkers are described above. Following proteolytic cleavage of a precursor polypeptide (e.g., a precursor polypeptide comprising, in order from N-terminus to C-terminus: a) HCV E1 polypeptide; b) a proteolytically cleavable linker; and c) an Fc polypeptide or an HCV E2 polypeptide), a modified E1 polypeptide is generated, which modified E1 polypeptide comprises, at its C-terminus, amino acids N-terminal to the protease cleavage site within the proteolytically cleavable linker.

For example, where the proteolytically cleavable linker comprises a PreScission cleavage site (LEVLFQGP; SEQ ID NO:1), where cleavage occurs between the glutamine and the glycine, a modified E1 polypeptide present in a heterodimeric polypeptide of the present disclosure comprises, in order from N-terminus to C-terminus: a) an HCV E1 polypeptide; and b) LEVLFQ (SEQ ID NO:7).

As another example, where the proteolytically cleavable linker comprises an enterokinase cleavage site (DDDDK; SEQ ID NO:3), where cleavage occurs C-terminal to the Lys, a modified E1 polypeptide present in a heterodimeric polypeptide of the present disclosure comprises, in order from N-terminus to C-terminus: a) an HCV E1 polypeptide; and b) DDDDK (SEQ ID NO:3).

As another example, where the proteolytically cleavable linker comprises a TEV cleavage site (ENLYFQG; SEQ ID NO:43), where cleavage occurs between the glutamine and the glycine, a modified E1 polypeptide present in a heterodimeric polypeptide of the present disclosure comprises, in order from N-terminus to C-terminus: a) an HCV E1 polypeptide; and b) ENLYFQ (SEQ ID NO:8).

As another example, where the proteolytically cleavable linker comprises a thrombin cleavage site (LVPRGS; SEQ ID NO:5), where cleavage occurs between the arginine and the glycine, a modified E1 polypeptide present in a heterodimeric polypeptide of the present disclosure comprises, in order from N-terminus to C-terminus: a) an HCV E1 polypeptide; and LVPR (SEQ ID NO:4).

As another example, where the proteolytically cleavable linker comprises a Factor Xa cleavage site (I(E/D)GRX, where X is any amino acid except arginine or proline; SEQ ID NO: 44), where cleavage occurs between the arginine and the X, a modified E1 polypeptide present in a heterodimeric polypeptide of the present disclosure comprises, in order from N-terminus to C-terminus: a) an HCV E1 polypeptide; and I(E/D)GR (SEQ ID NO:45).

### Pharmaceutical Formulations

An HCV E1/E2 heterodimeric polypeptide of the present disclosure can be formulated with a pharmaceutically acceptable excipient(s) to generate an immunogenic composition. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7th ed., Lippincott, Williams, & Wilkins; and Handbook of Pharmaceutical Excipients (2000) A. H. Kibbe et al., eds., 3rd ed. Amer. Pharmaceutical Assoc.

In some cases, a heterodimeric HCV E1/E2 polypeptide of the present disclosure is formulated in an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some cases, the aqueous buffer further includes a non-ionic surfactant such as polysorbate 20 (TWEEN®20) or polysorbate 80 (TWEEN®80). For example, a formulation of E1 and variant E2 polypeptides in an aqueous buffer can include, e.g., from about 0.01% to about 0.05% polysorbate-20 (TWEEN®20) non-ionic detergent. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures. In some cases, the aqueous buffer further includes a non-ionic surfactant. In some cases, the aqueous buffer includes the non-ionic surfactant Triton™X-100, e.g., 0.1% Triton™X-100.

An heterodimeric HCV E1/E2 polypeptide of the present disclosure can be formulated into a preparation for injection by dissolving, suspending or emulsifying the heterodimer in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

An immunogenic composition of the present disclosure can include, e.g., pharmaceutical grades of mannitol, lactose, starch, magnesium stearate, sodium saccharin, talcum, cellulose, glucose, sucrose, magnesium, carbonate, and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like.

The concentration of a heterodimeric HCV E1/E2 polypeptide of the present disclosure in a formulation can vary widely (e.g., from less than about 0.1% to at least about 2%, to as much as 20% to 50% or more by weight) and can be selected primarily based on fluid volumes, viscosities, and patient-based factors in accordance with the particular mode of administration selected and the patient's needs.

The HCV E1/E2 heterodimeric polypeptide-containing formulations of the present disclosure can be provided in the form of a solution, suspension, tablet, pill, capsule, powder, gel, cream, lotion, ointment, aerosol or the like. It is recognized that oral administration can require protection of the compositions from digestion. This is typically accomplished either by association of the composition with an agent that renders it resistant to acidic and enzymatic hydrolysis or by packaging the composition in an appropriately resistant carrier. Means of protecting from digestion are well known in the art.

The HCV E1/E2 heterodimeric polypeptide polypeptide-containing formulations of the present disclosure can also be provided so as to enhance serum half-life of the heterodimer following administration. For example, where a heterodimeric HCV E1/E2 polypeptide of the present disclosure is formulated for injection, the heterodimeric HCV E1/E2 polypeptide may be provided in a liposome formulation, prepared as a colloid, or other conventional techniques for extending serum half-life. A variety of methods are available for preparing liposomes, as described in, e.g., Szoka et al., Ann. Rev. Biophys. Bioeng. 9:467 (1980), U.S. Pat. Nos. 4,235,871, 4,501,728 and 4,837,028. The preparations may also be provided in controlled release or slow-release forms.

### Adjuvant

An immunogenic composition of the present disclosure can include, in addition to a heterodimeric HCV E1/E2 polypeptide of the present disclosure, an adjuvant. Examples of known suitable adjuvants that can be used in humans include, but are not necessarily limited to, alum, aluminum phosphate, aluminum hydroxide, MF59 (4.3% w/v squalene, 0.5% w/vTWEEN 80TM (Polysorbate 80, a nonionic surfactant), 0.5% w/v Span 85), CpG-containing nucleic acid (where the cytosine is unmethylated), QS21, MPL, 3DMPL, extracts from Aquilla, ISCOMS, LT/CT mutants, poly(D,L-lactide-co-glycolide) (PLG) microparticles, Quil A, interleukins, and the like. For experimental animals, one can use Freund's, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dip-almitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/TWEEN 80 emulsion. Another suitable adjuvant is AS01, a suspension of liposomes comprising 3′-O-desacyl-4′-monophosphoryl lipid A (MPL) and Quillaja saponaria 21 (QS21). In some cases, the adjuvant is selected from MF59, AS01, AS02, AS03, AS04, alum, aluminum hydroxide, and aluminum phosphate. The effectiveness of an adjuvant may be determined by one or more of measuring the amount of antibodies directed against the immunogenic antigen or antigenic epitope thereof, measuring a cytotoxic T lymphocyte response to the antigen, and measuring a helper T cell response to the antigen.

Further exemplary adjuvants to enhance effectiveness of the composition include, but are not limited to: (1) oil-in-water emulsion formulations (with or without other specific immunostimulating agents such as muramyl peptides (see below) or bacterial cell wall components), such as for example (a) MF59TM (see, e.g., WO 90/14837), containing 5% Squalene, 0.5% TWEEN 80, and 0.5% Span 85 (optionally containing MTP-PE) formulated into submicron particles using a microfluidizer, (b) SAF, containing 10% Squalane, 0.4% TWEEN 80, 5% pluronic-blocked polymer L121, and thr-MDP either microfluidized into a submicron emulsion or vortexed to generate a larger particle size emulsion, and (c) RIBI™ adjuvant system (RAS), (Ribi Immunochem, Hamilton, Mont.) containing 2% Squalene, 0.2% TWEEN 80, and one or more bacterial cell wall components such as monophosphorylipid A (MPL), trehalose dimycolate (TDM), and cell wall skeleton (CWS), e.g., MPL+CWS (Detox™); (2) saponin adjuvants, such as QS21 or STIMULON™ (Cambridge Bioscience, Worcester, Mass.) may be used or particles generated therefrom such as ISCOMs (immunostimulating complexes), which ISCOMS may be devoid of additional detergent e.g. WO 00/07621; (3) Complete Freund's Adjuvant (CFA) and Incomplete Freund's Adjuvant (IFA); (4) cytokines, such as interleukins (e.g. IL-1, IL-2, IL-4, IL-5, IL-6, IL-7, IL-12 (WO99/44636), etc.), interferons (e.g. gamma interferon), macrophage colony stimulating factor (M-CSF), tumor necrosis factor (TNF), etc.; (5) monophosphoryl lipid A (MPL) or 3-O-deacylated MPL (3dMPL) e.g. GB-2220221, EP-A-0689454, optionally in the substantial absence of alum when used with pneumococcal saccharides e.g. WO 00/56358; (6) combinations of 3dMPL with, for example, QS21 and/or oil-in-water emulsions (see, e.g. EP-A-0835318, EP-A-0735898, EP-A-0761231); (7) oligonucleotides comprising a CpG motif containing at least one CG dinucleotide, where the cytosine is unmethylated (see, e.g., WO 96/02555, WO 98/16247, WO 98/18810, WO 98/40100, WO 98/55495, WO 98/37919 and WO 98/52581); (8) a polyoxyethylene ether or a polyoxyethylene ester (see, e.g. WO 99/52549); (9) a polyoxyethylene sorbitan ester surfactant in combination with an octoxynol (WO 01/21207) or a polyoxyethylene alkyl ether or ester surfactant in combination with at least one additional non-ionic surfactant such as an octoxynol (WO 01/21152); (10) a saponin and an immunostimulatory oligonucleotide (e.g. a CpG oligonucleotide) (WO 00/62800); (11) an immunostimulant and a particle of metal salt (see, e.g. WO 00/23105); (12) a saponin and an oil-in-water emulsion (see e.g. WO 99/11241); (13) a saponin (e.g. QS21)+3dMPL+IM2 (optionally including a sterol) (see, e.g. WO 98/57659); (14) other substances that act as immunostimulating agents to enhance the efficacy of the composition. Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipalmitoyl-sn-glycero-3-hydroxyphosphoryloxy)-ethylamine MTP-PE), etc. Also suitable for use is MATRIX™; MATRIX™ is an adjuvant that comprises 40 nm nanoparticles comprising Quillaja saponins, cholesterol, and phospholipid. Adjuvants suitable for administration to a human are of particular interest. In some cases, the adjuvant is one that enhances a CD4+ T helper response to the immunogen.

In some instances, the adjuvant is MF59, with or without a CpG-containing oligonucleotide. In other instances, the adjuvant is alum, with or without a CpG-containing oligonucleotide. In other instances, the adjuvant is poly(D,L-lactide-co-glycolide), with or without a CpG-containing oligonucleotide. In other instances, the adjuvant is MPL, with or without a CpG-containing oligonucleotide. In some cases, the adjuvant is Matrix-M™, with or without a CpG-containing oligonucleotide. In some cases, the adjuvant is keyhole limpet hemocyanin. In some instances, the adjuvant is AS01, a suspension of liposomes comprising 3′-O-desacyl-4′-monophosphoryl lipid A (MPL) and Quillaja saponaria 21 (QS21). In some cases, the adjuvant is MPL+alum.

### Methods of Inducing an Immune Response to HCV

The present disclosure provides a method of inducing an immune response (e.g., a protective immune response) to at least one HCV genotype in a mammalian subject. In some cases, the methods comprise administering to an individual in need thereof an effective amount of a heterodimeric HCV E1/E2 polypeptide of the present disclosure, or a composition (e.g., an immunogenic composition) comprising a heterodimeric HCV E1/E2 polypeptide of the present disclosure. In other cases, the methods comprise administering to an individual in need thereof an effective amount of a nucleic acid(s) (e.g., a recombinant expression vector) comprising nucleotide sequences encoding a heterodimeric polypeptide of the present disclosure.

An HCV immunogenic composition of the present disclosure, or a nucleic acid(s) comprising nucleotide sequences encoding a heterodimeric polypeptide of the present disclosure, is generally administered to a human subject who has an HCV infection or who is at risk of acquiring an HCV infection (e.g., is at greater risk than the general population of acquiring an HCV infection) so as to prevent or at least partially arrest the development of disease and its complications. An amount adequate to accomplish this is defined as a “therapeutically effective dose” or a “therapeutically effective amount.” “Prophylactic” use of a subject immunogenic composition generally refers to administration to an individual who has not been infected with HCV. “Therapeutic” use of a subject immunogenic composition can refer to “prophylactic” use (administration to an individual who has not been infected with HCV) and/or to administration to an individual who has an HCV infection. A “therapeutically effective amount” of an immunogenic composition of the present disclosure, can be an amount that, when administered in one or more doses to an individual who is not infected with HCV, is effective to induce an immune response in the individual to HCV. A “therapeutically effective amount” of an immunogenic composition of the present disclosure, can be an amount that, when administered in one or more doses to an individual who is infected with HCV, is effective to enhance an immune response in the individual to HCV.

Amounts effective for therapeutic use will depend on, e.g., the immunogenic composition or the nucleic acid(s) comprising nucleotide sequences encoding a heterodimeric polypeptide of the present disclosure, the manner of administration, the weight and general state of health of the patient, and the judgment of the prescribing physician. Single or multiple doses of a subject immunogenic composition, or a nucleic acid(s) comprising nucleotide sequences encoding a heterodimeric polypeptide of the present disclosure, can be administered depending on the dosage and frequency required and tolerated by the patient, and route of administration.

In some cases, an effective amount (e.g., a therapeutically effective amount) of an HCV E1/E2 immunogenic composition of the present disclosure, or a nucleic acid(s) comprising nucleotide sequences encoding a heterodimeric polypeptide of the present disclosure, is an amount that, when administered to an individual in one or more doses, is effective to induce an antibody response (e.g., a neutralizing antibody response) to HCV in the individual. For example, antibody to HCV (e.g., extracellular HCV), and/or to an HCV-infected cell, can be induced.

An effective amount of an HCV E1/E2 immunogenic composition of the present disclosure, or a nucleic acid(s) comprising nucleotide sequences encoding a heterodimeric polypeptide of the present disclosure, can be an amount that, when administered to an individual in one or more doses, is effective to induce a neutralizing antibody response to HCV of a variety of genotypes (e.g., genotype 1; genotype 3; etc.). A neutralizing antibody response reduces binding of HCV to one or more host receptors for HCV and inhibits entry of HCV into a cell.

In some cases, an effective amount (e.g., a therapeutically effective amount) of an HCV E1/E2 immunogenic composition of the present disclosure, or a nucleic acid(s) comprising nucleotide sequences encoding a heterodimeric polypeptide of the present disclosure, is an amount that, when administered to an individual in one or more doses, is effective to induce a cytotoxic T lymphocyte (CTL) response to HCV. For example, a CTL response to an HCV-infected cell can be induced.

In some cases, an effective amount (e.g., a therapeutically effective amount) of an HCV E1/E2 immunogenic composition of the present disclosure, or a nucleic acid(s) comprising nucleotide sequences encoding a heterodimeric polypeptide of the present disclosure, is an amount that, when administered to an individual in one or more doses, is effective to induce a helper T lymphocyte (e.g., CD4⁺ T cell) to HCV in an individual.

In some cases, an effective amount (e.g., a therapeutically effective amount) of an HCV E1/E2 immunogenic composition of the present disclosure, or a nucleic acid(s) comprising nucleotide sequences encoding a heterodimeric polypeptide of the present disclosure, is an amount that, when administered to an individual in one or more doses, is effective to induce an antibody response (e.g., a neutralizing antibody response) and/or a CTL response and/or a helper T cell response to HCV genotype 1. In some cases, an effective amount (e.g., a therapeutically effective amount) of an HCV E1/E2 immunogenic composition of the present disclosure, or a nucleic acid(s) comprising nucleotide sequences encoding a heterodimeric polypeptide of the present disclosure, is an amount that, when administered to an individual in one or more doses, is effective to induce an antibody response (e.g., a neutralizing antibody response) and/or a CTL response and/or a helper T cell response to HCV genotype 3. In some cases, an effective amount (e.g., a therapeutically effective amount) of an HCV E1/E2 immunogenic composition of the present disclosure, or a nucleic acid(s) comprising nucleotide sequences encoding a heterodimeric polypeptide of the present disclosure, is an amount that, when administered to an individual in one or more doses, is effective to induce an antibody response (e.g., a neutralizing antibody response) and/or a CTL response and/or a helper T cell response to HCV genotype 1 and HCV genotype 3. In some cases, an effective amount (e.g., a therapeutically effective amount) of an HCV E1/E2 immunogenic composition of the present disclosure, or a nucleic acid(s) comprising nucleotide sequences encoding a heterodimeric polypeptide of the present disclosure, is an amount that, when administered to an individual in one or more doses, is effective to induce an antibody response (e.g., a neutralizing antibody response) and/or a CTL response and/or a helper T cell response to HCV of any genotype.

An HCV E1/E2 immunogenic composition of the present disclosure, or a nucleic acid(s) comprising nucleotide sequences encoding a heterodimeric polypeptide of the present disclosure, is generally administered in an amount effective to elicit an immune response, e.g., a humoral immune response (e.g., an antibody response) and/or a CTL response, in the mammalian subject. Effective amounts for immunization will vary, and can generally range from about 1 μg to 100 μg per 70 kg patient, e.g., from about 5 μg/70 kg to about 50 μg/70 kg. Substantially higher dosages (e.g. 10 mg to 100 mg or more) may be suitable in oral, nasal, or topical administration routes. The initial administration can be followed by booster immunization of the same HCV E1/E2 immunogenic composition or a different HCV E1/E2 immunogenic composition. In some instances, a subject method of inducing an immune response involves an initial administration of an HCV E1/E2 immunogenic composition of the present disclosure, followed by at least one booster, and in some instances involves two or more (e.g., three, four, or five) boosters. The interval between an initial administration and a booster, or between a give booster and a subsequent booster, can be from about 1 week to about 12 weeks, e.g., from about 1 week to about 2 weeks, from about 2 weeks to about 4 weeks, from about 4 weeks to about 6 weeks, from about 6 weeks to about 8 weeks, from about 8 weeks to about 10 weeks, or from about 10 weeks to about 12 weeks.

In general, immunization can be accomplished by administration of an HCV E1/E2 immunogenic composition of the present disclosure, or a nucleic acid(s) comprising nucleotide sequences encoding a heterodimeric polypeptide of the present disclosure, by any suitable route, including administration of the composition orally, nasally, nasopharyngeally, parenterally, enterically, gastrically, topically, transdermally, subcutaneously, intramuscularly, in tablet, solid, powdered, liquid, aerosol form, locally or systemically, with or without added excipients. Actual methods for preparing parenterally administrable compositions will be known or apparent to those skilled in the art and are described in more detail in such publications as Remington's Pharmaceutical Science, 15th ed., Mack Publishing Company, Easton, Pa. (1980). In some instances, immunization is accomplished by intramuscular injection of an HCV E1/E2 immunogenic composition of the present disclosure.

In some cases, a method of the present disclosure for inducing an immune response to HCV in an individual comprises administering to the individual an effective amount of a nucleic acid(s) comprising nucleotide sequences encoding a heterodimeric polypeptide of the present disclosure. In some cases, the nucleic acid is present in an expression vector. Suitable expression vectors include, but are not limited to, a replication-defective adenovirus vector; a replication-defective vaccinia virus vector; a lentivirus vector (e.g., a self-inactivating lentivirus vector); a retroviral vector (e.g., a self-inactivating retroviral vector); an adeno-associated virus vector; and the like. In some cases, the vector is a modified vaccinia Ankara (MVA) vector, or an MVA-based vector (see, e.g., Verheust et al. (2012) Vaccine 30:2623).

In some cases, a method of the present disclosure for inducing an immune response to HCV in an individual comprises administering to the individual an effective amount of a nucleic acid(s) comprising nucleotide sequences encoding a heterodimeric polypeptide of the present disclosure. In some cases, the nucleic acid is an RNA comprising nucleotide sequences encoding a heterodimeric polypeptide of the present disclosure. See, e.g., Weiner (2013) Molec. Therapy 21:506; and Ulmer et al. (2012) Vaccine 30:4414. In some cases, an RNA (e.g., a single mRNA molecule; or 2 mRNA molecules) comprising nucleotide sequences encoding a heterodimeric polypeptide of the present disclosure is formulated with a liposome. In some cases, an RNA (e.g., a single mRNA molecule; or 2 mRNA molecules) comprising nucleotide sequences encoding a heterodimeric polypeptide of the present disclosure is complexed with protamine. In some cases, an RNA (e.g., a single mRNA molecule; or 2 mRNA molecules) comprising nucleotide sequences encoding a heterodimeric polypeptide of the present disclosure is complexed with 1,2-dioleoyl-3-trimethylammonium-propane/1,2-dioleoyl-sn-glycero-3-phosphoethanolamine (DOTAP/DOPE).

### Individuals Suitable for Administration

Individuals who are suitable for administration with an HCV composition of the present disclosure include immunologically naïve individuals (e.g., individuals who have not been infected with HCV and/or who have not been administered with an HCV vaccine).

Individuals who are suitable for administration with an HCV composition of the present disclosure include individuals who are at greater risk than the general population of becoming infected with HCV, where such individuals include, e.g., intravenous drug users; individuals who are the recipients, or the prospective recipients, of blood or blood products from another (donor) individual(s); individuals who are the recipients, or the prospective recipients, of non-autologous cells, tissues, or organs from another (donor) individual; health care workers; emergency medical and non-medical personnel (e.g., first responders; fire fighters; emergency medical team personnel; etc.) and the like.

Individuals who are suitable for administration with an HCV E1/E2 immunogenic composition of the present disclosure include individuals who recently became exposed to HCV or who recently became infected with HCV. For example, a subject immunogenic composition can be administered to an individual within from about 24 hours to about 48 hours, from about 48 hours to about 1 week, or from about 1 week to about 4 weeks, following possible or suspected exposure to HCV or following infection with HCV.

Individuals who are suitable for administration with an HCV E1/E2 immunogenic composition of the present disclosure include individuals who have been diagnosed as having an HCV infection, and include chronically infected individuals. In some cases, an individual who has been diagnosed as having an HCV infection is treated with an anti-viral agent and a subject HCV immunogenic composition. Suitable anti-viral agents for treating HCV infection include, e.g., ribavirin (1-β-D-ribofuranosyl-1H-1,2,4-triazole-3-carboxamide); interferon-alpha (IFN-α) (where “IFN-α” includes IFN-α2a; IFN-α2b; IFN-α that is conjugated with poly(ethylene glycol) (“pegylated IFN-α), where the pegylated IFN-α can be pegylated IFN-α2a or pegylated IFN-α 2b); an HCV NS3 protease inhibitor (e.g., boceprevir; telaprevir); and an HCV NS5 protease inhibitor. In some cases, an individual who has been diagnosed as having an HCV infection is treated with, e.g.: 1) IFN-α+ribavirin; and a subject HCV immunogenic composition; or 2) IFN-α+ribavirin+an HCV protease inhibitor (e.g., boceprevir or telaprevir); and a subject HCV immunogenic composition. Suitable anti-viral agents for treating HCV infection include Sovaldi (Sofosbuvir; a nucleotide analog that functions as an NS5B polymerase inhibitor), alone or in combination with pegylated IFN-α and ribavirin. Suitable anti-viral agents for treating HCV infection include Sovaldi. Suitable anti-viral agents for treating HCV infection include Harvoni® (ledipasvir 90 mg+sofosbuvir 400 mg). Ledipasvir is an HCV NS5A inhibitor. Harvoni® can be administered with or without ribavirin.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular (ly); i.p., intraperitoneal (ly); s.c., subcutaneous (ly); and the like

### Example 1: Production of Affinity Tagged HCV E1/E2 Heterodimers, and Production and Characterization of Untagged HCV E1/E2 Heterodimers

FIG. 6A. Schematic representation of Fc tag insertion to E2 N-terminus in full length E1E2 polypeptide construct. The E1E2 polypeptide is expressed under the control of the CMV promoter (P_{CMV}) and includes the signal sequence from tissue plasminogen activator (tPA). Insertion sites are shown for representative HCV E1E2 sequences: H77 (GenBank NP_671941), JFH1 (Genbank ABO47639; genotype 2A), S52 (Genbank ADF97232.1; genotype 3a), and isolate QC69 (Genbank: ABN05226.1; genotype 7A). Sizes of the polypeptide regions are shown at the top (aa=amino acids). At the N-terminus of E2, a duplication of the E2 N-terminal amino acids respective to the particular genotype (eg: ET addition for H77; GenBank NP_671941) is inserted followed by the human IgG1 Fc tag (hu IgG1 Fc) and a PreScission Protease (PP) recognition sequence (LEVLFQGP; SEQ ID NO:1). Depending on the genotype and specific isolate of E1E2, duplication of the first two amino acids of E2 may result in an undesirable amino acid created at the N-terminus of E2 following processing by signal peptidase (SP) (FIG. 6A). Such amino acids at the amino terminus include asparagine (N), glutamine (Q) or cysteine (C). Such amino acids can target the protein for proteasome-mediated degradation via the N-end rule pathway (reviewed in: Tasaki T et al. 2012. Annu Rev Biochem 81 261-289). In this case, an alternative amino acid could be selected according to either the consensus sequence for the particular genotype or a particular genotype subclass would be selected. Following expression of the polypeptide, signal peptidase (SP) cleavages result in the downstream E1 and E2 polypeptides shown. The E1 and E2 polypeptides interact to form a heterodimer. For purification purposes, the Fc tagged E1E2 is immobilized on Protein A or Protein G resin and digested with PreScission Protease (PP) (cleavage between Q and G in the LEVLFQGP (SEQ ID NO: 1) sequence) to release the untagged E1E2 heterodimer.

FIG. 6B. Purification of E1E2 heterodimer from CHO cell extracts expressing Fc tagged E1E2 H77. CHO cell extracts expressing an Fc-tagged E1E2 H77 construct, as diagramed in (A), were immobilized on Protein-G Sepharose 4 Fast Flow and digested with GST-PreScission protease (GST-PP) to remove the Fc tag. Following the digestion with GST-PP and release of untagged E1E2, GST-PP was removed by Glutathione Sepharose 4B. The untagged E1E2 protein concentrate was then applied to a CHT™ ceramic hydroxyapatite ((Ca₅ (PO₄)₃OH)₂) type I (HAP) column. The HAP flow through containing the final E1E2 heterodimer was collected and samples loaded onto a sodium dodecyl sulfate-polyacrylamide gel electrophoresis (SDS-PAGE) gel. After electrophoresis, the gel was stained, or blotted then probed with antibodies. (1) Western blot with anti-E1 (A4) and anti-E2 (H52) monoclonal antibodies (mAbs) (0.5 μg loaded per lane). (2) Colloidal Coomassie Brilliant Blue G250 stained gel (2 μg loaded per lane).

FIG. 7. Alignment of the Fc-tagged E1-E2 polypeptide for H77 and Alberta isolate Avi1a129 (genotype 1A). The amino acid sequence for the coding region of the tPa-E1-Fc-PP-E2 construct (as diagrammed in FIG. 6A) for the Alberta isolate (Avi1a129) and H77 (GenBank NP_671941) was aligned using Geneious software v5.6.4. Arrowhead denotes the insertion of the duplicated N-terminal E2 residues that precede the Fc-PP tag (Avi1a129: QT and H77: ET).

FIG. 8. Alignment of the Fc-tagged E1-E2 polypeptide for S52 (Genbank ADF97232.1) and Alberta isolate Avi3a177 (genotype 3A). The amino acid sequence for the coding region of the tPA-E1-Fc-PP-E2 construct (as diagrammed in FIG. 6A) for the Alberta isolate (Avi3a177) and S52 was aligned using Geneious software v5.6.4. Arrowhead denotes the insertion of the duplicated N-terminal E2 residues that precede the Fc-PP tag (ET for both Avi3a177 and S52).

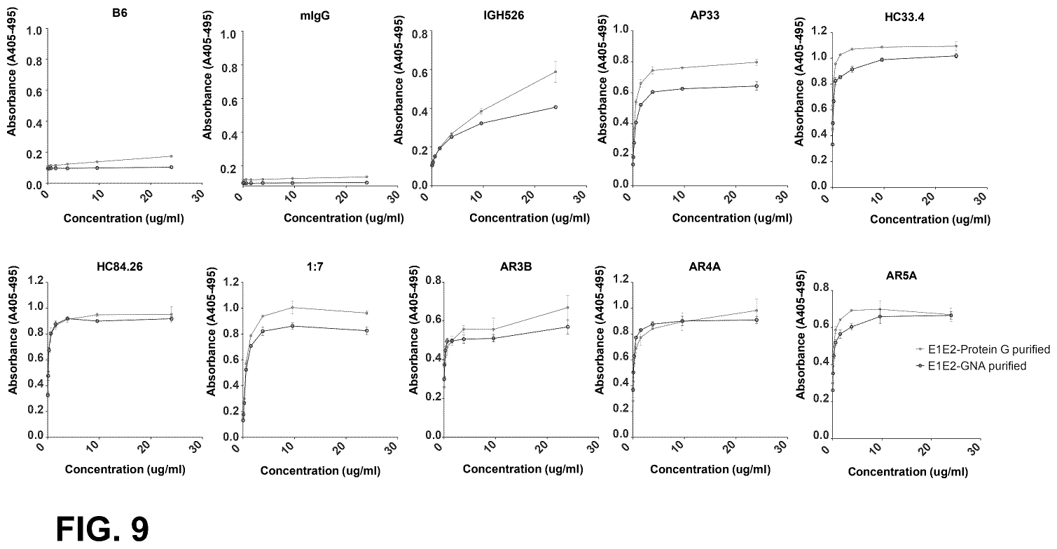

FIG. 9. Binding of HCV neutralizing monoclonal antibodies (mAb) to recombinant E1E2 antigens. Recombinant E1E2 (H77) antigens were purified by Galanthus nivalis agarose (GNA agarose) (untagged E1E2; pink closed circle) or Protein G Sepharose (Fc-tagged E1E2 followed by Fc tag removal; purple open circle). To determine proper folding of the E1E2 antigens, a panel of monoclonal antibodies (mAb) established to neutralize HCV infectivity were examined by enzyme-linked immunosorbent assay (ELISA). Recombinant E1E2 antigens were coated to ELISA plates and increasing amounts of eight different neutralizing HCV mAbs directed to E1 (IGH526), E2 (AP33, HC33.4, HC84.26, 1:7, AR3B) or E1E2 (AR4A, AR5A) were used. Clone B6 (human IgG1) and mouse IgG1 were used as negative controls. Results show that E1E2 antigens isolated by both methods show equal affinity to the panel of tested mAbs.

### Example 2: Characterization of Affinity Tag-Derived HCV E1/E2 Heterodimer

### Materials and Methods

### Cell Cultures and Antibodies

Chinese hamster ovary (CHO) cells stably expressing recombinant E1E2 constructs from the genotype 1a H77c strain (GenBank accession number AF009606) were propagated in Iscove's modified Dulbecco's medium (Thermo Fisher Scientific, Waltham, MA, USA) containing 10% heat-inactivated fetal bovine serum (FBS) (Thermo Fisher Scientific), 0.1 mM/0.016 mM sodium hypoxanthine/thymidine (HT supplement; Thermo Fisher Scientific), 0.002 mM methotrexate, 100 units/mL penicillin, and 100 μg/mL streptomycin (PenStrep; Invitrogen, Carlsbad, CA, USA). Huh-7.5 cells were propagated in Dulbecco's modified Eagle's medium (Thermo Fisher Scientific) containing 10% heat-inactivated fetal bovine serum (Omega Scientific, Tarzana, CA, USA), 0.1 mM nonessential amino acids (Invitrogen), and penicillin and streptomycin (PenStrep; Invitrogen). The mAb mouse anti-cluster of differentiation 81 (CD81) clone JS-81 (BD Biosciences, Franklin Lakes, NJ, USA), mouse isotype control IgG1 (R&D Systems, Minneapolis, MN, USA), anti-HCV mAbs (HC33.4, HC84.26, AR3B, AR4A, and AR5A), and human anti-HIV antibody B6 have been described.

### Expression and Purification of Recombinant E1E2 Antigens

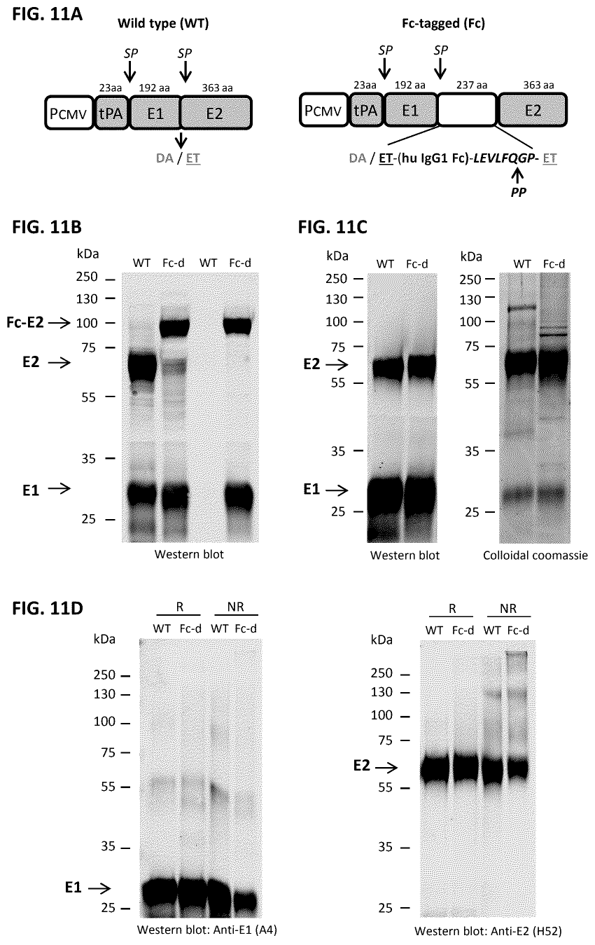

The E1E2 glycoprotein coding region from H77c (genotype 1a) (Genbank AF009606; amino acids 192-746), preceded by the signal peptide sequence for tissue plasminogen activator (tPA), was inserted into the SpeI/MluI site of the pTRIP lentiviral vector bearing an IRES-AcGFP reporter. For the Fc-tagged E1E2 construct, a duplication of amino acids 384-385 (ET) was inserted at the N-terminus of E2 followed by the human IgG1 Fc tag (227 amino acids) and a PreScission Protease/human rhinovirus protease 3C (HRV3C) sequence (LEVLFQGP; SEQ ID NO: 1) (FIG. 11A). Lentiviral particles were generated in HEK-293 T cells according to a previous method (Schoggins et al. (2011) Nature 472:481), and CHO cells were transduced with packaged lentivirus. GFP-positive CHO cells expressing WT or Fc-tagged recombinant E1E2 were sorted by flow cytometry using a BD FACSAria III cell sorter (BD Biosciences) then suspension adapted in PROCHO4 medium (Lonza, Walkersville, MD, USA) with 6% FBS in 250 ml shaker flasks (Corning, Corning, NY, USA) and expanded in 3 L spinner flasks (Corning, Corning, NY, USA).

Recombinant WT E1E2 was purified from CHO cell extracts using GNA (Vector laboratories, Burlingame, CA, USA) according to a previous study (Ralston et al. (1993) J. Virol. 67:6753). The GNA eluate fraction was loaded on to a hydroxyapatite (HAP) column (BIO-RAD™, Hercules, CA, USA; 158-8000) and the flow through concentrated with a 50,000 molecular weight cut-off centrifugal filter unit (EMD MILLIPORE™, Billerica, MA, USA). For Fc-d E1E2, the CHO cell extract was applied to Protein G Sepharose 4 Fast Flow (GE Healthcare, Piscataway, NJ, USA), washed with 10 mM sodium phosphate, 80 mM NaCl, 0.1% TX-100, pH 6.8, and the resin digested with His6-GST-HRV3C protease (Thermo Fisher Scientific) overnight at 4° C. The digested material was applied to Glutathione Sepharose 4B (GE Healthcare) to remove the protease and the flow through applied to HAP with final concentration as described for WT E1E2.

### Immunization of Mice and Serum Samples

Female CB6F1 mice (Charles River Laboratories, Montreal, QC, Canada) (5-7 weeks old) used for vaccination experiments were cared for in accordance with the Canadian Council on Animal Care guidelines. Experimental methods were reviewed and approved by the University of Alberta Health Sciences Animal Welfare Committee. Recombinant E1E2 H77 antigens (2 μg) were mixed in a 1:1 ratio with 75 μg alum and 7.5 μg monophosphoryl Lipid A (MPLA Vaccigrade) (Invivogen, San Diego, CA, USA) (30 μL final injection volume). Mice were injected intramuscularly at days 0, 14, and 28. Pre-vaccination serum was collected at day 0 and post vaccination sera (terminal bleeds) obtained at day 42. Sera were collected after centrifugation of the samples at 5000 g for 15 minutes. Sera were heat-inactivated by incubation at 56° C. for 30 minutes and stored in aliquots at −80° C. until use.

### ELISA

(i) E1E2 ELISA. Microtiter plates (CORNING) were coated with E1E2 antigens (100 ng/well) in carbonate buffer (15 mM sodium carbonate, 35 mM sodium bicarbonate, pH 9.6) overnight at 4° C. Plates were washed with phosphate-buffered saline containing 0.2% TWEEN 20 (PBST) and blocked for 1 h in 4% bovine serum albumin (SIGMA-ALDRICH, St. Louis, MO, USA) in PBST. E2-specific mAbs (HC33.4, HC84.26, and AR3B) (Law et al. (2008) Nat. Med. 14:25; Keck et al (2013) J. Virol. 87:37; and Keck et al. (2012) PLOS Pathogens 8: e1002653), E1E2-specific mAbs (AR4A and AR5A) (15), or control (B6) (16) mAb (50 μL/well) were added for 1 h and detected by an anti-human alkaline phosphatase-conjugated secondary antibody (1:10,000; Jackson Immuno Research, West Grove, PA, USA) and p-nitrophenyl phosphate (MilliporeSigma) substrate. Absorbance (405-495 nm) was read using an Enspire plate reader (Perkin-Elmer, Waltham, MA, USA).

(ii) E2 ELISA. Microtiter plates were coated with E2 (amino acids 384-661) HCV1 (Genbank M62321.1) and blocked in 4% bovine serum albumin as described for the E1E2 ELISA. Antisera from vaccinated mice (terminal bleeds) were diluted in PBST and added to the plates for 1 h (50 μL/well). E2-specific antibodies from mouse antisera were detected by a horseradish peroxidase-conjugated goat anti-mouse secondary antibody (1:10,000; Cedarlane Laboratories, Burlington, ON, Canada) and peroxidase substrate (KPL, Gaithersburg, Md, USA). Absorbance was read at 450-570 nm. Absorbance values from three independent experiments are expressed as means±SEM.

(iii) Competition ELISA. Mouse antisera (terminal bleeds) were assessed for competition with conformation-specific E2 mAbs for E1E2 binding according to a method described previously (Wong et al. (2014) J. Virol. 88:14278). Briefly, microtiter plates (Corning) were coated with GNA purified WT E1E2 H77c in carbonate buffer overnight at 4° C. and blocked in 1% casein (Sigma-Aldrich) in PBST. Diluted mouse antisera were incubated for 1 h in E1E2-coated wells. HCV-specific (HC33.4, HC84.26, AR3B, AR4A, and AR5A) or control (B6 anti-HIV) mAbs were added at a concentration normally resulting in 70% maximal binding. Binding of HCV-specific mAbs was detected by an anti-human alkaline phosphatase-conjugated secondary antibody (1:10,000; Jackson Immuno Research) and p-nitrophenyl phosphate (MilliporeSigma) substrate. Absorbance was read at 405-495 nm. Values were calculated as the percentage of mAb binding relative to the mAb bound in the absence of antiserum. Data were plotted as means±standard error of the mean from three independent experiments.

### HCV Pseudotype Virus (HCVpp) Production and Neutralization Assay

HCVpp expressing a luciferase reporter were generated as described previously (Hsu et al. (2003) Proc. Natl. Acad. Sci. USA 100:7271). For neutralization assays, human hepatoma cells (Huh7.5) were plated on poly-lysine coated 96-well plates 1 day prior to infection. HCVpp were diluted 1:10 and premixed with heat inactivated diluted sera for 1 h at 37° C. followed by addition to Huh 7.5 cells. Six h post-infection, the antibody-virus inoculum was replaced with fresh culture medium. Cells were processed 48 h post-infection using the Bright-glo luciferase assay system (Promega, Madison, WI, USA). Luminescence was measured using an Enspire plate reader (Perkin Elmer). Neutralization activity was calculated using the following formula: % neutralization=(pre-post)/pre×100 where pre/post represent the luciferase activity done after incubating with either the pre- or post-vaccination sera. For ID50 titres, 2-fold dilutions of sera from 1:25 to 1:1600 were examined. Titres were expressed as a reciprocal of the dilution calculated to neutralize 50% of the virus. If 50% neutralization was not achieved at the highest concentration examined (1:25), then the next highest concentration was assigned to the sample (ie: 1:12.5). Similarly, if the lowest dilution examined (1:1600) resulted in >50% neutralization, we assigned the next dilution for this sample (ie: 1:3200).

### Results

The results are depicted in FIGS. 11-14.

Purification of E1E2 from an Fc-Tagged Precursor

The constructs used for E1E2 expression are shown in FIG. 11A. The full length E1E2 sequence from H77c (Genbank AF009606) was inserted downstream of the tPA leader sequence and a CMV promoter. To generate an affinity tagged form of E1E2, the Fc domain from human IgG1 was inserted at the junction between E1 and E2. Both WT and Fc-E1E2 could be precipitated by GNA, whereas only the latter was precipitated by Protein G Sepharose (FIG. 11B). Importantly, the precipitated Fc-E2 protein was associated with E1, demonstrating that the Fc tag did not interfere with heterodimer formation.

As outlined in Materials and Methods, above, Fc-d E1E2 (with the Fc tag removed) was purified and compared to WT E1E2 isolated with GNA. Both methods resulted in a highly enriched E1E2 antigen that contained a minor fraction of contaminant proteins (FIG. 11C). The yields between the two methods were similar at the scale of the isolations performed (˜1 mg E1E2 per 100 g CHO cells). SDS-PAGE and western blot analyses performed with reducing vs. non-reducing Laemmli buffer supported that the vast majority of the isolated E1E2 antigens were in non-covalently linked complexes (FIG. 11D).

FIG. 11A-11D: Purification of E1E2 from an Fc-tagged precursor. (A). Schematic representation of wild type (WT) and Fc-tagged constructs and polypeptide processing. The E1E2 H77c (Genbank AF009606) polypeptide was expressed under the control of the CMV promoter (P_{CMV}) and preceded by the signal sequence from tissue plasminogen activator (tPA). For Fc-tagged E1E2, a duplication of the E2 N-terminal amino acids (384-385) (ET) was inserted followed by the human IgG1 Fc tag (hu IgG1 Fc) and a PreScission Protease (PP) recognition sequence (LEVLFQGP; SEQ ID NO:1). Sizes of the polypeptide regions are shown at the top (aa=amino acids) as well as cleavage sites by signal peptidase (SP). (B) Capture of wild-type (WT) or Fc tag derived (Fc-d) E1E2 from CHO cell extracts was performed with GNA and Protein G Sepharose, respectively, and proteins separated by SDS-PAGE and blotted with anti-E1 (A4) and anti-E2 (H52) monoclonal antibodies (mAbs) (C) Purified E1E2 antigens (with PP-mediated Fc tag removal step for Fc-d) were separated by SDS-PAGE. Left panel: Western blot: anti-E1 (A4) and anti-E2 (H52) mAbs (1 μg load per lane). Right panel: Coomassie Brilliant Blue G250 (2 μg load per lane). (D) Wild type (WT) and Fc tag derived (Fc-d) E1E2 antigens (1 μg/lane) were denatured at 95° C. for 5 minutes in laemelli buffer with (R) or without (NR) 1% β-mercaptoethanol. Samples were separated by SDS-PAGE and blotted with ant-E1 (A4) and anti-E2 (H52) mAbs.

Binding of HCV Cross Neutralizing Monoclonal Antibodies (mAb) to Purified E1E2 Heterodimers

E1E2 antigens were coated to ELISA plates and probed using E2-specific (HC33.4, HC84.26, and AR3B) and E1E2-specific (AR4A and AR5A) cross-neutralizing mAbs. HC33.4, HC84.26, and AR3B target the disparate regions of E2 that form the CD81 receptor binding site (CD81bs) and are capable of binding soluble E2. HC33.4 recognizes a linear epitope in E2 (aa 409-423). AR3B and HC84.26 are directed to conformation-specific E2 epitopes. However, HC84.26 additionally recognizes an E2 linear synthetic peptide (aa 433-447). AR4A and AR5A recognize unique conformational epitopes outside of the CD81bs and are capable of binding native E1E2 but not E2 or E1 alone. WT and Fc-d E1E2 antigens bound E2- and E1E2-specific antibodies in a nearly identical manner. These data support that, similar to WT, Fc-d E1E2 maintains correct folding and presents conformation-specific E2 and E1E2 cross-neutralizing epitopes.

The Fc-d E1E2 Heterodimer Elicits Neutralizing Antibodies (nAbs) in Mice

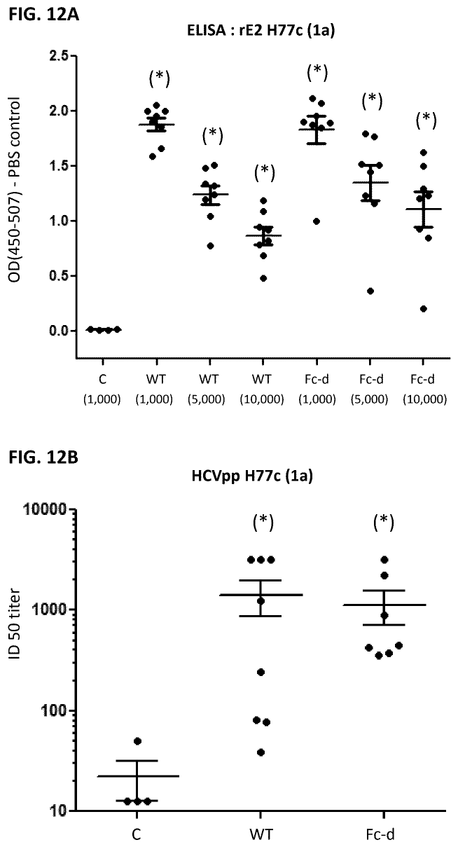

Eight CB6F1 mice were immunized with WT or Fc-d E1E2 (2 μg per injection) in a 1:1 ratio with adjuvant (75 μg alum and 7.5 μg MPLA). Four animals were injected with buffer containing adjuvant as controls. Animals received a total of three injections at 2 week intervals. Sera obtained from terminal bleeds were examined for homologous anti-E2 activity by ELISA plates coated with recombinant E2 HCV1 (1a). WT and Fc-d E1E2 vaccinated sera exhibited strong anti-E2 titers for E2 HCV1 at all dilutions examined (1000-, 5000-, and 10,000-fold) (FIG. 12A). For each dilution examined, sera from mice vaccinated with WT and Fc-d E1E2 were statistically elevated from controls (p<0.05; one-way ANOVA; Tukey's post-hoc test) (FIG. 12A). Mean absorbance values between sera from mice vaccinated with WT and Fc-d E1E2 exhibited no statistical difference at each dilution examined.

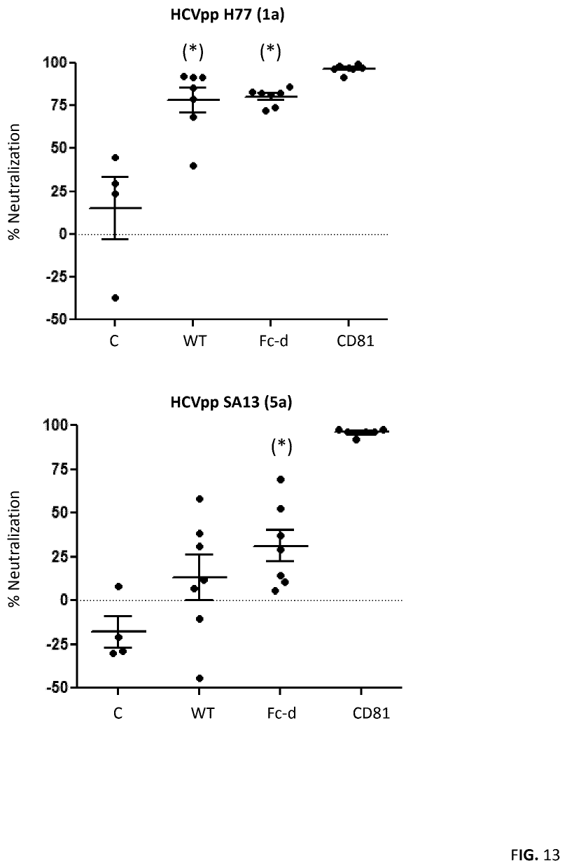

Pre- and post-vaccination serum samples were evaluated for their ability to inhibit the infectivity of HCVpp H77 in Huh7.5 cells. Two-fold serial dilutions (1:25 to 1:1600) of sera were examined and the inhibitory dose to achieve 50% neutralization (ID50) represented as the reciprocal value of the dilution (FIG. 12B). WT and Fc-D E1E2 exhibited similar group mean ID50 values of 1412±541 and 1134±428, respectively, that were each significant from the control group. Fc-d vaccinated mice exhibited less variability in ID50 values (min 360; max 3200) than the WT group (min 39; max 3200). The neutralization response of homologous genotype 1a (H77) to heterologous genotype 5a (SA13) HCVpp was compared. At 1:50 diluted sera WT and Fc-d groups showed very similar mean neutralizations of HCVpp H77 at 78.3±7.2% and 80.2±2.0%, respectively (FIG. 13). Fc-d vaccinated animals exhibited a significant neutralization of HCVpp SA13 (mean 31±9%) from controls. However, the WT group did not show neutralization towards SA13 that was significant from the control group (FIG. 13). No neutralization activity within the Fc-d group using further dilutions of the sera was detected.

FIG. 12A-12B: Neutralizing antibodies induced by vaccination with wild type (WT) and Fc-tag. (A) Recombinant E2 HCV1 (la) (amino acids 384-661; Genbank M62321.1) was coated to ELISA plates in triplicate and probed with post-vaccinated mice sera. Binding of E2-specific antibodies in WT and Fc tag-derived (Fc-d) E1E2 vaccinated mice sera (1000, 5000, 10,000-fold dilutions) compared to control vaccinated sera (1000-fold dilution) were detected by anti-mouse horse radish peroxidase (HRP) conjugated secondary antibody and peroxidase substrate. The OD450-507 nm values (means and SEM) from three independent experiments are shown plotted vs serum dilution. (B) Neutralization of HCVpp H77c (la) entry was performed using 2-fold dilutions (1:25 to 1:1600) of pre- and post-vaccinated sera and the ID50 values determined (shown as the reciprocal value of the dilution to achieve 50% neutralization). The group mean with SEM is shown from a representative of two independent experiments. Vaccinated mice groups: Control (C): buffer+alum/MPL; WT (GNA agarose-derived E1E2 H77c+alum/MPL; Fc-d (Fc tag-derived E1E2 H77c+alum/MPL). (*) designates p<0.05 respective to control (C) by One way ANOVA; Kruskal Wallis, Dunn's post-test.

FIG. 13: Comparison of neutralizing antibodies towards homologous (la) and heterologous (5a) HCVpp. Neutralization of homologous HCVpp H77 (1a) and heterologous HCVpp SA13 (5a) was performed using pre- and post-vaccinated sera (1:50) and the group means with SEMs plotted from representatives of two independent experiments. Positive control: Anti-CD81 mAb (1 μg/ml). (*) designates p<0.05 respective to control (C) by One way ANOVA; Tukey post-test.

Antisera from E1E2-Vaccinated Mice Compete with Cross-Neutralizing HCV mAbs for Binding to E1/E2

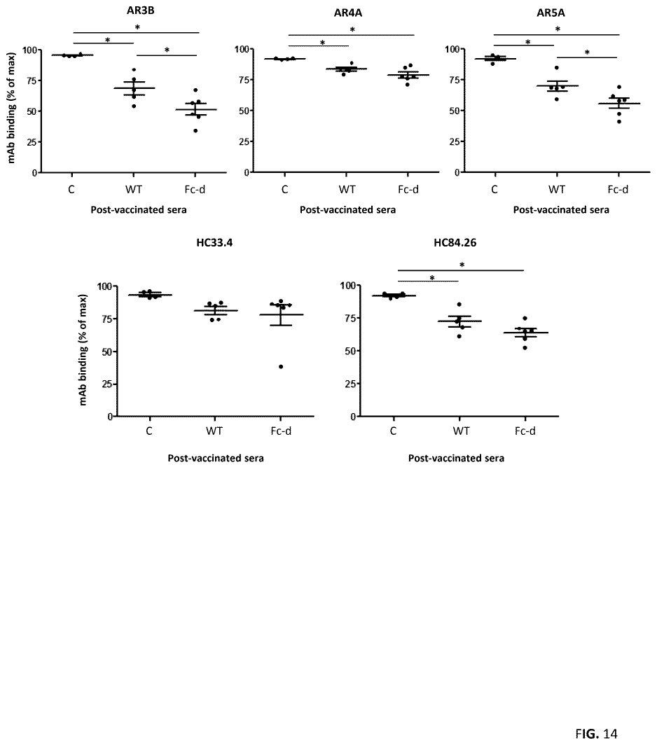

The observed neutralization responses of WT and Fc-d vaccinated mice for HCVpp H77 (1a) and HCVpp SA13 (5a) indicated a dominance towards homologous (1a) E1E2 epitopes. To determine if anti-E1E2 antibodies from WT and Fc-d E1E2 vaccinated animals targeted conserved epitopes to well-characterized HCV neutralizing mAbs, competition ELISA assays were performed according to Wong et al. (2014) infra. Antisera from control animals exhibited negligible competition with each of the mAbs examined: >90% mAb binding (normalized to maximum binding in the absence of any sera) (FIG. 14). Vaccination with WT and Fc-d E1E2 significantly reduced AR3B, AR4A, AR5A, and HC84.26 binding compared to control sera (p<0.05; one-way ANOVA; Tukey's post-hoc test). For HC33.4, neither the WT nor Fc-d E1E2 groups impaired the binding of this mAb significantly compared to controls.

For both the AR4A and HC84.26 mAbs, binding was reduced to a similar extent in WT and Fc-derived E1E2 vaccinated animals (AR4A: mean 83.4 and 78.8%; HC84.26: mean 72.3 and 63.8% for WT and Fc groups, respectively). For AR3B and AR5A, the Fc-d E1E2 vaccinated group impaired mAb binding to a greater extent than WT vaccinated animals (AR3B: mean 68.6 and 51.5%; AR5A: mean 69.8 and 55.9% for WT and Fc groups, respectively) (p<0.05; one-way ANOVA; Tukey's post-hoc test). These findings suggest that WT and Fc-d mice did elicit anti-HCV antibodies that target conserved neutralizing epitopes. However, it is noted that our competition ELISA likely exhibits high sensitivity since the neutralization of heterologous HCVpp SA13 (5a) was considerably lower compared to HCVpp H77 (1a) from WT and Fc-d group sera (FIG. 13).

FIG. 14. Mice antisera compete for the binding of HCV cross-neutralizing monoclonal antibodies (mAb) to E1E2. Competition studies with mouse antisera and a panel of cross-neutralizing human HCV antibodies. Microtiter wells containing GNA-purified E1E2 H77c were incubated with diluted post-vaccination antiserum (1:100) in triplicate for 1 h followed by incubation with the indicated mAb. Binding of the mAbs was detected with anti-human alkaline phosphatase-conjugated secondary antibodies. The percentages of mAb binding were calculated relative to the amount of mAb bound in the absence of antiserum. Shown are mean values for each group±range from two independent experiments. Vaccinated mice groups: Control (C): buffer+alum/MPL; WT (GNA agarose-derived E1E2 H77c+alum/MPL; Fc-d (Fc tag-derived E1E2 H77c+alum/MPL). E2-specific antibodies: HC33.4, HC84.26, AR3B. E1E2-specific antibodies: AR4A, AR5A. (*) designates p<0.05 by one-way ANOVA; Tukey's post-hoc test. With n=2 SEM is actually the same as the range.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1.-80. (canceled)

81. A nucleic acid comprising a nucleotide sequence encoding a polyprotein comprising, in order from N-terminus to C-terminus:
a) an HCV E1 polypeptide; and
b) an HCV E2-affinity tag fusion polypeptide comprising, in order from N-terminus to C-terminus: i) an affinity tag polypeptide; ii) a proteolytically cleavable linker; and iii) an HCV E2 polypeptide.

82. The nucleic acid of claim 81, wherein the nucleotide sequence encoding the polyprotein is operably linked to a promoter functional in an eukaryotic cell.

83. The nucleic acid of claim 81, wherein the affinity tag polypeptide is an Ig Fc polypeptide.

84. The nucleic acid of claim 83, wherein the Ig Fc polypeptide comprises an amino acid sequence having at least 85% amino acid sequence identity to an amino acid sequence set forth in any one of SEQ ID NOs: 102-109.

85. The nucleic acid of claim 81, wherein the proteolytically cleavable linker comprises an amino acid sequence selected from the group consisting of LEVLFQGP as set forth in SEQ ID NO: 1, ENLYFQS as set forth in SEQ ID NO:2, DDDDK as set forth in SEQ ID NO:3, I(E/D)GR as set forth in SEQ ID NO:45, and LVPRGS as set forth in SEQ ID NO:5.

86. The nucleic acid of claim 81, wherein the HCV E1 polypeptide has a length of from about 175 amino acids to about 195 amino acids, the affinity tag polypeptide comprises an Ig Fc polypeptide, the proteolytically cleavable linker comprises an amino acid sequence selected from the group consisting of LEVLFQGP as set forth in SEQ ID NO:1, ENLYFQS as set forth in SEQ ID NO:2, DDDDK as set forth in SEQ ID NO:3, I(E/D)GR as set forth in SEQ ID NO:45, and LVPRGS as set forth in SEQ ID NO: 5, and the HCV E2 polypeptide has a length of from about 350 amino acids to about 370 amino acids.

87. The nucleic acid of claim 81, wherein the HCV E1 polypeptide comprises an amino acid sequence having at least 95% amino acid sequence identity to an E1 polypeptide sequence set forth in any one of SEQ ID NOs: 46-100.

88. The nucleic acid of claim 81, wherein the HCV E2 polypeptide comprises an amino acid sequence having at least 95% amino acid sequence identity to an E2 polypeptide sequence set forth in any one of SEQ ID NOs: 46-100.

89. The nucleic acid of claim 81, wherein the HCV E1 polypeptide is an AVI1a129 E1 polypeptide comprising the AVI1a129 E1 polypeptide sequence set forth in SEQ ID NO:117, an H77 E1 polypeptide comprising the H77 E1 polypeptide sequence set forth in SEQ ID NO:118, an S52 E1 polypeptide comprising the S52 E1 polypeptide sequence set forth in SEQ ID NO:119, or an AVI3a177 E1 polypeptide comprising the AVI3a177 E1 polypeptide sequence set forth in SEQ ID NO:120.

90. The nucleic acid of claim 81, wherein the HCV E2 polypeptide is an AVI1a129 E2 polypeptide comprising the AVI1a129 E2 polypeptide sequence set forth in SEQ ID NO:117, an H77 E2 polypeptide comprising the H77 E2 polypeptide sequence set forth in SEQ ID NO:118, an S52 E2 polypeptide comprising the S52 E1 polypeptide sequence set forth in SEQ ID NO:119, or an AVI3a177 E2 polypeptide comprising the AVI3a177 E1 polypeptide sequence set forth in SEQ ID NO:120.

91. The nucleic acid of claim 81, wherein the HCV E1 polypeptide and the HCV E2 polypeptide are of the same genotype.

92. The nucleic acid of claim 81, wherein the HCV E1 polypeptide and the HCV E2 polypeptide are of different genotypes.

93. The nucleic acid of claim 81, wherein the HCV E1 polypeptide is of genotype 1, 2, 3, 4, 5, 6, or 7.

94. The nucleic acid of claim 81, wherein the HCV E2 polypeptide is of genotype 1, 2, 3, 4, 5, 6, or 7.

95. The nucleic acid of claim 81, wherein the polyprotein comprises a signal peptide linked to an N-terminus of the HCV E1 polypeptide.

96. The nucleic acid of claim 95, wherein the signal peptide comprises an amino acid sequence from amino acid position 170 through amino acid position 191 of any one of SEQ ID NOs: 46-101

97. The nucleic acid of claim 96, wherein the polyprotein comprises a dipeptide selected from the group consisting of QT, ET, HT, GT, TT, RH, NT, AY, VI, or ST linked to an N-terminus of the affinity tag polypeptide.

98. The nucleic acid of claim 97, wherein the HCV E1 polypeptide is an the H77 E1 polypeptide and the HCV E2 polypeptide is an H77 E2 polypeptide.

99. A viral vector comprising the nucleic acid of claim 81.

100. A genetically modified CHO cell comprising the nucleic acid of claim 81.


SEQUENCE LISTING (123 sequences)

SEQ ID NO: 1 - protein, 8 aa, synthetic construct
LEVLFQGP

SEQ ID NO: 2 - protein, 7 aa, synthetic construct
ENLYFQS

SEQ ID NO: 3 - protein, 5 aa, synthetic construct
DDDDK

SEQ ID NO: 4 - protein, 4 aa, synthetic construct
LVPR

SEQ ID NO: 5 - protein, 6 aa, synthetic construct
LVPRGS

SEQ ID NO: 6 - protein, 23 aa, synthetic construct
MDAMKRGLCCVLLLCGAVFVSPS

SEQ ID NO: 7 - protein, 6 aa, synthetic construct
LEVLFQ

SEQ ID NO: 8 - protein, 6 aa, synthetic construct
ENLYFQ

SEQ ID NO: 9 - protein, 5 aa, synthetic construct
GSGGS

SEQ ID NO: 10 - protein, 4 aa, synthetic construct
GGGS

SEQ ID NO: 11 - protein, 4 aa, synthetic construct
GGSG

SEQ ID NO: 12 - protein, 5 aa, synthetic construct
GGSGG

SEQ ID NO: 13 - protein, 5 aa, synthetic construct
GSGSG

SEQ ID NO: 14 - protein, 5 aa, synthetic construct
GSGGG

SEQ ID NO: 15 - protein, 5 aa, synthetic construct
GGGSG

SEQ ID NO: 16 - protein, 5 aa, synthetic construct
GSSSG

SEQ ID NO: 17 - protein, 6 aa, synthetic construct
  VARIANT 2: note = X may be Leu or Gln.
PXGMTS

SEQ ID NO: 18 - protein, 5 aa, synthetic construct
  VARIANT 2: note = X may be Leu or Gln.
PXGMT

SEQ ID NO: 19 - protein, 10 aa, synthetic construct
CGLVPAGSGP

SEQ ID NO: 20 - protein, 12 aa, synthetic construct
SLLKSRMVPNFN

SEQ ID NO: 21 - protein, 12 aa, synthetic construct
SLLIARRMPNFN

SEQ ID NO: 22 - protein, 12 aa, synthetic construct
SKLVQASASGVN

SEQ ID NO: 23 - protein, 12 aa, synthetic construct
SSYLKASDAPDN

SEQ ID NO: 24 - protein, 12 aa, synthetic construct
RPKPQQFFGLMN

SEQ ID NO: 25 - protein, 12 aa, synthetic construct
SLRPLALWRSFN

SEQ ID NO: 26 - protein, 12 aa, synthetic construct
SPQGIAGQRNFN

SEQ ID NO: 27 - protein, 14 aa, synthetic construct
DVDERDVRGFASFL

SEQ ID NO: 28 - protein, 12 aa, synthetic construct
SLPLGLWAPNFN

SEQ ID NO: 29 - protein, 12 aa, synthetic construct
SLLIFRSWANFN

SEQ ID NO: 30 - protein, 12 aa, synthetic construct
SGVVIATVIVIT

SEQ ID NO: 31 - protein, 12 aa, synthetic construct
SLGPQGIWGQFN

SEQ ID NO: 32 - protein, 12 aa, synthetic construct
KKSPGRVVGGSV

SEQ ID NO: 33 - protein, 12 aa, synthetic construct
PQGLLGAPGILG

SEQ ID NO: 34 - protein, 31 aa, synthetic construct
HGPEGLRVGFYESDVMGRGHARLVHVEEPHT

SEQ ID NO: 35 - protein, 12 aa, synthetic construct
GPQGLAGQRGIV

SEQ ID NO: 36 - protein, 12 aa, synthetic construct
GGSGQRGRKALE

SEQ ID NO: 37 - protein, 12 aa, synthetic construct
SLSALLSSDIFN

SEQ ID NO: 38 - protein, 12 aa, synthetic construct
SLPRFKIIGGFN

SEQ ID NO: 39 - protein, 12 aa, synthetic construct
SLLGIAVPGNFN

SEQ ID NO: 40 - protein, 12 aa, synthetic construct
FFKNIVTPRTPP

SEQ ID NO: 41 - protein, 22 aa, synthetic construct
VKQTLNFDLLKLAGDVESNPGP

SEQ ID NO: 42 - protein, 36 aa, synthetic construct
MDAMKRGLCCVLLLCGAVFVSPSQEIHARFRRGARS

SEQ ID NO: 43 - protein, 7 aa, synthetic construct
ENLYFQG

SEQ ID NO: 44-45 - skipped (2 entries)

SEQ ID NO: 46 - protein, 746 aa, synthetic construct
  VARIANT 248: note = X can be any naturally occurring amino acid
  VARIANT 262: note = X can be any naturally occurring amino acid
  VARIANT 270: note = X can be any naturally occurring amino acid
  VARIANT 275: note = X can be any naturally occurring amino acid
  VARIANT 399: note = X can be any naturally occurring amino acid
  VARIANT 408: note = X can be any naturally occurring amino acid
  VARIANT 524: note = X can be any naturally occurring amino acid
  VARIANT 580: note = X can be any naturally occurring amino acid
  VARIANT 641: note = X can be any naturally occurring amino acid
  VARIANT 709: note = X can be any naturally occurring amino acid
  VARIANT 733: note = X can be any naturally occurring amino acid
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADTILHSPGCVPCVREGNASRCWVALTPTVAXRDGSLPTTQLRRHXDLLVGSAXLCSAXYVGDLCGSVFLVGQLFTFSPRRHWTTQDCNCSIYPGHITGHRMAWDMMMNWSPTTALVVAQLLRIPQAILDMIAGAHWGVLAGIAYFSMVGNWAKVLVVLLLFAGVDAETHVTGGSAARATSGXASLFSPGAXQNIQLINTNGSWHINRTALNCNDSLDTGWVAGLFYYHKFNSSGCPERMASCRPLADFDQGWGPISYANGSGPDQRPYCWHYPPKPCGIVPAQQVCGPVYCFTPSPVVVGTTDRLGXPTYNWGENETDVLVLNNTRPPLGNWFGCTWMNSTGFTKVCGAPPCNIGGVGNNTLXCPTDCFRKHPEATYSRCGSGPWLTPRCLVDYPYRLWHYPCTVNYTIFKVRMYVGGVEHRLXAACNWTRGERCDLDDRDRSELSPLLLSTTQWQVLPCSFTTLPALSTGLIHLHQNIVDVQYLYGVGSSXVSWAIKWEYVILLFLLLADARICXCLWMMLLISQAEA

SEQ ID NO: 47 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSTGLYHVTNDCPNSSIVYEAADAILHTPGCVPCVREGNTSRCWVAMTPTVATRDGKLPTTQLRRHIDLLVGSATLCSALYVGDLCGSIFLVGQMFTFSPRRHWTTQDCNCSLYPGHITGHRMAWDMMMNWSPTAALITAQLLRIPQAILDMIAGAHWGVLAGIAYFSMVGNWAKVLVVLLLFAGVDAQTHVTGGRAAHITAGLTSLFSPGPSQKLQLVNTNGSWHINSTALNCNDSLKTGWIAGLLYSYKFNSSGCPERLASCRRLTDFAQGWGPISHANGSGPDERPYCWHYPPRPCGIVPAKSVCGPVYCFTPSPVVVGTTDKSGAPTYNWGENDTDVFVLNNTRPPLGNWFGCTWMNSTGFTKVCGAPPCVIGGAGNNTLRCPTDCFRKHPDATYSRCGSGPWITPRCLVDYPYRLWHYPCTVNYSIFKIRMYLGGVEHRLEAACNWTRGERCDLEDRDRSELSPLLLSTTQWQVLPCSFTTLPALSTGLIHLHQNIVDVQYLYGVGSSVASWAIKWDYVVLLFLLLADARICSCLWMMLLISQAEA

SEQ ID NO: 48 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDAETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEA

SEQ ID NO: 49 - protein, 746 aa, Hepatitis C virus
MSTNPKPQRKTKRNTNRRPQDVKFPGGGQIVGGVYLLPRRGPRLGVRAVRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGIYHVTNDCPNSSIVYETADTILHSPGCVPCVREGNASKCWVALAPTVATRDGKLPTTQLRRHIDLLVGSATLCSALYVGDLCGSVFLVGQLFTFSPRRHWTTQDCNCSIYPGHITGHRMAWDMMMNWSPTTALVVAQLLRVPQAILDMIAGAHWGVLAGIAYFSMVGNWAKVLVVLLLFAGVDAETYTTGGSVAQAAFGLTSLFRPGPKQDIQLINTNGSWHINRTALNCNASLDTGWVAGLFYYHKFNSSGCPERMASCRSLADFDQGWGPISYANGSGPEHRPYCWHYPPKPCGIVPAQNVCGPVYCFTPSPVVVGTTNKLGVPTYSWGSNETDVLVLNNTRPPLGNWFGCTWMNSSGFTKVCGAPPCVIGGAGNRTLHCPTDCFRKHPEATYSRCGSGPWITPRCLVHYPYRLWHYPCTVNYTMFKVRMYVGGVEHRLEVACNWTRGERCDLDDRDRSELSPLLLSTTQWQVLPCSFTTLPALTTGLIHLHQNIVDVQYLYGVGSSIVSWAIKWEYVILLFLLLADARICSCLWMMLLISQAEA

SEQ ID NO: 50 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRSWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAFQVRNSSGLYHVTNDCPNSSIVYETADTILHSPGCVPCVREDNTSRCWVAVAPTVATRDGRLPTTQLRRHIDLLVGSATLCSALYVGDLCGSVFLVGQLFTFSPRRHWTTQDCNCSIYPGHITGHRMAWDMMMNWSPTTALVVAQLLRVPQAILDMIAGAHWGVLAGIAYFSMVGNWAKVLVVLLLFASVDAETYTSGGSVARATAGFAGIFNPGAKQDIQLINTNGSWHINRTALNCNASLDTGWVAGLFYYHKFNSSGCPERMASCKPLAHFAQGWGPISYANGSGPDHRPYCWHYPPKPCGIVPAQNVCGPVYCFTPSPVVVGTTNKLGAPTYNWGSNDTDVFILNNTRPPGGNWFGCTWMNSSGFTKVCGAPPCTIGGVGNNTLLCPTDCFRKHPEATYSRCGSGPWVTPRFLVHYPYRLWHYPCTVNYTLFKVRMYVGGVEHRLEVACNWTRGERCDLDDRDRSELSPLLLSTTQWQVLPCSFTTLPALTTGLIHLHQNIVDVQYLYGVGSSLVSWAIKWEYVILLFLLLADARICSCLWMMLLISQAEA

SEQ ID NO: 51 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADTILHSPGCVPCVREGNTSKCWVAVAPTVATRDGKLPTTQLRRHIDLLVGSATLCSALYVGDLCGSVFLVGQLFTFSPRRHWTTQDCNCSIYPGHITGHRMAWDMMMNWSPTAALVTAQLLRVPQAILDMIAGAHWGVLAGIAYFSMAGNWAKVLLVLLLFAGVDAETYTTGGSVARTTRGLASLLQVGPKQDIRLIHTNGSWHINRTALNCNASLDTGWLAGLLYYHKFNSSGCPERMASCRPLADFDQGWGPISYANGSGPEHRPYCWHYPPKPCGIVPAQTVCGPVYCFTPSPVVVGTTNKLGVPTYTWGSNDTDVFVLNNTRPPLGNWFGCTWMNSTGFTKVCGAPPCVIGGVGNNTLHCPTDCFRKHPEATYSRCGSGPWITPRCLVDYPYRLWHYPCTVNYTLFKVRMYVGGVEHRLQAACNWTRGERCDLDDRDRSELSPLLLSTTQWQVLPCSFTTLPALTTGLIHLHQNIVDVQYLYGVGSSIVSWAIKWEYVILLFLLLADARICSCLWMMLLISQAEA

SEQ ID NO: 52 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADTILHSPGCVPCVREDNASRCWVPVAPTVATRDGKLPTTQLRRHIDLLVGSATLCSALYVGDLCGSVFLVSQLFTFSPRRHWTTQDCNCSIYPGHITGHRMAWDMMMNWSPTVALVMAQLLRVPQAILDMIAGAHWGVLAGIAYFSMVGNWAKVLVVLLLFAGVDAQTYVTGGSAARGASGLANLFTPGAKQDIQLINTNGSWHINRTALNCNASLDTGWVAGLFYYHKFNSSGCPERMASCKPLADFDQGWGPIRHANGSGPEHRPYCWHYPPKPCGIVSAQTVCGPVYCFTPSPVVVGTTNRLGVPTYSWGTNDTDVFVLNNTRPPLGNWFGCTWMNSSGFTKVCGAPPCVIGGVGNNTLHCPTDCFRKHPEATYSRCGSGPWITPRCLVHYPYRLWHYPCTVNYTLFKVRMYVGGVEHRLEVACNWTRGERCDLDDRDRSELSPLLLSTTQWQVLPCSFTTLPALTTGLIHLHQNIVDVQYLYGVGSSIVSWAIKWEYVILLFLLLADARICSCLWMMLLISQAEA

SEQ ID NO: 53 - protein, 746 aa, Hepatitis C virus
MSTNPKPQRKTKRNTNRRPQDVKFPGGGQIVGGVYLLPRRGPRLGVRTTRKTSERSEPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETKDTILHSPGCVPCVREGNVSKCWVPVALTVATRDGNLPTTQLRRHIDLLVGSATLCSALYVGDLCGSVFLVGQLFTFSPRRHWTTQDCNCSMYPGHITGHRMAWDMMMNWSPTSALVVAQLLRVPQAILDMIAGAHWGVLAGLAYFSMVGNWAKVLVVLLLFASVDAGTHVTGGSAAHDVSALAGFFRRGAKQNIQLINTNGSWHVNRTALNCNASLDTGWVAGLLYYHRFNSSGCPERMASCRPLADFDQGWGPITNVDGGGSEYRPYCWHYPPKPCGIEPAQNVCGPVYCFTPSPVVVGTTDKVGVPTYNWGENDTDVFVLNNTRPPLGNWFGCTWMNSSGFVKVCGAPPCIIGGAGNKTLHCPTDCFRKHPDATYSRCGSGPWLTPRCLVDYPYRLWHYPCTVNYTLFKIRMYVGGVEHRLVAACNWTYGERCNLDDRDRSELSPLLLSTTQWQVLPCSFTTLPALTTGLIHLHQNIVDVQYLYGVGSSIVSWAIKWEYVILLFLLLADARICSCLWMMLLISQAEA

SEQ ID NO: 54 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADTILHSPGCVPCVREGNASKCWVAVAPTVATRDGTLPTTQLRRHIDLLVGGATLCSALYVGDLCGSVFLVGQLFTFSPRRHWTTQDCNCSIYPGHITGHRMAWDMMMNWSPTTALIVAQLLRVPQAILDMIAGAHWGVLAGIAYFSMVGNWAKVLVVLLLFAGVDAETHVTGGSAAQVTSRVAGFFNPGPKQNVQLINTNGSWHINRTALNCNASLDTGWVAGLFYHYNFNSSGCPERMASCRPLADFDQGWGPISYANGSGPEHRPYCWHYPPKPCGIVPAQNVCGPVYCFTPSPVVVGTTNRLGVPTYNWGSNDTDVFVLNNTRPPLGNWFGCTWMNSSGFTKVCGAPPCVIGGVGNNTLHCPTDCFRKHPEATYSRCGSGPWITPRCLVHYPYRLWHYPCTVNYTLFKVRMYVGGVEHRLEAACNWTRGERCDLDDRDRSELSPLLLSTTQWQVLPCSFTTLPALTTGLIHLHQNIVDVQYLYGVGSSIVSWAIKWEYVILLFLLLADARICSCLWMMLLISQAEA

SEQ ID NO: 55 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRSWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGIYHVTNDCPNSSIVYETADTILHSPGCVPCVREGNASKCWVALAPTVATRDGRLPTTQLRRHIDLLVGSATLCSALYVGDLCGSVFLVGQLFTFSPRRHWTTQDCNCSIYPGHITGHRMAWDMMMNWSPTTALVVAQLLRIPQAILDMIAGAHWGVLAGIAYFSMVGNWAKVLVVLLLFAGVDAHTRVTGGSAARATARLTTLFSPGAKQDIQLINTNGSWHINRTALNCNASLDTGWVAGLFYYHKFNSSGCPERMASCRPLADFDQGWGPISYANGSGPEHRPYCWHYPPKPCGIIPAKTVCGPVYCFTPSPVVVGTTDRSGAPTFNWGDNDTDVLVLNNTRPPLGNWFGCTWMNSSGYTKVCGAPPCIIGGVGNNTLHCPTDCFRKHPEATYSRCGSGPWITPRCLVHYPYRLWHYPCTVNYTLFKVRMYVGGVEHRLEAACNWTRGERCNLDDRDRSELSPLLLSTTQWQVLPCSFTTLPALTTGLIHLHQNIVDVQYLYGVGSSIVSWAIKWEYVILLFLLLADARVCSCLWMMLLISQVEA

SEQ ID NO: 56 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADTILHSPGCVPCVREGNASKCWVAVTPTVATRDGRLPATQLRRHIDLLVGSATLCSALYVGDLCGSIFLVGQLFTFSPRRHWTTQDCNCSIYPGHITGHRMAWDMMMNWSPTTALVVAQLLRVPQAILDMIAGAHWGVLAGIAYFSMVGNWAKVLVVLLLFAGVDAGTHVTGGSAAKDTSGFTSLFRIGARQNIQLINSNGSWHINRTALNCNESLDTGWVAGLLYYHKFNSSGCPERMASCRSLADFDQGWGPISYANGSGPEHRPYCWHYPPKPCGIVPAQSVCGPVYCFTPSPVVVGTTDKSGAPTYNWGCNETDVFVLNNTRPPLGNWFGCTWMNSSGFTKVCGAPPCVIGGVGNNTLHCPTDCFRKHPEATYSRCGSGPWITPRCLVHYAYRLWHYPCTVNYTLFKVRMYVGGVEHRLDVACNWTRGERCNLDDRDRSELSPLLLSTTQWQVLPCSFTTLPALSTGLIHLHQNIVDVQYLYGVGSSIVSWAIKWEYVILLFLLLADARICSCLWMMLLISQAEA

SEQ ID NO: 57 - protein, 746 aa, Hepatitis C virus; identical to SEQ ID NO: 56

SEQ ID NO: 58 - protein, 746 aa, Hepatitis C virus; identical to SEQ ID NO: 56

SEQ ID NO: 59 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADSILHSPGCVPCVREGNASKCWVAVAPTVATRDGKLPATQLRRHIDLLVGSATLCSALYVGDLCGSVFLVSQLFTFSPRRHWTTQDCNCSIYPGHITGHRMAWDMMMNWSPTTALVVSQLLRVPQAILDMIAGAHWGVLAGIAYFSMVGNWAKVLVVLLLFAGVDAETHTTGGSAAYATSGFVGLFRQGAKQNIQLINTNGSWHVNRTALNCNASLDTGWVAGLFYYHKFNSSGCPERLASCKPLANFDQGWGSISYTNGSGPEHRPYCWHYPPKPCGIVPAQNVCGPVYCFTPSPVVVGTTDRLGAPTFNWGENESDVFVLNNTRPPSGNWFGCTWMNSSGFTKVCGAPPCNIGGVGNNTLHCPTDCFRKHPEATYSRCGSGPWVTPRCLVHYPYRLWHYPCTLNYSLFKVRMYVGGIEHRLEVACNWTRGERCNLDDRDRSELSPLLLTTTQWQVLPCSFTTLPALTTGLIHLHQNVVDVQYLYGVGSSIVSWAIKWEYVILLFLLLADARICSCLWMMLLISQAEA

SEQ ID NO: 60 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADTILHSPGCVPCVREGNASKCWVAVAPTVATRDGRLPTTQLRRHIDLLVGSATLCSALYVGDLCGSVFLVGQLFTFSPRHHWTTQDCNCSIYPGHITGHRMAWDMMMNWSPTTALVVAQLLRVPQAILDMIAGAHWGVLAGIAYFSMVGNWAKVLVVLLLFAGVDATTHTTGGAVAHNTRMFTSIFSLGPRQEIQLVNTNGSWHINRTALNCNASLETGWIAGLLYANRFNSSGCPERMASCKPLADFDQGWGPISYANGSGPEHRPYCWHYPPKPCGIVPAQNVCGPVYCFTPSPVVVGTTDRLGTPTYDWGSNDTDVFVLNNTRPPAGNWFGCTWMNSSGYTKVCGAPPCVIGGVSNNTLHCPTDCFRKHPEATYSRCGSGPWITPRCLVHYAYRLWHYPCTVNYTLFKVRMYVGGVEHRLEVACNWTRGERCNLDDRDRSELSPLLLSTTQWQVLPCSFTTLPALTTGLIHLHQNIVDVQYLYGVGSSIVSWAVKWEYVILLFLLLADARICSCLWMMLLISQAEA

SEQ ID NO: 61 - protein, 746 aa, Hepatitis C virus
MSTNPKPQRKTKRNTNRRPQDVKFPGGGQIVGGVYLLPRRGPRLGVRATRKTSERSQPRGRRQPIPKARQPEGRAWAQPGYPWPLYGNEGLGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAYEVRNVSGVYHVTNDCSNASIVYEAADMIMHTPGCVPCVRENNSSRCWVALTPTLAARNASVPTTTIRRHVDLLVGAAALCSAMYVGDLCGSVFLVAQLFTFSPRRHETVQDCNCSIYPGHVTGHRMAWDMMMNWSPTAALVVSQLLRIPQAVVDMVAGAHWGVLAGLAYYSMVGNWAKVLIVMLLFAGVDGGTYVTGGTMAKNTLGITSLFSPGSSQKIQLVNTNGSWHINRTALNCNDSLNTGFLAALFYVHKFNSSGCPERMASCSPIDAFAQGWGPITYNESHSSDQRPYCWHYAPRPCGIVPAAQVCGPVYCFTPSPVVVGTTDRFGVPTYSWGENETDVLLLNNTRPPQGNWFGCTWMNSTGFTKTCGGPPCNIGGIGNKTLTCPTDCFRKHPEATYTKCGSGPWLTPRCLVHYPYRLWHYPCTVNFTIFKVRMYVGGVEHRLEAACNWTRGERCNLEDRDRSELSPLLLSTTEWQVLPCSFTTLPALSTGLIHLHQNVVDVQYLYGIGSAVVSFAIKWEYVLLLFLLLADARVCACLWMMLLIAQAEA

SEQ ID NO: 62 - protein, 746 aa, Hepatitis C virus
MSTNPKPQRKTKRNTNRRPQDVKFPGGGQIVGGVYLLPRRGPRLGVRATRKTSERSQPRGRRQPIPKARQPEGRAWAQPGYPWPLYGNEGMGWAGWLLSPRGSRPSWGPTDPRRRSRNLGKVIDTLTCGFADLMGYIPLVGAPLGGVARALAHGVRVVEDGVNYATGNLPGCSFSIFLLALLSCLTIPASAYEVRNVSGIYHVTNDCSNSSIVYEAADMIMHTPGCVPCVREGNSSRCWVALTPTLAARNASVPTTAIRRHVDLLVGAAAFCSAMYVGDLCGSVFLVSQLFTFSPRRHETIQDCNCSIYPGHVSGHRMAWDMMMNWSPTTALVVSQLLRIPQAIVDMVAGAHWGVLAGLAYYSMVGNWAKVLIVMLLFAGVDGETRVTGGQIARNAYSLTTLFSSGSAQNIQLINTNGSWHINRTALNCNDSLNTGFLAALFYTHKFNASGCPERLASCRPIDKFDQGWGPITYAEQGGQDQRPYCWHYAPKPCGIVSASKVCGPVYCFTPSPVVVGTTDRFGVPTYSWGENETDVLLLNNTRPPQGNWFGCTWMNGTGFTKTCGGPPCNIGGGGNNTLTCPTDCFRKHPAATYTKCGSGPWLTPRCLVDYPYRLWHYPCTANFTIFKVRMYVGGVEHRLDAACNWTRGERCNLEDRDRLELSPLLLSTTEWQVLPCSFTTLPALSTGLIHLHQNIVDVQYLYGIGSAVVSFAIKWDYIVILFLLLADARVCACLWMMLLIAQAEA

SEQ ID NO: 63 - protein, 747 aa, Hepatitis C virus
MSTNPKPQRKTKRNTNRRPQDVKFPGGGQIVGGVYLLPRRGPRLGVRAIRKTSERSQPRGRRQPIPKARRPEGRAWAQPGYPWPLYGNEGMGWAGWLLSPRGSRPSWGPTDPRRRSRNLGKVIDTLTCGLADLMGYIPLVGGPLGGAARALAHGVRVLEDGVNYATGNLPGCSFSIFLLALLSCLTVPASAHEVRNASGVYHVTNDCSNSSIVFEAADLIMHTPGCVPCVREGNSSRCWVALTPTLAARNATIPTTTIRHHVDLLVGAAALCSAMYVGDLCGSVFLVSQLFTFSPRRHATLQDCNCSIYPGHASGHRMAWDMMMNWSPTTALVVSQLLRIPQAVIDMVAGAHWGVLAGLAYYSMAGNWAKVLIVMLLFAGVDGHTLTTGGHAARLTSGFAGLFTPGPSQRIQLINTNGSWHINRTALNCNDSLQTGFLAALFYAHRFNSSGCPERMASCRSIDKFDQGWGPITYAEPTKDPDQRPYCWHYPPQQCGIVPASQVCGPVYCFTPSPVVVGTTDRLGNPTYSWGENDTDVLLLNNTRPPQGNWFGCTWMNSTGFTKTCGAPPCNIGGVGNNTLTCPTDCFRKHPEATYSKCGSGPWLTPRCMVDYPYRLWHYPCTVNFSIFKVRMYVGGVEHRLNAACNWTRGERCNLDDRDRSELSPLLLSTTEWQVLPCSFTTLPALSTGLIHLHQNIVDVQYLYGIGSAVVSFAIKWEYVVLLFLLLADARVCACLWMMLLIAQAEA

SEQ ID NO: 64 - protein, 746 aa, Hepatitis C virus
MSTNPKPQRKTKRNTNRRPQDVKFPGGGQIVGGVYLLPRRGPRLGVRATRKTSERSQPRGRRQPIPKARHPEGRTWAQPGYPWPLYGNEGLGWAGWLLSPRGSRPSWGPTDPRRRSRNLGKVIDTLTCGFADLMGYIPLVGAPLGGVARALAHGVRVLEDGVNYATGNLPGCSFSIFLLALLSCLTIPASAYEVRNVSGVYHVTNDCSNSSIVYEAADVIMHTPGCVPCVQDGNTSRCWVALTPTLAARNASVPVTAIRRHVDLLVGTAAFCSAMYVGDLCGSVFPVSQLFTFSPRRHQTVQDCNCSIYPGHISGHRMAWDMMMNWSPTAALVVSQLLRIPQAIVDMVAGAHWGVLAGLAYYSMVGNWAKVMIVLLLFAGVDGTTHTTGGAAARATQGFTSFFSLGPSQKIQLINTNGSWHINRTALNCNDSLQTGFLAALFYTYRFNASGCPERMASCRPIDKFDQGWGPITYAEPDSSDQRPYCWHYAPRPCGIVPASQVCGPVYCFTPSPVVVGTTDRFGVPTYTWGENETDVLLLNNTRPPLGNWFGCTWMNSTGFTKTCGGPPCNIGGAGNTTLTCPTDCFRKHPEATYTKCGSGPWLTPRCLVDYPYRLWHYPCAVNFTIFKVRMYVGGVEHRLNAACNWTRGERCDLEDRDRSELSPLLLSTTEWQVLPCSFTTLPALSTGLIHLHQNIVDVQYLYGIGSAVIPFAIKWEYVLLLFLLLADARVCACLWMMLLIAQAEA

SEQ ID NO: 65 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGMGWAGWLLSPRGSRPNWGPTDPRRRSRNLGKVIDTLTCGFADLMGYIPLVGAPLGGVARALAHGVRVLEDGVNYATGNLPGCSFSIFLLALLSCLTTPASAYEVRNVSGIYHVTNDCSNSSIVYEAADIIMHTPGCVPCVREKNISRCWVALTPTLAARNISVPTATIRRHVDLLVGTAAFCSAMYVGDLCGSVFLVSQLFTFSPRWHETVQDCNCSLYPGHVSGHRMAWDMMMNWSPTAALVVSQLLRIPQAVVDMVAGAHWGVLAGLAYYSMIGNWAKVLIVMLLFAGADGTTHVTGGVQAHGAYGLASLFNVGPHQKIQLVNTNGSWHINRTALNCNDTLQTGFLAALFYKHRFNASGCPERMASCRPIDKFAQGWGPITYAEPDRLDQRPYCWHYPPRPCGIVPALEVCGPVYCFTPSPVVVGTTDRFGVPTYSWGENETDVLLLNNTRPPQGNWFGCTWMNSTGYTKTCGGPPCNIGGVGNNTLTCPTDCFRKHPEATYTKCGSGPWLTPRCLVHYPYRLWHYPCTVNFTIFKVRMYVGGIEHRLDAACNWTRGERCDLEDRDRSELSPLLLSTTEWQILPCSFTTLPALSTGLIHLHRNIVDVQYLYGIGSAVVSFAIKWEYVLLLFLLLADARVCACLWMMLLIAQAEA

SEQ ID NO: 66 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGLGWAGWLLSPRGSRPSWGPNDPRRRSRNLGKVIDTLTCGFADLMGYIPLVGAPLGGAARALAHGVRVLEDGVNYATGNLPGCSFSIFLLALLSCLTIPASAYEVRNVSGIYHVTNDCSNSSIVYEAADVIMHAPGCVPCVRENNSSRCWVALTPTLAARNASVPTTTLRRHVDLLVGTAAFCSAMYVGDLCGSVFLISQLFTFSPRRHETVQDCNCSIYPGHVSGHRMAWDMMMNWSPTAALVVSQLLRIPQAVMDMVAGAHWGVLAGLAYYSMVGNWAKVLIVMLLFAGVDGHTRVTGGVQGHVTSTLTSLFRPGASQKIQLVNTNGSWHINRTALNCNDSLKTGFLAALFYTHKFNASGCPERMASCRSIDKFDQGWGPITYAQPDNSDQRPYCWHYAPRQCGIVPASQVCGPVYCFTPSPVVVGTTDRFGAPTYNWGDNETDVLLLNNTRPPHGNWFGCTWMNSTGFTKTCGGPPCNIRGVGNNTLTCPTDCFRKHPDATYTKCGSGPWLTPRCLVDYPYRLWHYPCTVNFTIFKVRMYVGGVEHRLDAACNWTRGERCDLEDRDRAELSPLLLSTTEWQILPCSYTTLPALSTGLIHLHQNIVDIQYLYGIGSAVVSIAIKWEYVVLLFLLLADARVCACLWMMLLIAQAEA

SEQ ID NO: 67 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGDEGMGWAGWLLSPRGSRPNWGPTDPRRRSRNLGKVIDTLTCGFADLMGYIPLVGAPLGGVARALAHGVRVLEDGVNYATGNLPGCSFSIFLLALLSCLTTPASAYEVRNVSGIYHVTNDCSNSSIVYEAADIIMHTPGCVPCVREKNTSRCWVALTPTLAARNISVPTTTIRRHVDLLVGTAAFCSAMYVGDLCGSVFLVSQLFTFSPRRHETVQDCNCSLYPGHVSGHRMAWDMMMNWSPTAALVVSQLLRIPQAIVDMVAGAHWGVLAGLAYYSMVGNWAKVLIVMLLFAGADGTTHVTGGVQAHGAYGLASLFNVGPHQKIQLVNTNGSWHINRTALNCNDTLQTGFLAALFYKHRFNASGCPERMASCRPIDKFAQGWGPITYAEPDRLDQRPYCWHYPPRPCGIVPALEVCGPVYCFTPSPVVVGTTDRFGVPTYSWGENETDVLLLNNTRPPQGNWFGCTWMNNTGFTKTCGGPPCNIGGVGNNTLTCPTDCFRKHPEATYTKCGSGPWLTPRCLVDYPYRLWHYPCTVNFTVFKVRMYVGGIEHRLDAACNWTRGERCDLEDRDRSELSPLLLSTTEWQILPCSFTTLPALSTGLIHLHRNIVDVQYLYGIGSAVVSFAIKWEYILLLFLLLADARVCACLWMMLLIAQAEA

SEQ ID NO: 68 - protein, 746 aa, Hepatitis C virus
MSTNPKPQRKTKRNTNRRPQDVKFPGGGQIVGGVYLLPRRGPRLGVRATRKTSERSQPRGRRQPIPKARQPEGRAWAQPGYPWPLYGNEGLGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAYEVRNVSGVYHVTNDCSNSSIVYEAADMIMHTPGCVPCVREDNSSRCWVALTPTLAARNSSVPTTTIRRHVDLLVGAAAFCSAMYVGDLCGSVFLISQLFTFSPRRYETVQDCNCSLYPGHVSGHRMAWDMMMNWSPTTALVVSQLLRIPQAVVDMVAGAHWGVLAGLAYYSMVGNWAKVLIVMLLFAGVDGNTRVSGGEAAKNTMGFASLFVSGPSQKIQLINTNGSWHINRTALNCDDSLHTGFLAALFYAHKFNSSGCSGRMASCRPIDEFAQGWGPITHGVPDNLDQRPYCWHYAPRPCGIVPASQVCGPVYCFTPSPVVVGTTDRFGAPTYSWGENETDVLLLNNTRPPQGNWFGCTWMNSTGFTKTCGGPPCNIGGVGNNTLTCPTDCFRKHPEATYTKCGSGPWLTPRCMVDYPYRLWHYPCTVNFTIFKVRMYVGGVEHRLDAACNWTRGERCNVEDRDRSELSPLLLSTTEWQILPCSFTTLPALSTGLIHLHQNIVDVQYLYGVGSVVVSVVIRWEYVVLLFLLLADARVCACLWMMLLIAQAEA

SEQ ID NO: 69 - protein, 746 aa, Hepatitis C virus
MSTNPKPQRKTKRNTNRRPQDVKFPGGGQIVGGVYLLPRRGPRLGVRATRKTSERSQPRGRRQPIPKARQPEGRAWAQPGYPWPLYGNEGMGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAYEVRNVSGMYHVTNDCSNSSIVYEAADMILHAPGCVPCVRENNSSRCWVALTPTLAARNASVPTTAIRRHVDLLVGAAAFCSAMYVGDLCGSVLLVSQIFTFSPRRHETMQDCNCSIYPGHITGHRMAWDMMMNWSPTTALVVSQLLRIPQAIVDMVAGAHWGVLAGLAYYSMVGNWAKVLIVMLLFAGVDGGTYVTGGEAGRRTSGFASIFTPGASQNIQLINTNGSWHINRTALNCNDSLHTGFIAALFYHHKFNASGCPERMASCRPIGEFAQGWGPISYTEPPSSDQRPYCWHYPPRPCGIVPASQVCGPVYCFTPSPVVVGTTDRLGAPTYNWGDNDTDVLLLNNTRPPQGNWFGCTWMNGTGFTKTCGGPPCNIGGVGNNTLTCPTDCFRKHPEATYTKCGSGPWLTPRCIVDYPYRLWHYPCTVNFTITKIRMYVGGVEHRLTAACNWTRGERCDLEDRDRSELSPLLLSTTEWQIMPCSFTTLPALSTGLIHLHQNIVDIQYLYGIGSAAVSFAIRWEYVLLLFLLLADARVCACLWMMLLIAQAEA

SEQ ID NO: 70 - protein, 746 aa, Hepatitis C virus; identical to SEQ ID NO: 69

SEQ ID NO: 71 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRAWAQPGYPWPLYGNEGLGWAGWLLSPRGSRPSWGPTDPRRRSRNLGKVIDTLTCGFADLMGYIPLVGAPLGGTARALAHGVRVLEDGVNYATGNLPGCSFSIFLLALLSCLTIPASAYEVRNVSGVYHVTNDCSNSSIVYETADMIMHTPGCVPCVREDNFTRCWVALTPTLAARNGSVPTTAIRRHVDLLVGAAAFCSAMYVGDLCGSVFLVSQLFTFSPRWHETVQECNCSIYPGHVTGHRMAWDMMMNWSPTTALVVSQLLRIPQAVVDMVAGAHWGVLAGLAYYSMVGNWAKVLIVTLLFAGVDGNTHTIGGKQAQATGGFVAWLARGPSQEIQLINTNGSWHINRTALNCNDSLKTGFIAALFYAHRFNSSGCPERMASCRPIDKFAQGWGPITYAKPDSLDQRPYCWHYAPQPCGIVPASEVCGPVYCFTPSPVVVGTTDRSGVPTYRWGENETDVLLLNNTRPPQGNWFGCTWMNATGFTKTCGGPPCKIGGLGNNTLTCPTDCFRKHPEATYTKCGSGPWLTPRCIVDYPYRLWHYPCTVNFTIFKVRMYVGGIEHRLSAACNWTRGERCDLEDRDRSELSPLLLSTTEWQILPCSFTTLPALSTGLIHLHQNTVDVQYLYGVGSVLVSFAIKWEYILLFFLLLADARVCACLWMMLLIAQAEA

SEQ ID NO: 72 - protein, 746 aa, Hepatitis C virus
MSTNPKPQRKTKRNTNRRPQNVKFPGGGQIVGGVCLLPRRGPRVGVRATRKTSERSQPRGRRQPIPKARRPEGRSWAQPGYPWPLYGNEGCGWAGWLLSPRGSRPSRGPSDPRRRSRNLGKVIDTLTCGFADLMGYIPLVGAPLGGAARALAHGVRVLEDGVNYATGNLPGCSFSIFLLALLSCLTVPASAVEVRNSSGIYHVTNDCPNASVVYETDSLIIHLPGCVPCVREGNASRCWVSLSPTVAAKDPGVPVNEIRRHVDLIVGAAAFCSAMYVGDLCGSIFLVGQLFTLSPRRHWTTQDCNCSIYPGHVTGHRMAWDMMMNWSPTGALVVAQLLRIPQAVLDMIAGAHWGVLAGPAYYSMVGNWAKVLVVLLLFAGVDATTQVTGGTAGRNAYRLASLFSTGPSQNIQLINSNGSWHINRTALNCNDSLHTGWVAALFYSHKFNSSGRPERMASCRPLTAFDQGWGPITYGGKASNDQRPYCWHYAPRPCGIVPAKEVCGPVYCFTPSPVVVGTTDKYGVPTYTWGENETDVLLLNNSRPPIGNWFGCTWMNSTGFTKTCGAPACNVGGSETNTLSCPTDCFRRHPDATYAKCGSGPWLNPRCMVDYPYRLWHYPCTVNYTIFKIRMFVGGIEHRLTAACNWTRGERCDLDDRDRAELSPLLLSTTQWQVLPCSFTTLPALSTGLIHLHQNIVDVQYLYGLSSVVTSWAIRWEYVVLLFLLLADARICACLWMMLLISQVEA

SEQ ID NO: 73 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTYGFADLMGYIPLVGAPLGGAARALAHGVRVLEDGVNYATGNLPGCSFSIFLLALLSCLTVPASAVGVRNSSGVYHVTNDCPNASVVYETDSLIIHLPGCVPCVREGNGSRCWVSLSPTVAAKDPGVPVNEIRRHVDLIAGAAAFCSAMYVGHLCGSIFLVGQLFTLSPRRHWTTQDCNCSIYPGHVTGHRMAWDMMMNWSPTTALVVAQLLRIPQAILDMIAGAHWGVLAGIAYFSMVGNWTKVLVVLLLFAGVDATTIVSGGSAGRSTAGLVGLFSPGARQNIQLINTNGSWHINRTALNCNDTLQTGWVAGLFYTNKFNSSGCPERLASCRPLADFDQGWGPISYTNGSGPDQRPYCWHYPPKPCGIVPAESVCGPVYCFTPSPVVVGTTDRSGAPTYNWGENETDVFVLNNTRPRLGNWFGGTWMNSTGFTKVCGAPPCAIGGVGNNTLYCPTDCFRKHPEATYSRCGSGPWITPRCLIHYPYRLWHYPCTINYTIFKIRMFVGGVEHRLDAACNWTRGERCDLDDRDRAELSPLLLSTTQWQVLPCSFTTLPALSTGLIHLHQNIVDVQYLYGLSSAVTSWVIKWEYVVLLFLLLADARICACLWMMLLISQVEA

SEQ ID NO: 74 - protein, 746 aa, Hepatitis C virus
MSTNPKPQRKTKRNTNRRPQDVKFPGGGQIVGGVYLLPRRGPRVGVRATRKTSERSQPRGRRQPIPKARRPEGRSWAQPGYPWPLYGNEGCGWAGWLLSPRGSRPSWGPSDPRRRSRNLGKVIDTLTCGFADLMGYIPLVGAPLGGAARALAHGVRVLEDGVNYATGNLPGCSFSIFLLALLSCLTVPASAVGVRNSSGVYHVTNDCPNASVVYETENLIMHLPGCVPYVREGNASRCWVSLSPTVAARDSRVPVSEVRRRVDSIVGAAAFCSAMYVGDLCGSIFLVGQIFTFSPRHHWTTQDCNCSIYPGHVTGHRMAWDMMMNWSPTGALVVAQLLRIPQAIVDMIAGAHWGVLAGLAYYSMVGNWAKVVVVLLLFAGVDAETRVTGGAAGHTAFGFASFLAPGAKQKIQLINTNGSWHINRTALNCNESLDTGWLAGLLYYHKFNSSGCPERMASCQPLTAFDQGWGPITHEGNASDDQRPYCWHYALRPCGIVPAKKVCGPVYCFTPSPVVVGTTDRAGVPTYRWGANETDVLLLNNSRPPMGNWFGCTWMNSSGFTKTCGAPACNIGGSGNNTLLCPTDCFRKHPDATYSRCGSGPWLTPRCLVDYPYRLWHYPCTVNYTIFKIRMFVGGVEHRLDAACNWTRGERCDLDDRDRAELSPLLLSTTQWQVLPCSFTTLPALSTGLIHLHQNIVDVQYLYGLSSAVTSWVIKWEYVVLLFLLLADARICACLWMMLLISQVEA

SEQ ID NO: 75 - protein, 749 aa, synthetic construct
  VARIANT 233: note = X can be any naturally occurring amino acid
  VARIANT 249..250: note = X can be any naturally occurring amino acid
  VARIANT 290: note = X can be any naturally occurring amino acid
  VARIANT 378: note = X can be any naturally occurring amino acid
  VARIANT 392: note = X can be any naturally occurring amino acid
  VARIANT 399: note = X can be any naturally occurring amino acid
  VARIANT 445: note = X can be any naturally occurring amino acid
  VARIANT 495: note = X can be any naturally occurring amino acid
  VARIANT 627: note = X can be any naturally occurring amino acid
  VARIANT 663: note = X can be any naturally occurring amino acid
  VARIANT 682: note = X can be any naturally occurring amino acid
MSTNPKPQRKTKRNTNRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPTDPRHRSRNVGKVIDTLTCGFADLMGYIPVVGAPLGGVARALAHGVRVLEDGVNYATGNLPGCSFSIFLLALLSCITVPVSAVEVKNISTSYMVTNDCSNDSITWQLQAAVLHVPGCVPCENDXNTSRCWIPVSPNVAVXXPGALTQGLRTHIDMVVMSATLCSALYVGDLCGGVMLAAQXFIVSPQRHWFVQECNCSIYPGTITGHRMAWDMMMNWSPTATMILAYAMRVPEVIIDIIFGAHWGVMFGLAYFSMQGAWAKVIVILLLXAGVDARTHTVGGSXGRTTSGXAGLFSSGPKQNIQLINTNGSWHINRTALNCNDSLQTGFIASLFYTXNFNSSGCPERLSACRGIEAFRIGWGTLQYEDNVTNPEDMRPYCWHYPPKXCGIVPARSVCGPVYCFTPSPVVVGTTDRLGVPTYTWGENETDVFLLNSTRPPRGSWFGCTWMNSTGFTKTCGAPPCRIRADFNASTDLLCPTDCFRKHPDATYKCGSGPWLTPRCLVDYPYRLWHYPCTVNXTIFKIRMYVGGVEHRLTAACNFTRGDRCNLEDRDRXQLSPLLHSTTEWAILPCSXSDLPALSTGLLHLHQNIVDVQYMYGLSPALTKYIVRWEWVVLLFLLLADARVCACLWMLILLGQAEA

SEQ ID NO: 76 - protein, 750 aa, Hepatitis C virus
MSTNPKPQRKTKRNTNRRPEDVKFPGGGQIVGGVYLLPRRGPRLGVRTTRKTSERSQPRGRRQPIPKDRRSTGKAWGKPGRPWPLYGNEGLGWAGWLLSPRGSRPSWGPTDPRHRSRNVGKVIDTLTCGFADLMGYIPVVGAPLSGAARAVAHGVRVLEDGVNYATGNLPGFPFSIFLLALLSCITVPVSAAQVKNTSSSYMVTNDCSNDSITWQLEAAVLHVPGCVPCERVGNTSRCWVPVSPNMAVRQPGALTQGLRTHIDMVVMSATFCSALYVGDLCGGVMLAAQVFIVSPQYHWFVQECNCSIYPGTITGHRMAWDMMMNWSPTATMILAYVMRVPEVIIDIVSGAHWGVMFGLAYFSMQGAWAKVIVILLLAAGVDAGTTTVGGAVARSTNVIAGVFSHGPQQNIQLINTNGSWHINRTALNCNDSLNTGFLAALFYTNRFNSSGCPGRLSACRNIEAFRIGWGTLQYEDNVTNPEDMRPYCWHYPPKPCGVVPARSVCGPVYCFTPSPVVVGTTDRRGVPTYTWGENETDVFLLNSTRPPQGSWFGCTWMNSTGFTKTCGAPPCRTRADFNASTDLLCPTDCFRKHPDATYIKCGSGPWLTPKCLVHYPYRLWHYPCTVNFTIFKIRMYVGGVEHRLTAACNFTRGDRCDLEDRDRSQLSPLLHSTTEWAILPCTYSDLPALSTGLLHLHQNIVDVQYMYGLSPAITKYVVRWEWVVLLFLLLADARVCACLWMLILLGQAEA

SEQ ID NO: 77 - protein, 749 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPTDPRHRSRNVGKVIDTLTCGFADLMGYIPVVGAPLGGVARALAHGVRVLEDGVNYATGNLPGCSFSIFLLALLSCITTPVSAAQVKNTSDIYMVTNDCSNDSITWQLQAAALHVPGCVPCERVGNTSRCWIPVSPNVAVRQPGALTQGLRTHIDMVVMSATLCSALYVGDLCGGVMLAAQMFIISPQHHWFVQECNCSIYPGTITGHRMAWDMMMNWSPTTTMILAYVMRVPEVIIDIIGGAHWGVMFGLAYFSMQGAWAKVVVILLLAAGVDAHTRTGSSVGYATSGIVGLFTSGPKQNIQLINTNGSWHINRTALNCNDSLNTGFIVSLFYARNFNSTGCPERLSACRGIEGFRIGWGTLQYEDNVTNPEDMRPYCWHYPPKQCGIVPAGSVCGPVYCFTPSPVVVGTTDRLGVPTYTWGENETDVFLLNSTRPPVGSWFGCTWMNSTGFTKTCGAPPCRIRADFNASTDLLCPTDCFRKHPEATYIKCGSGPWLTPRCLVDYPYRLWHYPCTVNYSIFKIRMYVGGVEHRLTAACNFTRGDRCNLEDRDRSQLTPLLHSTTEWAILPCTYSDLPALSTGLLHLHQNIVDVQYMYGLSPALTKYVVRWEWVVLLFLLLADARVCACVWMLILLGQAEA

SEQ ID NO: 78 - protein, 750 aa, Hepatitis C virus
  VARIANT 458: note = X can be any naturally occurring amino acid
MSTNPKPQRKTKRNTNRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPTDPRHRSRNVGKVIDTLTCGFADLMGYIPVVGAPLGGVARALAHGVRVLEDGVNYATGNLPGCSFSIFLLALLSCVTVPVSAVQVKNTSETYMVTNDCSNDSITWQLQAAVLHVPGCVPCERTGNTSRCWIPVSPNVAVQQPGALTQGLRTHIDMVVMSATLCSALYVGDLCGGVVLAAQLFIVSPRRHWFVQECNCSIYPGAITGHRMAWDMMMNWSPTTTMVLAYAMRVPEVIIDIISGAHWGVMFGLAYFSMQGAWAKVAVILLLTAGVEARTHTTGSVAGRTTSGFAGIFTSGPKQNIQLINTNGSWHINRTALNCNDSLNTGFMAALFYTKNFNSSGCPERLSXCRNIEAFRIGWGTLQYEDDVTNPEDMRPYCWHYPPKQCGIFPAGSVCGPVYCFTPSPVVVGTTNKLGVPTYTWGENETDVFILNSTRPPRGSWFGCTWMNSTGFTKTCGAPPCRIRADFNASTDLLCPTDCFRKHPEATYIKCGSGPWLTPRCLVDYPYRLWHYPCTVNYSIFKIRMYVGGVEHRLTAACNFSRGDRCNLEDRDRSQLTPLLHSTTEWAILPCSYSDLPALSTGLLHLHQNIVDVQYMYGLTPALTKYVVRWEWVVLLFLLLADARVCACLWMLILLGQAEA

SEQ ID NO: 79 - protein, 750 aa, Hepatitis C virus
MSTNPKPQRKTKRNTSRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPTDPRHRSRNVGKVIDTLTCGFADLMGYIPVVGAPLGGVARALAHGVRVLEDGVNYATGNLPGCSFSIFLLALLSCITVPVSAVQVKNISDSYMVTNDCSNDSITWQLQAAVLHVPGCVPCEKMGNISRCWIPVSPNVAVQRPGALTQGLRAHIDMVVMSATLCSALYVGDLCGGVMLAAQMFIVSPQHHHFVQECNCSIYPGAITGHRMAWDMMMNWSPTATMILAYAMRVPEVIIDIISGAHWGVMFGLAYFSMQGAWAKVVVILLLTAGVDAHTRSIAGSVAHATSGLAGLFTSGAKQNIQLINTNGSWHINRTALNCNDSLNTGFIASLFYTYRFNSSGCPERLSACRGIQAFRIGWGTLRYEDNVTNPEDMRPYCWHYPPKQCGIVSARSVCGPVYCFTPSPVVVGTTDRLGVPTYTWGENETDVFILNSTRPPGGSWFGCTWMNSTGFTKTCGAPPCRIRADFNASMDLLCPTDCFRKHPDATYIKCGSGPWLTPRCLVDYPYRLWHYPCTINYTIFKIRMYVGGVEHRLTAACNFTRGDPCNLEDRDRSQLSPLLHSTTEWAILPCSYSDLPALSTGLLHLHQNIVDVQYMYGLSPALTKYIVRWEWVVLLFLLLADARVCACLWMLILLGQAEA

SEQ ID NO: 80 - protein, 751 aa, Hepatitis C virus
MSTNPKPQRKTQRNTNRRPQDVKFPGGGQIVGGVYLLPRRGPRLGVRTARKTSERSQPRGRRQPIPKDRRSTGKSWGRPGYPWPLYGNEGLGWAGWLLSPRGSRPSWGPTDPRHRSRNVGKVIDTLTCGFADLMGYIPVVGAPLGGVARALAHGVRVLEDGVNYATGNLPGCSFSIFLLALLSCITIPASAVEVKNTSTGYMVTNDCANSSITWQLHAAVLHVPGCVPCERVDNNTSRCWIPVSPNIAVQRPGALTQGLRSHIDIVVMSATLCSALYVGDLCGGVMLAAQMFVVSPEHHWFVQECNCSIYPGTITGHRMAWDMMMNWSPTATMILAYAMRVPEVIIDIIGGAHWGVMFGLAYFSMQGAWAKVVVILLLAAGVDAYTHTVGGAAASTANSIAGLLSRGPRQNLQLINSNGSWHINRTALNCHDSLQTGFITALFYARHFNSSGCPERLAACRNIEAFRVGWGALQYEDNVTNPEDMRPYCWHYPPKQCGIVPARSVCGPVYCFTPSPVVVGTTDKLGVPTYTWGENETDVFLLNSTRPPQGPWFGCTWMNSTGFTKTCGAPPCRTRADFNASTDLLCPTDCFRKHPDATYNKCGSGPWLTPRCLIDYPYRLWHYPCTVNYTTFKIRMYVGGVEHRLMAACNFTRGDSCDLSQRDRGQLSPLLHSTTEWAILPCSFSDLPALSTGLLHLHQNIVDVQYMYGLSPALTKYIVRWEWVVLLFLLLADARVCACIWMLILLGQAEA

SEQ ID NO: 81 - protein, 750 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPNDPRHRSRNVGKVIDTLTCGFADLMGYIPVVGAPLGGVARALAHGVRVLEDGVNFATGNLPGCSFSIFLLALLSCITTPVSAAEVKNISTGYMVTNDCTNDSITWQLQAAVLHVPGCVPCEKVGNTSRCWIPVSPNVAVQQPGALTQGLRTHIDMVVMSATLCSALYVGDLCGGVMLAAQMFIVSPQHHWFVQDCNCSIYPGTITGHRMAWDMMMNWSPTATMILAYAMRVPEVIIDIIGGAHWGVMFGLAYFSMQGAWAKVVVILLLAAGVDAQTHTVGGSTAHNARTLTGMFSLGARQKIQLINTNGSWHINRTALNCNDSLHTGFLASLFYTHSFNSSGCPERMSACRSIEAFRVGWGALQYEDNVTNPEDMRPYCWHYPPRQCGVVSASSVCGPVYCFTPSPVVVGTTDRLGAPTYTWGENETDVFLLNSTRPPQGSWFGCTWMNSTGYTKTCGAPPCRIRADFNASMDLLCPTDCFRKHPDTTYIKCGSGPWLTPRCLIDYPYRLWHYPCTVNYTIFKIRMYVGGVEHRLTAACNFTRGDRCNLEDRDRSQLSPLLHSTTEWAILPCTYSDLPALSTGLLHLHQNIVDVQFMYGLSPALTKYIVRWEWVVLLFLLLADARVCACLWMLILLGQAEA

SEQ ID NO: 82 - protein, 750 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPNDPRHRSRNVGKVIDTLTCGFADLMGYIPVVGAPLGGVARALAHGVRVLEDGVNFATGNLPGCSFSIFLLALLSCITTPVSAAEVKNISTGYMVTNDCTNDSITWQLQAAVLHVPGCVPCEKVGNASQCWIPVSPNVAVQRPGALTQGLRTHIDMVVMSATLCSALYVGDLCGGVMLAAQMFIVSPQHHWFVQDCNCSIYPGTITGHRMAWDMMMNWSPTATMILAYAMRVPEVIIDIISGAHWGVMFGLAYFSMQGAWAKVVVILLLAAGVDARTHTVGGSAAQTTGRLTSLFDMGPRQKIQLVNTNGSWHINRTALNCNDSLHTGFIASLFYTHSFNSSGCPERMSACRSIEAFRVGWGALQYEDNVTNPEDMRPYCWHYPPRQCGVVSAKTVCGPVYCFTPSPVVVGTTDRLGAPTYTWGENETDVFLLNSTRPPLGSWFGCTWMNSSGYTKTCGAPPCRTRADFNASTDLLCPTDCFRKHPDTTYLKCGSGPWLTPRCLIDYPYRLWHYPCTVNYTIFKIRMYVGGVEHRLTAACNFTRGDRCNLEDRDRSQLSPLLHSTTEWAILPCSYSDLPALSTGLLHLHQNIVDVQFMYGLSPALTKYIVRWEWVILLFLLLADARVCACLWMLILLGQAEA

SEQ ID NO: 83 - protein, 750 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGCGWAGWLLSPRGSRPTWGPTDPRHRSRNLGRVIDTITCGFADLMGYIPVVGAPVGGVARALAHGVRVLEDGINYATGNLPGCSFSIFLLALLSCVTVPVSAVEVRNISSSYYATNDCSNNSITWQLTDAVLHLPGCVPCENDNGTLHCWIQVTPNVAVKHRGALTRSLRTHVDMIVMAATACSALYVGDVCGAVMILSQAFMVSPQRHNFTQECNCSIYQGHITGHRMAWDMMLSWSPTLTMILAYAARVPELVLEIIFGGHWGVVFGLAYFSMQGAWAKVIAILLLVAGVDATTYSSGQEAGRTVAGFAGLFTTGAKQNLYLINTNGSWHINRTALNCNDSLQTGFLASLFYTHKFNSSGCPERLSSCRGLDDFRIGWGTLEYETNVTNDGDMRPYCWHYPPRPCGIVPARTVCGPVYCFTPSPVVVGTTDKQGVPTYTWGENETDVFLLNSTRPPRGAWFGCTWMNGTGFTKTCGAPPCRIRKDYNSTIDLLCPTDCFRKHPDATYLKCGAGPWLTPRCLVDYPYRLWHYPCTVNFTIFKARMYVGGVEHRFSAACNFTRGDRCRLEDRDRGQQSPLLHSTTEWAVLPCSFSDLPALSTGLLHLHQNIVDVQYLYGLSPALTRYIVKWEWVILLFLLLADARICACLWMLIILGQAEA

SEQ ID NO: 84 - protein, 750 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGCGWAGWLLSPRGSRPHWGPTDPRHRSRNLGKVIDTITCGFADLMGYIPVIGAPVGGVARALAHGVRVLEDGVNYATGNLPGCSFSIFLLALLSCVTVPVSAVEVRNISSSYYATNDCSNNSITWQLENAVLHLPGCVPCENDNGTLRCWTQVTPNVAVKHRGALTQNLRTHVDVIVVAATVCSALYVGDVCGAVMIASQALIVSPARHNFTQECNCSIYQGRITGHHMAWDMMLNWSPTITMILAYAARIPELVLEVIFGGHWGVMFGLAYFSMQGAWAKVIVILLLVAGVDARHHTTGLQAGKTLARVTSLFSIGAKQNIQLINTNGSWHINRTALNCNDSLQTGFIASLFYVNNINSSGCPERMSSCRELDDFRIGWGTLEYETNVTNDEDMRPYCWHYPPKPCGIVPARTVCGPVYCFTPSPIVVGTTDKQGVPTYSWGENETDVFLLNSTRPPRGSWFGCTWMNGTGFTKTCGAPPCRIRRDYNSTLDLLCPTDCFRKHPDTTYLKCGSGPWLTPKCLVEYPYRLWHYPCTVNFTIFKVRMYVGAVEHRFSAACNFTRGDRCRLEDRDRGQQSPLLHSTTEWAVLPCSFSDLPALSTGLLHLHQNIVDVQYLYGLSPAITRYIVKWEWVVLLFLLLADARVCACLWMLIILGQAEA

SEQ ID NO: 85 - protein, 750 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGCGWAGWLLSPRGSRPTWGPTDPRHRSRNLGRVIDTITCGFADLMGYIPVVGAPVGGVARALAHGVRVLEDGINYATGNLPGCSFSIFLLALLSCCTVPVSAVEVRNISTSYYATNDCSNTSITWQLTNAVLHLPGCVPCENDNGTLRCWIQVTPNVAVKHRGALTHNLRTHVDVIVMAATVCSALYVGDICGAVMIVSQAFIISPERHNFTQECNCSMYQGHITGHRMAWDMMLNWSPTLTMILAYAARVPELVLEVIFGGHWGVVFGLAYFSMQGAWAKVIAILLLVAGVDANTYSSGVTVGHTTSTFANIFSVGPSQKINLINTNGSWHINRTALNCNDSLQTGFLASLFYVRNFNSSGCRERLSSCRRLDDFRIGWGTLEYETNVTNDEDMRPYCWHYPPKPCGIVSARTVCGPVYCFTPSPVVVGTTDRQGVPTYSWGENETDVFLLNSTRPPRGAWFGCTWMNGTGFTKTCGAPPCRIRRDYNSTLDLLCPTDCFRKHPDTTYLKCGAGPWLTPKCLVDYPYRLWHYPCTVNFTIFKVRMYVGGVEHRLSAACNFTRGDRCGLEDRDRGQQSPLLHSTTEWAVLPCSFSDLPALSTGLLHLHQNIVDVQYLYGLSPAITRYIVKWEWVVLLFLLLADARVCACLWMLIILGQAEA

SEQ ID NO: 86 - protein, 750 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGCGWAGWLLSPRGSRPTWGPSDPRHRSRNLGRVIDTITCGFADLMGYIPVVGAPVGGVARALAHGVRVLEDGINYATRNLPGCSFSIFLLALLSCVTVPVSSVEIRNISTSYYATNDCSNNSITWQLTNAVLHLPGCVPCENDNGTLRCWIQVTPNVAVKHRGALTHNLRAHVDVIVMAATVCSALYVGDVCGAVMIVSQALIVSPERHNFTQECNCSIYQGHITGQRMAWDMMLNWSPTLTMILAYAARVPELVLEIVFGGHWGVVFGLAYFSMQGAWAKVIAILLLVAGVDATTYSTGATVGRTVGSFAGLFKLGAQQNVQLINTNGSWHINRTALNCNDSLHTGFMAALFYANKFNSSGCPERLSSCRGLDDFRIGWGTLEYETNVTNVEDMRPYCWHYPPKPCGIVPAQSVCGPVYCFTPSPVVVGTTDRQGVPTYNWGDNETDVFLLNSTRPPRGAWFGCTWMNGTGFTKTCGAPPCRIRKDFNSTLDLLCPTDCFRKHPDATYVKCGAGPWLTPRCLIDYPYRLWHYPCTVNFTIFKVRMYVGGVEHRFSAACNFTRGDRCRLEDRDRGQQSPLLHSTTEWAVLPCSFSDLPALSTGLLHLHQNIVDVQYLYGLSPAVTKYIVKWEWVVLLFLLLADARICACLWMLIILGQAEA

SEQ ID NO: 87 - protein, 750 aa, Hepatitis C virus
  VARIANT 203: note = X can be any naturally occurring amino acid
  VARIANT 295: note = X can be any naturally occurring amino acid
  VARIANT 343: note = X can be any naturally occurring amino acid
  VARIANT 401: note = X can be any naturally occurring amino acid
  VARIANT 426: note = X can be any naturally occurring amino acid
  VARIANT 626: note = X can be any naturally occurring amino acid
MSTDPKPQRKTKRNTNRRPQDVKFPGGGQIVGGVYLLPRRGPRLGVRAARKTSERSQPRGRRQPIPKDRRSPGKSWGKPGYPWPLYGNEGCGWAGWLLSPRGSRPTWGPTDPRHRSRNLGKVIDTITCGFADLMGYIPVVGAPVGGVARALAHGVRVLEDGINYATGNLPGCSFSIFLLALLSCLTVPASAVEVRNISSGYYXTNDCSNSSITWQLTNAVLHLPGCVPCENDNGTLRCWIQVTPNVAVKYRGALTHNLRTHVDMIVMAATVCSALYVGDVCGAVMIVSQAFIMSXERHNFTQECNCSIYQGHITGHRMAWDMMLGWSPTLTMILAYAARVPEXVLEVVFGGHWGVVFGLAYFSMQGAWAKVIAILLLVAGVDAGTYSSGATIGQGTRGLVXLFSAGPSQKISLINTNGSWHINRTXLNCNDSLQTGFIASLFYAKSFNSSGCPERLSSCRGLDDFRIGWGTLEYENNVTNDEDMRPYCWHYPPKPCGIVPARTVCGPVYCFTPSPVVVGTTDKQGVPTYSWGENETDVFLLNSTRPPQGAWFGCTWMNGTGFTKTCGAPPCRIRRDHTSTLDLLCPTDCFRKHPDTTYLKCGAGPWLTPKCLVDYPYRLWHYPCTXNFTIFKVRMYVGGVEHRFSAACNFTRGDRCRLEDRDRGQQSPLLHSTTEWAVLPCSFSDLPALSTGLLHLHQNIVDVQYLYGLSPAITKYIVKWEWVILLFLLLADARVCACLWMLIILGQAEA

SEQ ID NO: 88 - protein, 752 aa, synthetic construct
  VARIANT 431: note = X can be any naturally occurring amino acid
  VARIANT 466: note = X can be any naturally occurring amino acid
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLEWRNTSGLYVLTNDCSNSSIVYEADDVILHTPGCVPCVQDGNTSTCWTPVTPTVAVRYVGATTASIRSHVDLLVGAATMCSALYVGDMCGAVFLVGQAFTFRPRRHQTVQTCNCSLYPGHLSGHRMAWDMMMNWSPAVGMVVAHVLRLPQTLFDIIAGAHWGILAGLAYYSMQGNWAKVAIIMVMFSGVDATTYTTGGSAARGARGLTSLFSVGAKQKLQLVNTNGSWHINSTALNCNXSINTGFIAGLFYYHKFNSTGCPQRLSSCKPITFFXQGWGPLTDANITGPSDDKPYCWHYAPRPCDVVPASSVCGPVYCFTPSPVVVGTTDAKGVPTYTWGENETDVFLLESLRPPSGRWFGCTWMNSTGFVKTCGAPPCNIYGGGGNPHNESDLFCPTDCFRKHPEATYSRCGAGPWLTPRCMVDYPYRLWHYPCTVNFTLFKVRMFVGGFEHRFTAACNWTRGERCDIEDRDRSEQHPLLHSTTELAILPCSFTPMPALSTGLIHLHQNIVDVQYLYGVGSGMVGWALKWEFVILVFLLLADARVCVALWLMLMISQAEA

SEQ ID NO: 89 - protein, 752 aa, Hepatitis C virus
MSTLPKPQRKTKRNTIRRPQDVKFPGGGQIVGGVYVLPRRGPRLGVRATRKTSERSQPRERRQPIPKARRSDGRSWAQPGYPWPLYGNEGCGWAGWLLSPRGSRPSWGPNDPRRRSRNLGKVIDTLTCGFADLMGYIPIVGAPVGGVARALAHGVRALEDGINFATGNLPGCSFSIFLLALLSCLIHPAASLEWRNTSGLYILTNDCPNSSIVYEADDVILHTPGCIPCVQDGNTSTCWTSVSPTVAVRYVGATTASIRSHVDLLVGAATLCSALYVGDMCGAVFLVGQAFTFRPRRHQTVQTCNCSLYPGHLTGHRMAWDMMMNWSPAVGMVVAHVLRMPQTVFDIIAGAHWGILAGLAYYSMQGNWAKVAIIMVMFSGVDAETHTTGGTAARNAFTLTGLFTQGARQKLELINTNGSWHINRTALNCNESLNTGFIAGLFYLHKFNSTGCPERLSSCKPITFFRQGWGSLTDANITGPSDDKPYCWHYAPRPCEVVPALNVCGPVYCFTPSPVVVGTTDRQGVPTYTWGENETDVFLLRSLRPPSGQWFGCTWMNSTGFVKTCGAPPCDIYGGGGNRCNESDLFCPTDCFRKHPEATYSRCGAGPWLTPRCLVDYPYRLWHYPCTVNFTLFKVRMFVGGFEHRFTAACNWTRGERCNIEDRDRSEQHPLLHSTTELAILPCSFTPMPALSTGLIHLHQNIVDVQYLYGVGSGVVGWALRWEFVVLVFLLLADARVCVALWLMLMISQAEA

SEQ ID NO: 90 - protein, 752 aa, Hepatitis C virus
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLVHPAASLEWRNTSGLYVLTNDCSNSSIVYEADDVILHTPGCVPCVQDDNTSTCWTPVTPTVAVRYVGATTASIRSHVDLLVGAATLCSALYVGDMCGAVFLVGQAFTFRPRRHQTVQTCNCSLYPGHVSGHRMAWDMMMNWSPAVGMVVAHILRLPQTLFDILAGAHWGILAGLAYYSMQGNWAKVAIVMIMFSGVDAETYVTGGSVAHSARGLTSLFSMGAKQKLQLVNTNGSWHINSTALNCNESINTGFIAGLFYYHKFNSTGCPQRLSSCKPIISFRQGWGPLTDANITGPSDDRPYCWHYAPRPCSVVPASSVCGPVYCFTPSPVVVGTTDIKGKPTYNWGENETDVFLLESLRPPSGRWFGCAWMNSTGFLKTCGAPPCNIYGGEGDPENETDLFCPTDCFRKHPEATYSRCGAGPWLTPRCMVDYPYRLWHYPCTVNFTLFKVRMFVGGFEHRFTAACNWTRGERCNIEDRDRSEQHPLLHSTTELAILPCSFTPMPALSTGLIHLHQNIVDVQYLYGVGSDMVGWALKWEFVILVFLLLADARVCVALWLMLMVSQAEA

SEQ ID NO: 91 - protein, 752 aa, Hepatitis C virus
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLEWRNTSGLYVLTNDCSNSSIVYEADDVILHTPGCVPCVQDGNTSTCWTPVTPTVAVRYVGATTASIRSHVDLLVGAATMCSALYVGDMCGAVFLVGQAFTFRPRRHQTVQTCNCSLYPGHLSGHRMAWDMMMNWSPAVGMVVAHVLRLPQTLFDIMAGAHWGILAGLAYYSMQGNWAKVAIIMVMFSGVDAHTYTTGGTASRHTQAFAGLFDIGPQQKLQLVNTNGSWHINSTALNCNESINTGFIAGLFYYHKFNSTGCPQRLSSCKPITFFRQGWGPLTDANITGPSDDRPYCWHYAPRPCDIVPASSVCGPVYCFTPSPVVVGTTDARGVPTYTWGENEKDVFLLKSQRPPSGRWFGCSWMNSTGFLKTCGAPPCNIYGGEGNPHNESDLFCPTDCFRKHPETTYSRCGAGPWLTPRCMVDYPYRLWHYPCTVDFRLFKVRMFVGGFEHRFTAACNWTRGERCDIEDRDRSEQHPLLHSTTELAILPCSFTPMPALSTGLIHLHQNIVDVQYLYGVGSGMVGWALKWEFVILVFLLLADARVCVALWLMLMISQTEA

SEQ ID NO: 92 - protein, 752 aa, Hepatitis C virus
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGQNDPRRRSRNLGKVIDTLTCGFADLMGYIPLVGAPVGGVARALAHGVRALEDGINFATGNLPGCSFSIFLLALFSCLIHPAASLEWRNTSGLYVLTNDCSNSSIVYEADDVILHTPGCVPCVQDGNTSTCWTPVTPTVAVRYVGATTASIRSHVDLLVGAATMCSALYVGDMCGAVFLVGQAFTFRPRRHQTVQTCNCSLYPGHLSGHRMAWDMMMNWSPAVGMVVSHVLRLPQTLFDIIAGAHWGILAGLAYYSMQGNWAKVAVIMVMFSGVDAETYITGGSAAHGVSTLTSLFSSGPQQKLQLVKTNGSWHINSTALNCNESINTGFIAGLFYYHKFNSTGCPQRLSSCKPITFFRQGWGSLTDANVTGASADKPYCWHYAPRPCDVVPALNVCGPVYCFTPSPVVVGTTDRKGVPTYNWGENESDVFLLESLRPPSGRWFGCAWMNSTGFLKTCGAPPCNIYGGGGNPNNESHLFCPTDCFRKHPDATYSRCGAGPWLTPRCMVDYPYRLWHYPCTVNFTLFKVRMFVGGFEHRFTAACNWTRGERCNIEDRDRSEQHPLLHSTTELAILPCSFTPMPALSTGLIHLHQNIVDVQYLYGVGSGMVGWALKWEFVILIFLLLADARVCVALWLMLMISQAEA

SEQ ID NO: 93 - protein, 752 aa, Hepatitis C virus
MSTLPKPQRKTKRNTIRRPQDVKFPGGGQIVGGVYVLPRRGPRLGVCATRKTSERSQPRRRRQPIPKARQSGGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLEWRNTSGLYVLTNDCSNSSIVYEADDVILHTPGCVPCVQNDNISTCWTPVTPTVAVRYVGATTASIRSHVDLLVGAATMCSALYVGDMCGAVFLVGQAFTFRPRRHQTVQTCNCSLYPGHLSGHRMAWDMMMNWFPALGMAVAHVLRVPQTLFDIIAGAHWGILAGLAYYSMQGNWAKVAIIMVMFSGVDAVTYTTGGSAAHATRGLTSLFSVGAQQKLQLVNTNGSWHINSTALNCNESINTGFIAGLFYYHRFNSTGCPQRLSSCKPITFFKQGWGPLTDANISGPSDDKPYCWHYAPRPCKVVPASGVCGPVYCFTPSPVVVGTTDAKGVPTYTWGANDTDVFLLESLRPPGGRWFGCTWMNSTGFVKTCGASPCDIYGGGGNSGNESDLFCPTDCFRKHPEATYSRCGAGPWLTPRCMVDYPYRLWHYPCTVNFTLFKVRMFVGGFEHRFTAACNWTRGERCDIEDRDRSEQHPLLHSTTELAILPCSFTPMPALSTGLIHLHQNIVDVQYLYGVGSGMVGWALKWEFVILIFLLLADRRVCVALWLMLMITQAEA

SEQ ID NO: 94 - protein, 752 aa, Hepatitis C virus
MSTLPKPQRKTKRNTVCRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLEWRNVSGLYILTNDCPNSSIVYEADDVILHTPGCIPCVQDGNTSTCWTALTPTVAVRYVGATTASIRSHVDLLVGAATMCSALYVGDMCGAVFLVGQAFTFRPRRHQTVQTCNCSLYPGHLTGHRMAWDMMMNWSPAVGMVVAHVLRLPQTLFDIIAGAHWGILAGLAYYSMQGNWAKVIIIMVMFSGVDATTHVTGGTAGLTAFRLTGLFTVGPQQKLQLVNTNGSWHINRTALNCNDSLNTGFIAGLFRFHKFNSTGCPEMLSSCKPITSFKQGWGPLTDANITIPSDDRPYCWHYPPRSCEVVPALSVCGPVYCFTPSPVVVGTTDAKGVPTYTWGENETDVFLLKSLRPPGGRWFGCTWMNSTGFVQTCGAPPCNIYGGGGDLKNESDLFCPTDCFRKHPEATYSRCGAGPWLTPRCMVDYPYRLWHYPCTVNFTLFKVRMFVGGFEHRFTAACNWTRGERCDIEDRDRSELHPLLHSTTELAILPCSFTPMPALSTGLIHLHQNIVDVQYLYGVGSGMVGWAVKWEFVILVFLLLADARVCVALWLMLMISQAEA

SEQ ID NO: 95 - protein, 752 aa, Hepatitis C virus
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLQWRNTSGLYVLTNDCSNSSIVYEADDVILHTPGCVPCVQDGNTSTCWTPVTPTVAVRYVGATTASIRSHVDLLVGAATMCSALYVGDMCGAVFLVGQAFTFRPRRHQTVQTCNCSLYPGHLSGHRMAWDMMMNWSPAAGMVVAHILRLPQTLFDIIAGAHWGILAGLAYYSMQGNWAKVAIIMVMFSGVDATTYTSGGSVAQQARGLADLFSVGAKQNLQLVNTNGSWHINSTALNCDDSINTGFIAGLFYYHKFNSTGCPQRLSDCKPITFFKQGWGPLTDANITGPSDDKPYCWHYAPRRCGVVPASSVCGPVYCFTPSPVVVGTTDAKGVPTYTWGANETDVFLLESLRPPSGRWFGCAWMNSTGFLKTCGAPPCNIYGGGGNPHNESDLFCPTDCFRKHPEATYSRCGAGPWLTPRCMVDYPYRLWHYPCTVNFTLFKMRTFVGGFEHRFTAACNWTRGERCDIEDRDRSEQHPLLHSTTELAILPCSFTPMPALSTGLIHLHQNIVDVQYLYGVGSGMVGWALKWEFVILVFLLLADARVCVALWLMLMISQAEA

SEQ ID NO: 96 - protein, 752 aa, Hepatitis C virus; identical to SEQ ID NO: 95

SEQ ID NO: 97 - protein, 752 aa, Hepatitis C virus
MSTLPKPQRKTKRNTIRRPQDVKFPGGGQIVGGVYVLPRRGPRLGVCATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLVHPAASLEWRNTSGLYVLTNDCPNSSIVYEADDVILHTPGCIPCVQDGNKSTCWTSVTPTVAVKYVGATTASIRSHVDLLVGAATMCSALYVGDMCGAVFLVGQAFTFRPRRHQTVQTCNCSLYPGHLSGHRMAWDMMMNWSPAVGMVVAHVLRLPQTLFDIIAGAHWGILAGLAYYSMQGNWAKVAIIMVMFSGVDATTYTTGGNAARGASGIVSLFTPGAKQNLQLVNTNGSWHINRTALNCNDSINTGFIAGLIYYHKFNSTGCPQRLSSCKPITFFRQGWGSLTDANITGPSDDKPYCWHYPPRPCDTIRASSVCGPVYCFTPSPVVVGTTDAKGAPTYNWGANETDMFLLQSLRPPSGRWFGCTWMNSTGFTKTCGAPPCNIYGGGGNLNNESDLFCPTDCFRKHPEATYSRCGAGPWLTPRCLVDYPYRLWHYPCTVNFTLFRMRTFVGGFEHRFTAACNWTRGERCNIEDRDRSEQHPLLHSTTELAILPCSFTPMPALSTGLIHLHQNIVDVQYLYGIGSGVVGWALKWEFVILVFLLLADARVCVALWLMLMISQAEA

SEQ ID NO: 98 - protein, 752 aa, Hepatitis C virus
MSTLPKPQRKTKRNTIRRPQDVKFPGGGVIYVGVYVLPRRGPRLGVRATRKTSERSQPRGRRKPIPKARRSEGRSWAQPGYPWPLYGNEGCGWAGWLLSPRGSRPNWAPNDPRRRSRNLGKVIDTLTCGFADLMGYIPLVGAPLGGAARALAHGVRALEDGINFATGNLPGCSFSIFLLALFSCLIHPAASLEWRNTSGLYVLTNDCSNSSIVYEADDVILHTPGCIPCVQDGNTSTCWTPVTPTVAVRYVGATTASIRSHVDLLVGAGTMCSALYVGDMCGPVFLVGQAFTFRPRRHRTVQTCNCSLYPGHLSGQRMAWDMMMNWSPAVGMVVAHILRLPQTLFDVVAGAHWGIIAGLAYYSMQGNWAKVAIIMVMFSGVDASTHVTAGQAARNAYGITSLFSVGAKQNLQLINTNGSWHINRTALNCNESINTGFIAGLFYYHKFNSTGCPQRLSSCKPITFFKQGWGPLTDANITGPSDDKPYCWHYAPRPCGIVPALNVCGPVYCFTPSPVVVGTTDAKGAPTYTWGANKTDVFLLESLRPPSGRWFGCTWMNSTGFVKTCGAPPCNIYGDGRDAQNESDLFCPTDCFRKHPEATYSRCGAGPWLTPRCLVDYPYRLWHYPCTVNFTLFKVRMFVGGFEHRFTAACNWTRGERCDIEDRDRSEQHPLLHSTTELAILPCSFTPMPALSTGLIHLHQNIVDVQYLYGIGSGMVGWALKWEFVILIFLLLADARVCVALWLILTISQAEA

SEQ ID NO: 99 - protein, 754 aa, Hepatitis C virus
MSTLPKPKRQTKRNTLRRPKNVKFPAGGQIVGEVYVLPRRGPQLGVREVRKTSERSQPRGRRQPTPKARPREGRSWAQPGYPWPLYGNEGCGWAGWLLPPRGSRPSWGQNDPRRRSRNLGKVIDTLTCGFADLMGYIPLIGAPVGGVARALAHGVRALEDGVNYATGNLPGCSFSIFLLALFSCLTCPASSLEYRNASGLYLLTNDCSNRSIVYEADDVILHLPGCVPCVETDNNNTSCWTPISPTVAVKHPGVTTASIRNHVNMLVAPPTLCSALYVEDAFGAVSLVGQAFTFRPRQHKTVQTCNCSIYPGHVSGHRMAWDMMMNWSPAIGLVISHLMRLPQTFFDLVVGAHWGVMAGLAYFSMQGNWAKVVIVLIMFSGVDATTHTTGGSAAQATAGFTSFFTRGPSQNLQLVNSNGSWHINSTALNCNDSLNTGFIAGLFYYHKFNSSGCPERMSSCKPITYFNQGWGPLTDANINGPSEDRPYCWHYPPRPCNITKPLNVCGPVYCFTPSPVVVGTTDIKGLPTYRFGVNESDVFLLTSLRPPQGRWFGCVWMNSTGFVKTCGAPPCNIYGGMKDIEANQTHLKCPTDCFRKHHDATFTRCGSGPWLTPRCLVDYPYRLWHYPCTVNFSIFKVRMFVGGHEHRFSAACNWTRGERCDLEDRDRSEQQPLLHSTTDSLILPCSFTPMRRLSTGLIHLHQNIVDVQYLYGVGSAVVGWALKWEFVVLVFLLLADARVCVALWMMLLISQAEA

SEQ ID NO: 100 - protein, 751 aa, Hepatitis C virus
MSTLPKPQRITKRNINRRPQDVKFPGGGQIVGGVYVLPRRGPKLGVRAVRKTSERSQPRSRRQPIPRARRTEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALLSCLLTPTAGLEYRNASGLYTVTNDCSNGSIVYEAGDVILHLPGCIPCVRLNNASKCWTPVSPTVAVSRPGAATASLRTHVDMMVGAATLCSALYVGDLCGALFLVGQGFSWRHRQHWTVQDCNCSIYPGHLTGHRMAWDMMMNWSPAMTLIVSQVLRLPQTMFDLVIGAHWGVMAGVAYYSMQGNWAKVFLVLCLFSGVDASTTITGGVAASGAFTITSLFSTGAKQPLHLVNTNGSWHINRTALNCNDSLNTGFIAGLLYYHKFNSSGCVERMSACSPLDRFAQGWGPLGPANISGPSSEKPYCWHYAPRPCDTVPAQSVCGPVYCFTPSPVVVGATDKRGAPTYTWGENESDVFLLESARPPTEPWFGCTWMNGSGYVKTCGAPPCHIYGGREGKSNNSLVCPTDCFRKHPDATYNRCGAGPWLTPRCLVDYPYRLWHYPCTVNYTIFKVRMFVGGLEHRFNAACNWTRGERCNLEDRDRSEMYPLLHSTTEQAILPCSFVPIPALSTGLIHLHQNIVDVQYLYGISSGLVGWAIKWEFVILIFLLLADARVCVVLWMMMLISQAEA

SEQ ID NO: 101 - protein, 750 aa, Hepatitis C virus
MSTNPKPQRLTKRNTVRRPQNVKFPGGGQIVGGVYLLPRRGPRLGVRGTRKSSERSQPRGRRQRIPKAASSQGKAWGKPGYPWPLYGNEGCGWAGWLLSPRGSRPTWGPTDPRHRSRNLGKVIDTMTCGFADLMGYIPVLGAPLGGVARALAHGVRVLEDGVNYATGNLPGCSFSIFLLALLSCLTVPASAYEVRNSSGVYHLTNDCPNASIVYETDNAILHEPGCVPCVREGNTSRCWEPVAPTLAVRYRGALTDDLRTHIDLVVASATLCSALYVGDICGAIFIASQAVLWKPGGGRIVQDCNCSIYPGHVTGHRMAWDMMQNWAPALSMVAAYAVRVPGVIITTVAGGHWGVLFGLAYFGMAGNWAKVILIMLLMSGVDAETMAVGARAAHTTGALVSLLNPGPSQRLQLINTNGSWHINRTALNCNDSLQTGFIAALFYTHRFNSSGCPERMASCKPLSDFDQGWGPLWYNSTERPSDQRPYCWHYAPSPCGIVPAKDVCGPVYCFTPSPVVVGTTDRRGVPTYTWGENESDVFLLNSTRPPQGSWFGCSWMNTTGFTKTCGGPPCKIRPQGAQSNTSLTCPTDCFRKHPRATYSACGSGPWLTPRCMVHYPYRLWHYPCTVNFTIHKVRLYIGGVEHRLDAACNWTRGERCDLEDRDRVDMSPLLHSTTELAILPCSFVPLPALSTGLIHLHQNIVDAQYLYGLSPAIISWAIRWEWVVLVFLLLADARICACLWMMMLMAQAEA

SEQ ID NO: 102 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 325 aa, Homo sapiens
STKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 104 - protein, 246 aa, Homo sapiens
HKPSNTKVDKRVELKTPLGDTTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 383 aa, Homo sapiens
PTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 106 - protein, 270 aa, Homo sapiens
VTSTLTIKSDWLGESMFTCRVDHRGLTFQQNASSMCVPDQDTAIRVFAIPPSFASIFLTKSTKLTCLVTDLTTYSVTISWTREENGAVKTHTNISESHPNATFSAVGEASICEDDWSGERFTCTVTHTDLPSPLKQTISRPKGVALHRPVYLLPPARLNLRESATITCLVTGFSPADVFVEWMQRGEPLSPQKYVTSAPMPEPQAPGRYFAHSILTVSEEEWNTGGTYTCVVAHEALPNRVTERTVDKSTGKPTLYNVSLVMSDTAGTCY

SEQ ID NO: 107 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 108 - protein, 222 aa, Homo sapiens
ADPCDSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 109 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 110 - protein, 14 aa, synthetic construct
  VARIANT 4..5: note = Human IgG1 Fc may be between residues 4 and 5.
DAETLEVLFQGPET

SEQ ID NO: 111 - protein, 14 aa, synthetic construct
  VARIANT 4..5: note = Human IgG1 Fc may be between residues 4 and 5.
DAQTLEVLFQGPQT

SEQ ID NO: 112 - protein, 14 aa, synthetic construct
  VARIANT 4..5: note = Human IgG1 Fc may be between residues 4 and 5.
DAGTLEVLFQGPGT

SEQ ID NO: 113 - protein, 14 aa, synthetic construct
  VARIANT 4..5: note = Human IgG1 Fc may be between residues 4 and 5.
DAETLEVLFQGPET

SEQ ID NO: 114 - protein, 14 aa, synthetic construct
  VARIANT 4..5: note = Human IgG1 Fc may be between residues 4 and 5.
DAETLEVLFQGPET

SEQ ID NO: 115 - protein, 14 aa, synthetic construct
  VARIANT 4..5: note = Human IgG1 Fc may be between residues 4 and 5.
DAETLEVLFQGPET

SEQ ID NO: 116 - protein, 10 aa, synthetic construct
  VARIANT 2..3: note = Human IgG1 Fc may be between residues 2 and 3.
ETLEVLFQGP

SEQ ID NO: 117 - protein, 815 aa, synthetic construct
MDAMKRGLCCVLLLCGAVFVSPSYQVRNSTGLYHVTNDCPNSSIVYEAADAILHTPGCVPCVREGNTSRCWVAMTPTVATRDGKLPTTQLRRHIDLLVGSATLCSALYVGDLCGSIFLVGQMFTFSPRRHWTTQDCNCSLYPGHITGHRMAWDMMMNWSPTAALITAQLLRIPQAILDMIAGAHWGVLAGIAYFSMVGNWAKVLVVLLLFAGVDAQTDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLEVLFQGPQTHVTGGRAAHITAGLTSLFSPGPSQKLQLVNTNGSWHINSTALNCNDSLKTGWIAGLLYSYKFNSSGCPERLASCRRLTDFAQGWGPISHANGSGPDERPYCWHYPPRPCGIVPAKSVCGPVYCFTPSPVVVGTTDKSGAPTYNWGENDWDVFVLNNTRPPLGNWFGCTWMNSTGFTKVCGAPPCVIGGAGNNTLRCPTDCFRKHPDATYSRCGSGPWITPRCLVDYPYRLWHYPCTVNYSIFKIRMYLGGVEHRLEAACNWTRGERCDLEDRDRSELSPLLLSTTQWQVLPCSFTTLPALSTGLIHLHQNIVDVQYLYGVGSSVASWAIKWDYVVLLFLLLADARICSCLWMMLLISQAEA

SEQ ID NO: 118 - protein, 815 aa, synthetic construct
MDAMKRGLCCVLLLCGAVFVSPSYQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDAETDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLEVLFQGPETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEA

SEQ ID NO: 119 - protein, 821 aa, synthetic construct
MDAMKRGLCCVLLLCGAVFVSPSLEWRNTSGLYVLTNDCSNSSIVYEADDVILHTPGCVPCVQDDNTSTCWTPVTPTVAVRYVGATTASIRSHVDLLVGAATLCSALYVGDMCGAVFLVGQAFTFRPRRHQTVQTCNCSLYPGHVSGHRMAWDMMMNWSPAVGMVVAHILRLPQTLFDILAGAHWGILAGLAYYSMQGNWAKVAIVMIMFSGVDAETDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLEVLFQGPETYVTGGSVAHSARGLTSLFSMGAKQKLQLVNTNGSWHINSTALNCNESINTGFIAGLFYYHKFNSTGCPQRLSSCKPIISFRQGWGPLTDANITGPSDDRPYCWHYAPRPCSVVPASSVCGPVYCFTPSPVVVGTTDIKGKPTYNWGENETDVFLLESLRPPSGRWFGCAWMNSTGFLKTCGAPPCNIYGGEGDPENETDLFCPTDCFRKHPEATYSRCGAGPWLTPRCMVDYPYRLWHYPCTVNFTLFKVRMFVGGFEHRFTAACNWTRGERCNIEDRDRSEQHPLLHSTTELAILPCSFTPMPALSTGLIHLHQNIVDVQYLYGVGSDMVGWALKWEFVILVFLLLADARVCVALWLMLMVSQAEA

SEQ ID NO: 120 - protein, 821 aa, synthetic construct
MDAMKRGLCCVLLLCGAVFVSPSLEWRNTSGLYILTNDCPNSSIVYEADDVILHTPGCIPCVQDGNTSTCWTSVSPTVAVRYVGATTASIRSHVDLLVGAATLCSALYVGDMCGAVFLVGQAFTFRPRRHQTVQTCNCSLYPGHLTGHRMAWDMMMNWSPAVGMVVAHVLRMPQTVFDIIAGAHWGILAGLAYYSMQGNWAKVAIIMVMFSGVDAETDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLEVLFQGPETHTTGGTAARNAFTLTGLFTQGARQKLELINTNGSWHINRTALNCNESLNTGFIAGLFYLHKFNSTGCPERLSSCKPITFFRQGWGSLTDANITGPSDDKPYCWHYAPRPCEVVPALNVCGPVYCFTPSPVVVGTTDRQGVPTYTWGENETDVFLLRSLRPPSGQWFGCTWMNSTGFVKTCGAPPCDIYGGGGNRCNESDLFCPTDCFRKHPEATYSRCGAGPWLTPRCLVDYPYRLWHYPCTVNFTLFKVRMFVGGFEHRFTAACNWTRGERCNIEDRDRSEQHPLLHSTTELAILPCSFTPMPALSTGLIHLHQNIVDVQYLYGVGSGVVGWALRWEFVVLVFLLLADARVCVALWLMLMISQAEA

SEQ ID NO: 121 - protein, 227 aa, Staphylococcus aureus
MKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEI

SEQ ID NO: 122 - protein, 448 aa, Streptococcus dysgalactiae
MEKEKKVKYFLRKSAFGLASVSAAFLVGSTVFAVDSPIEDTPIIRNGGELTNLLGNSETTLALRNEESATADLTAAAVADTVAAAAAENAGAAAWEAAAAADALAKAKADALKEFNKYGVSDYYKNLINNAKTVEGVKDLQAQVVESAKKARISEATDGLSDFLKSQTPAEDTVKSIELAEAKVLANRELDKYGVSDYYKNLINNAKTVEGVKALIDEILAALPKTDTYKLILNGKTLKGQTTTEAVDAATAEKVFKQYANDNGVDGEWTYDDATKTFTVTEKPEVIDAPELTPAVTTYKLVINGKTLKGETTTKAVDAETAEKAFKQYANDNGVDGVWTYDDATKTFTVTEMVTEVPGDAPTEPEKPEASIPLVPLTPATPIAKDDAKKDDTKKEDAKKPEAKKEDAKKAETLPTTGEGSNPFFTAAALAVMAGAGALAVASKRKED

SEQ ID NO: 123 - protein, 992 aa, Finegoldia magna
MKINKKLLMAALAGAIVVGGGANAYAAEEDNTDNNLSMDEISDAYFDYHGDVSDSVDPVEEEIDEALAKALAEAKETAKKHIDSLNHLSETAKKLAKNDIDSATTINAINDIVARADVMERKTAEKEEAEKLAAAKETAKKHIDELKHLADKTKELAKRDIDSATTINAINDIVARADVMERKTAEKEEAEKLAAAKETAKKHIDELKHLADKTKELAKRDIDSATTIDAINDIVARADVMERKLSEKETPEPEEEVTIKANLIFADGSTQNAEFKGTFAKAVSDAYAYADALKKDNGEYTVDVADKGLTLNIKFAGKKEKPEEPKEEVTIKVNLIFADGKTQTAEFKGTFEEATAKAYAYADLLAKENGEYTADLEDGGNTINIKFAGKETPETPEEPKEEVTIKVNLIFADGKIQTAEFKGTFEEATAKAYAYANLLAKENGEYTADLEDGGNTINIKFAGKETPETPEEPKEEVTIKVNLIFADGKTQTAEFKGTFEEATAEAYRYADLLAKVNGEYTADLEDGGYTINIKFAGKEQPGENPGITIDEWLLKNAKEEAIKELKEAGITSDLYFSLINKAKTVEGVEALKNEILKAHAGEETPELKDGYATYEEAEAAAKEALKNDDVNNAYEIVQGADGRYYYVLKIEVADEEEPGEDTPEVQEGYATYEEAEAAAKEALKEDKVNNAYEVVQGADGRYYYVLKIEDKEDEQPGEEPGENPGITIDEWLLKNAKEDAIKELKEAGISSDIYFDAINKAKTVEGVEALKNEILKAHAEKPGENPGITIDEWLLKNAKEAAIKELKEAGITAEYLFNLINKAKTVEGVESLKNEILKAHAEKPGENPGITIDEWLLKNAKEDAIKELKEAGITSDIYFDAINKAKTIEGVEALKNEILKAHKKDEEPGKKPGEDKKPEDKKPGEDKKPEDKKPGEDKKPEDKKPGKTDKDSPNKKKKAKLPKAGSEAEILTLAAAALSTAAGAYVSLKKRK